(19) United States
(12) Patent Application Publication
(10) Pub. No.: US 2021/0260057 A1
(43) Pub. Date: Aug. 26, 2021

(54) COMPOSITIONS AND METHODS TARGETING GLUTAMINE AND ITS METABOLISM FOR DIAGNOSING AND TREATING CANCER AND THERAPY-ASSOCIATED SIDE EFFECTS

(71) Applicant: CEDARS-SINAI MEDICAL CENTER, Los Angeles, CA (US)

(72) Inventors: Neil Bhowmick, Beverly Hills, CA (US); Rajeev Mishra, Los Angeles, CA (US); Bethany Smith, Lancaster, CA (US)

(73) Assignee: CEDARS-SINAI MEDICAL CENTER, Los Angeles, CA (US)

(21) Appl. No.: 17/257,531

(22) PCT Filed: Jul. 12, 2019
(86) PCT No.: PCT/US19/41603

§ 371 Date: Dec. 31, 2020

Related U.S. Application Data
(60) Provisional application No. 62/697,335, filed on Jul. 12, 2018.

(51) Int. Cl. A61K 31/501; A61K 31/198; A61K 45/06; A61K 31/433; G01N 33/68

(52) U.S. Cl. CPC A61K 31/501; A61K 31/198; G01N 33/6812; A61K 31/433; A61K 45/06

(57) ABSTRACT

The present disclosure provides compositions, markers, and methods for diagnosing and treating cancer and other conditions. The method includes administering at least one androgen deprivation therapy to the subject; and administering a therapeutically effective amount of at least one inhibitor to the subject, wherein the at least one inhibitor is selected from the group including: at least one macropinocytosis inhibitor, at least one glutamine metabolism inhibitor, at least one glutaminase inhibitor, and any combinations thereof.

### CROSS-REFERENCE TO RELATED APPLICATIONS

This application includes a claim of priority under 35 U.S.C. § 119(e) to U.S. provisional patent application No. 62/697,335 filed Jul. 12, 2018, the entirety of which is hereby incorporated by reference.

### STATEMENT REGARDING FEDERALLY SPONSORED RESEARCH OR DEVELOPMENT

This invention was made with government support under Grant Nos. CA108646 and CA098912 awarded by National Institutes of Health and Grant No. BX001040 awarded by the Department of Veterans Affairs. The government has certain rights in the invention.

### FIELD OF THE INVENTION

The present disclosure relates to compositions, markers, and methods for diagnosing and treating cancer and other conditions.

### BACKGROUND

Many people have or are at risk of developing cancer and other associated diseases. There is a need for methods for identification/discover of markers for cancer and other diseases, and for diagnosing and/or prognosing and/or predicting progression of and/or treating cancer and other diseases.

### SUMMARY OF THE INVENTION

The following embodiments and aspects thereof are described and illustrated in conjunction with compositions and methods which are meant to be exemplary and illustrative, not limiting in scope.

Various embodiments of the present invention provide for a method of treating, reducing the severity of, slowing the progression of, or inhibiting the progression of a cancer, cancer metastasis, tumor or tumor metastasis, or sensitizing a cancer, cancer metastasis, tumor or tumor metastasis to a cancer therapy, in a subject in need thereof, comprising:

administering a glutaminase inhibitor, a glutamine metabolism inhibitor, a micropinocytosis inhibitor or a combination thereof to the subject to treat, reduce the severity of, slow the progression of, or inhibit the progression of the cancer, cancer metastasis, tumor or tumor metastasis, or to sensitize the cancer, cancer metastasis, tumor or tumor metastasis to the cancer therapy.

In various embodiments, the glutaminase inhibitor can be selected from the group consisting of: bis-2(5-phenylacetamido-1,3,4-thiadiazol-2-yl)ethyl sulfide (BPTES), and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839), salts thereof, and combinations thereof.

In various embodiments, the glutamine metabolism inhibitor can be L-gamma-glutamyl-p-nitroanilide (GPNA) or salts thereof.

In various embodiments, the method can be treating, reducing the severity of, slowing the progression of, or inhibiting the progression of prostate cancer, and the method can further comprise administering an androgen deprivation therapy to the subject to treat, reduce the severity of, slow the progression of, or inhibit the progression of the prostate cancer.

In various embodiments, the androgen deprivation therapy can comprise surgical castration or an antiandrogen. In various embodiments, the antiandrogen can be an androgen receptor (AR) antagonist, androgen synthesis inhibitor, antigonadotropin, or combinations thereof. In various embodiments, the androgen deprivation therapy can comprise an androgen receptor signaling inhibitor, androgen receptor inhibitor, or both. In various embodiments, the prostate cancer can be androgen deprivation therapy resistant prostate cancer.

In various embodiments, the method can be treating, reducing the severity of, slowing the progression of, or inhibiting the progression of cancer, and the method can further comprise administering a cancer therapy to the subject to treat, reduce the severity of, slow the progression of, or inhibit the progression of the cancer.

In various embodiments, the method can further comprise administering a CD105 inhibitor to the subject.

In various embodiments, the method can further comprise administering an immune checkpoint inhibitor to the subject. In various embodiments, the immune checkpoint inhibitor can be anti-PD1 inhibitor, anti-PDL1 inhibitor, or anti-CD47 inhibitor. In various embodiments, the anti-PD1 inhibitor can be selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, PF-06801591, anti-PD1 antibody expressing pluripotent killer T lymphocytes (PIK-PD-1), autologous anti-EGFRvIII 4SCAR-IgT cells, and combinations thereof. In various embodiments, the anti-PDL1 inhibitor can be selected from the group consisting of BGB-A333, CK-301, FAZ053, KN035, MDX-1105, MSB2311, SHR-1316, atezolizumab, avelumab, durvalumab, BMS-936559, CK-301, M7824, and combinations thereof. In various embodiments, the anti-CD47 inhibitor can be selected from the group consisting of anti-CD47 monoclonal antibody AO-176, anti-CD47 monoclonal antibody CC-90002, anti-CD47 monoclonal antibody Hu5F9-G4, anti-CD47 monoclonal antibody IBI188, anti-CD47 monoclonal antibody SHR-1603, anti-CD47 monoclonal antibody SRF231, CD47 antagonist ALX-148, chimeric humanized anti-CD47 antibody, and combinations thereof.

In various embodiments, the method can be treating, reducing the severity of, slowing the progression of, or inhibiting the progression of cancer, and the method can further comprises administering a CD105 inhibitor to the subject.

In various embodiments, the method can further comprise administering an immune checkpoint inhibitor to the subject. In various embodiments, the immune checkpoint inhibitor can be anti-PD1 inhibitor, anti-PDL1 inhibitor, or anti-CD47 inhibitor. In various embodiments, the anti-PD1 inhibitor can be selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, PF-06801591, anti-PD1 antibody expressing pluripotent killer T lymphocytes (PIK-PD-1), autologous anti-EGFRvIII 4SCAR-IgT cells, and combinations thereof. In various embodiments, the anti-PDL1 inhibitor can be selected from the group consisting of BGB-A333, CK-301, FAZ053, KN035, MDX-1105, MSB2311, SHR-1316, atezolizumab, avelumab, durvalumab, BMS-936559, CK-301, M7824, and combinations thereof. In various embodiments, the anti-CD47 inhibitor can be selected from the group consisting of anti-CD47 monoclonal antibody AO-176, anti-CD47 monoclonal antibody CC-90002, anti-CD47 monoclonal antibody Hu5F9-G4, anti-CD47 monoclonal antibody IBI188, anti-CD47 monoclonal antibody SHR-1603, anti-CD47 monoclonal antibody SRF231, CD47 antagonist ALX-148, chimeric humanized anti-CD47 antibody, and combinations thereof.

In various embodiments, the method can be sensitizing a cancer to a cancer therapy, and the cancer is prostate cancer, and the cancer therapy is androgen deprivation therapy and the method sensitizes the prostate cancer to the androgen deprivation therapy.

In various embodiments, the method can be sensitizing a cancer to a cancer therapy, and the cancer therapy is androgen deprivation therapy, radiation therapy, chemotherapy or combinations thereof.

In various embodiments, the method can be treating, reducing the severity of, slowing the progression of, or inhibiting the progression of cancer metastasis, and the method can further comprise administering a CD105 inhibitor; and administering a cancer therapy.

In various embodiments, the cancer can be selected from the group consisting of prostate cancer, pancreatic cancer, breast cancer, lung cancer, colon cancer, liver cancer, and combinations thereof. In various embodiments, the cancer can be a Ras mutated cancer. In various embodiments, the cancer can be an adenocarcinoma or a neuroendocrine cancer.

Various embodiments of the present invention provide for a method of treating, reducing the severity of, slowing the progression of, or inhibiting the progression of a disease or condition, or preventing or reducing activation of stellate cells, in a subject in need thereof, comprising: administering a glutaminase inhibitor, a glutamine metabolism inhibitor, a micropinocytosis inhibitor or a combination thereof to the subject to treat, reduce the severity of, slow the progression of, or inhibit the progression of the condition, wherein the condition is selected from the group consisting of liver fibrosis, pancreatic fibrosis, cancer associated depression and combinations thereof.

In various embodiments, the glutaminase inhibitor can be selected from the group consisting of: bis-2(5-phenylacetamido-1,3,4-thiadiazol-2-yl)ethyl sulfide (BPTES), and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839), salts thereof, and combinations thereof.

In various embodiments, the glutamine metabolism inhibitor can be L-gamma-glutamyl-p-nitroanilide (GPNA) or salts thereof.

In various embodiments, the method can be for preventing or reducing activation of stellate cells and the stellate cells are selected from the group consisting of hepatic stellate cells, pancreatic stellate cells, and combinations thereof.

Various embodiments of the present invention provide for a method for determining whether the amount glutamine, a glutamine metabolite or both is elevated in a subject in need thereof, comprising: detecting the glutamine, glutamine metabolite, or both; quantifying the amount of glutamine, glutamine metabolite, or both; comparing the amount of the glutamine to a reference amount of glutamine, or comparing the amount of the glutamine metabolite to a reference amount of glutamine metabolite, or both; and determining whether the amount glutamine, or a glutamine metabolite or both is elevated compared to the reference amount of glutamine, the reference amount glutamine metabolite, or both, in the subject in need thereof.

Various embodiments of the present invention provide for a method for diagnosing a disease or condition in a subject in need thereof, comprising detecting the glutamine, glutamine metabolite, or both; quantifying the amount of glutamine, or glutamine metabolite, or both; comparing the amount of the glutamine to a reference amount of glutamine, or comparing the amount of the glutamine metabolite to a reference amount of glutamine metabolite, or both; and determining that the subject has the disease or condition when the amount glutamine, or glutamine metabolite or both is elevated compared to the reference amount of glutamine, or the reference amount glutamine metabolite, or both, in the subject in need thereof, wherein the disease or condition is cancer, cancer metastasis, tumor, tumor metastasis, liver fibrosis, pancreatic fibrosis, cancer associated depression or combinations thereof.

Various embodiments of the present invention provide for method for determining the risk of developing a condition in a subject in need thereof, comprising: detecting the glutamine, glutamine metabolite, or both; quantifying the amount of glutamine, or glutamine metabolite, or both; comparing the amount of the glutamine to a reference amount of glutamine, or comparing the amount of the glutamine metabolite to a reference amount of glutamine metabolite, or both; and determining that the subject has an increased risk in developing the disease or condition when the amount glutamine, or glutamine metabolite or both is elevated compared to the reference amount of glutamine, the reference amount glutamine metabolite, or both, in the subject in need thereof, wherein the disease or condition is cancer, cancer metastasis, tumor, tumor metastasis, liver fibrosis, pancreatic fibrosis, cancer associated depression or combinations thereof.

In various embodiments, quantifying can be performed using a technique selected from the group consisting of: spectral magnetic resonance imaging, sample assay, and combinations thereof.

In various embodiments, the glutamine metabolite can be selected from the group consisting of: glutamate, glutathione, γ-aminobutyrate, α-ketoglutarate, succinate, fumarate, malate, acetyl-CoA, 2-hydroxyglutarate and combinations thereof.

In various embodiments, the subject has been treated with an androgen deprivation therapy.

In various embodiments, the method can comprise determining that the subject has an indolent or aggressive form of the disease or condition when an elevated amount of glutamine in the subject compared to a reference amount of glutamine is detected.

Various embodiments of the present invention provide for a composition, comprising: an androgen deprivation therapy agent, and an inhibitor selected from the group consisting of a glutaminase inhibitor, a glutamine metabolism inhibitor, a macropinocytosis inhibitor, and combinations thereof.

In various embodiments, the composition can be for use in inhibiting the growth of prostate cancer, delaying the growth of prostate cancer or treating prostate cancer.

In various embodiments, the glutaminase inhibitor can be selected from the group consisting of: bis-2(5-phenylacetamido-1,3,4-thiadiazol-2-yl)ethyl sulfide (BPTES), and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839), salts thereof, and combinations thereof.

In various embodiments, the glutamine metabolism inhibitor can be L-gamma-glutamyl-p-nitroanilide (GPNA) or salts thereof.

Other features and advantages of the invention will become apparent from the following detailed description, taken in conjunction with the accompanying drawings, which illustrate, by way of example, various features of embodiments of the invention.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1F depict epigenetic down-regulation of RASAL3 in prostatic cancer associated fibroblast. (FIG. 1A) Heat map summarizing DNA methylation levels of CpG repeats (blue color indicates hypo-methylation and brown represents hyper-methylation). Top twenty methylated genes significantly differentially expressed between NAF and CAF. Each column represents a fibroblast sample and each row represents the methylation level of indicated gene (n=5). (FIG. 1B) Top: University of California at Santa Cruz Genome Browser screen shot of the indicated region of Chromosome 19 showing the positions of the RASAL3 gene, histone 3 lysine 27 acetylation and human mRNA. Bottom: Locations of predicted CpG sites in Exon 2 by bisulfite sequencing, represented a filled (methylated) or empty circle (unmethylated, n=5). The number of methylated CpGs divided by total number of true CpGs analyzed is given as a percentage on the pie chart. (FIG. 1C) Box plots representing the difference in methylation state of the RASAL3 gene promoter between NAF and CAF as measured by RRBS analysis (n=5). (FIG. 1D) RASAL3 mRNA expression in a panel of primary fibroblast was examined by quantitative rtPCR, normalizing to GAPDH mRNA expression. (FIG. 1E and FIG. 1F) Box plots show relative gene expression level (log 2) of the RASAL3 in normal and prostate cancer patients from indicated data sets. Data represent the mean±SD. *P<0.05 by two-tailed student's t test.

FIGS. 2A-2H show that ras activation stimulates macropinocytosis in prostatic fibroblasts and potentiates growth of adjacent epithelial. (FIG. 2A) Representative western blots of RASAL3, Ras, and Ras effectors in prostatic human NAF/CAF (n≤3). (FIG. 2B) TMR-dextran (red) uptake by CAF display elevated levels of macropinocytosis compared to NAF. DAPI staining (blue) identifies nuclei. (FIG. 2C) CAF that were co-incubated with fluorescent DQ-BSA (green) and TMR-dextran (red) were fixed after 1 hr chase. The fluorescent signal emanating from DQ-BSA with TMR-dextran positive staining (arrowheads) indicate albumin uptake by macropinosomes and subsequent breakdown. (FIG. 2D) Representative western blots of RASAL3, total Ras, Ras-GTP, and phosphorylated-ERK expression by Rasa13-KO and Cas9 control mouse prostatic fibroblasts is shown (n≤3). (FIG. 2E) Representative images show TMR-dextran positive macropinosomes (arrowheads) in Rasa13-KO prostatic mouse fibroblasts compared to Cas9. (FIG. 2F) Representative western blots of mouse prostatic fibroblasts wild type (WT) and expressing Ras^{V12} indicate total Ras, Ras-GTP, and phosphorylated-ERK status (n≤3). (FIG. 2G) Representative images show TMR-dextran positive macropinosomes (arrowheads) in Ras^{V12} prostatic mouse fibroblasts (expressing GFP) compared to its WT counterpart. (FIG. 2H) Schematic illustrates orthotopic tissue recombinant xenograft models of CWR22Rv1 and Ras^{V12} mouse fibroblast allowed to grow for two weeks, followed by treatment with vehicle or 10 mg/kg EIPA (n=8). Representative gross tumor images are shown with a graph of all the tumor volumes. P value was calculated using two tailed student's t test. Data are represented as mean±SEM. **P<0.01. Scale bars: 30 μm (B, C, E, G); scale bars: 3 mm (H).

FIG. 3A-3H shows that ras activation in CAF drives glutamine metabolism in epithelia. (FIG. 3A) Relative mRNA expression levels of CAF-marker genes in WT and Ras^{V12} mouse fibroblast determined by quantitative rtPCR and analyzed using 2-way ANOVA (n≤3). (FIG. 3B) EpCAM-positive cancer epithelial was quantitated by FACS of 3D co-cultures of human epithelial (CWR22Rv1) cells and WT or Ras^{V12} mouse fibroblasts. Statistical analysis was performed using two-tailed student's t test (n≤3). (FIG. 3C) ATP was measured in CWR22Rv1 after incubation with conditioned media human NAF or CAF or mouse WT or Ras^{V12} fibroblast or oligomycin in glutamine free media (n≤3). Statistical analysis was performed using 1-way ANOVA with multiple comparisons. (FIG. 3D) The diagram represents the TCA cycle in CWR22Rv1 cells treated with NAF- or CAF-conditioned media for 72 hours prior to metabolome analysis (n≤3). (FIG. 3E) The metabolome analysis further indicated differential flux to aspartate, oxidized GSH, and GSH-reduced in CWR22Rv1 cells incubated with NAF- or CAF-conditioned media. (FIG. 3F) Glutamine concentrations were measured in the conditioned media from indicated mouse fibroblasts cultured for 72 hours. (FIG. 3D-FIG. 3F) Statistical analysis was performed using two-tailed student's t test (n≤3). (FIG. 3G) CWR22Rv1 proliferation was measured by cell counting following incubation with NAF and CAF conditioned media for 72 hrs in glutamine free media. NAF conditioned media was supplemented with 0.4 mM glutamine to mimic the glutamine levels expressed by CAF (see FIG. 11B). Statistical analysis was performed using 1-way ANOVA with multiple comparisons (n≤3). (FIG. 3H) Proton magnetic resonance spectroscopy data was acquired from orthotopically xenografted mice before and after EIPA administration. Spectra of the unfiltered data are superimposed using simulated echo acquisition (n=3-4 per group). Glutamate, GLU; Glutamine, GLN; ppm, parts per million. *P<0.05, **P<0.01, ***P<0.001, and ****P<0.0001.

FIGS. 4A-4G shows that stromal derived-glutamine induces mitochondrial bioenergetics and induction of PCa neuroendocrine differentiation. (FIG. 4A) Kinetic oxygen consumption rate (OCR) response in CWR22Rv1 cells exposed to 2 mM glutamine and rotenone along with antamycin. The OCR values were normalized to total protein. (FIG. 4B) Bioenergetic parameters of CWR22Rv1 cells incubated with wild type (WT)- or Ras^{V12}-fibroblast-conditioned media in the presence or absence of GPNA. Left: OCR trace for all three groups. Right: The basal OCR, ATP and maximal respiration are separately represented. Representative image of 3 independent experiments is shown. (FIG. 4C) CWR22Rv1 were co-cultured with WT- and Ras^{V12}-mouse prostatic fibroblasts in 3D, treated with vehicle, BPTES, or GPNA. Box plot of epithelial proliferation was measured by FACS analysis of EpCAM⁺/Ki67⁺ cells. Statistical analysis was performed using two tailed student's t test. (FIG. 4D) Phosphorylated-mTOR and FOXM1 protein levels were measured in CWR22Rv1 cells incubated with either WT- or Ras^{V12}-conditioned media for 3 days. (FIG. 4A-FIG. 4D) Representative image of 3 independent experiments is shown. (FIG. 4E) STRING analysis of a PCa neuroendocrine gene signature demonstrates direct or indirect interactions with FOXM1. (FIG. 4F) Heatmap summarizing the qPCR results comparing the CWR22Rv1 expression of neuroendocrine marker genes following treatment with conditioned media from WT- or Ras^{V12}-fibroblasts or (FIG. 4G) L-glutamine alone in combination with rapamycin was compared to vehicle. Two-way ANOVA analysis indicates the overall P value with F-test P<0.0001 for both experiments (n=3; F, G). Epithelial and stromal cells were grown in glutamine free either DMEMF12 or RPMI media. Data are represented as mean±SEM. *P<0.05, ***P<0.001, and ****P<0.0001.

FIGS. 5A-5F shows that androgen-mediated epigenetic regulation of RASAL3 expression in CAF determines PCa neuroendocrine differentiation and tumor growth in castrate resistant xenograft models. (FIG. 5A) rtPCR analysis of RASAL3 mRNA expression in NAF and CAF was performed following 5-day treatment with bicalutamide (Bic, 10⁻⁵ M), enzalutamide (Enza, 10⁻⁵ M), R1881 (10⁻⁹ M), or 5-Azacytadine (5-Aza, 5 mmol/L). ß-actin was used as a loading control. Representative image of 3 independent experiments is shown. (FIG. 5B) Bisulfide sequencing of the RASAL3 exon 2 was performed on CAF following treatment with vehicle or R1881 for 5 days. The methylation status of the individual CpG dinucleotides is shown by unmethylated (empty) or methylated (filled) circles (n=5). (FIG. 5C) 3D co-cultures of human CWR22Rv1 with CAF were treated with vehicle, enzalutamide, BPTES, or GPNA for 3 days in glutamine free media and subjected to quantitative rtPCR for the expression of a neuroendocrine prostate cancer gene panel. Two-way ANOVA followed by Tukey's multiple comparisons test was performed to determine P value (n≥3). (FIG. 5D) Schematic diagram of the experimental design of castration, enzalutamide, and GPNA treatment of a castrate resistant xenograft model. Sub-renal capsule xenografts contained tissue recombinants of CWR22Rv1 and CAF. (FIG. 5E) Gross images of representative tumors (dashed circle) as shown on host kidneys. (FIG. 5F) Quantitated tumor volumes are represented as the mean±SEM, analyzed by two-tailed student's t test (n≥8). **P<0.01, ***P<0.001, and ****P<0.0001. FIG. 6A-B. Glutamine uptake promotes tumor neuroendocrine differentiation and survival in the context of ADT.

FIGS. 6A-6B shows that glutamine uptake promotes tumor neuroendocrine differentiation and survival in the context of ADT. (FIG. 6A) H&E and chromogranin A (CHGA) staining of PCa xenografts indicate regulation by glutamine uptake antagonist, GPNA. (FIG. 6B) Representative staining for phosphorylated-mTOR, phosphorylated-histone H3 and TUNEL are shown for the PCa xenografts. Quantification of positive staining demonstrated significant differences when mice were treated with GPNA and ADT, compared to ADT alone. Scale bar represents 50 μm (n≥8; A, B). Data represent the mean±SEM. Statistical analysis was performed using two tailed student's t test. *P<0.05 and ***P<0.001.

FIGS. 7A-7B shows that sensitivity to androgen deprivation therapy correlates with blood glutamine levels is in support of a model of epigenetic Ras activation in prostatic fibroblasts and glutamine-mediated paracrine activity on prostate cancer epithelia. (FIG. 7A) Waterfall plot of plasma glutamine concentration in PCa patients on ADT correlated to therapeutic response (n=28; see Table 1). Based on serum PSA detection, patients were determined to be responsive (green bars) and non-responsive (red bars) to ADT. The threshold of 2 mM glutamine was chosen as a determinant of ADT responsiveness. Fisher's exact test was used to determine odds ratio. (FIG. 7B) While RASAL3 promoter hypermethylation and gene silencing are observed in CAF compared to NAF, androgen targeted therapy furthers this epigenetic Ras activation process that induces macropinocytosis in stromal fibroblasts for the uptake of albumin. Albumin is degraded by the lysosomes to generate glutamine (Gln), shuttled into epithelia through glutamine transporter, SLC1A5. Glutamine is converted to glutamate (Glu) and α-ketoglutarate (αKG) in entering the TCA cycle in support of PCa epithelial proliferation. Glutamine also contributes to mTOR activation, leading to neuroendocrine differentiation (NED).

FIGS. 8A-8B show that methylome analysis revealed differential RASAL3 promoter methylation. (FIG. 8A) Volcano plot of methylated genes from the heat map shown in FIG. 1A was generated with a fold-change cutoff of 1.5 and a P-value cutoff of 0.05. (FIG. 8B) Representative image of methylation-specific PCR (MS-PCR) for exon 2 of the RASAL3 gene in primary human NAF and CAF. Each experiment was repeated 3 times.

FIGS. 9A-9C depict macropinocytosis of prostatic CAF and epithelia. (FIG. 9A) Primary CAF were treated with DNA de-methylating agent, 5-Aza DC (5 μM), or macropinocytosis inhibitor, EIPA (25 μM) Macropinocytosis was detected by fluorescent TMR-dextran labeling (red). (FIG. 9B) Acidification of macropinosomes were monitored by co-localization of Lysotracker (green) with TMR-Dextran suggesting fusion of macropinosomes with lysosomes. Images show representative confocal microscopic images. (FIG. 9C) TMR-dextran staining of PCa epithelia (CWR22Rv1 and C4-2B) suggest little to no uptake of the dye in macropinocytic vesicles. DAPI staining (blue) identifies nuclei. Each experiment was repeated 3 times. Scale bars: 30 μm.

FIGS. 10A-10C shows that fibroblastic macropinocytosis is regulated by RASAL3 and Ras signaling. (FIG. 10A) The viability of CWR22Rv1 and mouse prostatic fibroblasts (Cas9 control and Rasa13-KO) was measured by MTT assay following 72 hrs incubation with macropinocytotic inhibitor, EIPA. The bar graphs represent the absorbance of the MTT formazan determined at 570 nm from a single experiment representative of a total 3 independent experiments, each performed in quadruplicate. Statistical significance was determined by one-way ANOVA. (FIG. 10B) Quantitation of EpCAM-positive epithelial cells were achieved by FACS of 3D co-cultures of human C4-2B epithelia and wild type (WT) or Ras^{V12} mouse fibroblasts. Statistical significance was determined by unpaired t test. (FIG. 10C) Stromal fibroblasts (Cas9 or Rasa13-KO) cultured with CWR22Rv1 epithelia using a standard transwell apparatus. Co-culture of CWR22Rv1 cells with Rasa13-KO stromal fibroblast for 48 hrs resulted in increased cell proliferation compared to that with Cas9 (control) fibroblasts, as assessed by trypan blue cell counting. Epithelial and stromal cells grown in glutamine free either DMEMF12 or RPMI media. are expressed as mean±SEM of 3 independent experiments. Statistical significance was determined by two-tailed student's t test. **P<0.01, ****P<0.0001.

FIGS. 11A-11J show that stromal glutamine uptake and metabolism by CWR22Rv1 and C4-2B prostatic epithelia. (FIG. 11A) ATP was measured in C4-2B cells after incubation with conditioned media (CM) from mouse wild type (WT) or Ras^{V12} mouse prostatic fibroblasts or oligomycin. Data are represented as mean±SEM, with n=3-4 per group, analyzed by one-way ANOVA with multiple comparison. (FIG. 11B) Glutamine concentrations were measured in the conditioned media derived from NAF and CAF cultured for 72 hrs. Data were obtained from 3 independent measurements, and each experiment was performed in quadruplicate. Statistical significance was determined by two-tailed student's t test. (FIG. 11C) Relative gene expression levels of SLC1A5, in CWR22Rv1 and C4-2B cells were measured following incubation with NAF or CAF conditioned media (CM) for 72 hrs. Statistical significance was determined by one-way ANOVA, n=3-5 per group. (FIG. 11D-FIG. 11G) Relative gene expression levels of glutamine transporters (SLC1A5, SLC38A1, SLC38A2 and SLC38A4) were measured in CWR22Rv1 or C4-2B cells treated with glutamine (2 mM) at indicated time points. Data represent the mean±SEM, Statistical significance was determined by two-tailed student's t test. (FIG. 11H) Relative gene expression levels of glutaminase isoforms (GLS and GLS2) in C4-2B cells were measured after treating with glutamine (2 mM) at indicated time points. Data represent the mean±SEM was evaluated by one-way ANOVA followed by sidak multiple comparison. n=3-5 per group. (FIG. 11I-FIG. 11J) Relative gene expression levels of GLS and GLS2, in CWR22Rv1 and C4-2B cells were measured following incubation with NAF-CM or CAF-CM for 72 hrs. and stromal cells grown in glutamine free either DMEMF12 or RPMI media. Statistical significance was determined by one-way ANOVA, n=3-5 per group. *P<0.05, **P<0.01, ***P<0.001, and ****P<0.0001.

FIGS. 12A-12C show that stromal glutamine supports the energy needs and mediates neuroendocrine differentiation of PCa epithelia. (FIG. 12A) Oxygen consumption rate (OCR) trace for WT-CM and Ras^{V12}-CM-cultured C4-2B cells, measured using a Seahorse Bioscience XF24 Extracellular Flux Analyzer. The injection order of oligomycin, FCCP, and rotenone along with antamycin resulted in a profile, normalized to protein concentration (n=4). Data shown of single experiment representative of a total 3 independent experiments, each performed in quadruplicate. The mean and S.D. of the three independent OCR experiments are represented by the bar graph highlighting basal, ATP-linked and maximal respiration elevated by Ras^{V12}-CM treated C4-2B cells including, compared to WT-CM conditioned media. Statistical significance was determined by two-tailed student's t test. ***P<0.001, and ****P<0.0001. (FIG. 12B) Heatmap summarizing the qPCR results comparing the C4-2B cell expression of neuroendocrine marker genes following treatment with L-glutamine (2 mM). Two-way ANOVA analysis indicated the overall P value with F-test P<0.0001 (n=3). (FIG. 12C) Following transfection with either GLS or SLC1A5 siRNA or scrambled control siRNA, CWR22Rv1 cells were incubated with 2 mM L-glutamine for measurement of mRNA levels of select neuroendocrine genes, assessed by qRT-PCR. Results are the average of at least three independent experiments. (FIG. 12B-FIG. 12C) Epithelial and stromal cells grown in glutamine free either DMEMF12 or RPMI media. Statistical significance is summarized in Table 5.

FIGS. 13A-13B show that RASAL3 expression and promoter methylation is androgen dependent. (FIG. 13A) Quantitative rtPCR analysis of RASAL3 in NAF and CAF was performed following 5 day treatment with bicalutamide (Bic, 10⁻⁵ M), enzalutamide (Enza, 10⁻⁵ M), R1881 (10⁻⁹ M), or 5-Azacytidine (5-Aza, 5 mmol/L). RASAL3 expression was normalized GAPDH expression. Data represent the mean±SEM. *P<0.05, ***P<0.001, and ****P<0.0001, by two-way ANOVA followed by multiple comparison. Results are the average of at least three independent experiments. (FIG. 13B) MS-PCR of the RASAL3 exon 2 methylation status in CAF after treatment with enzalutamide, R1881, 5-Azacytidine and bicalutamide was performed. The PCR products in the lanes marked “U” indicate unmethylated templates for RASAL3 gene, whereas the products in the lanes marked “M” indicate methylated templates. Data are representative of three independent experiments.

FIG. 14A-14D depict GPNA treatment of intact mice with tissue recombinant xenografts of PCa epithelia and CAF. (FIG. 14A) Schematic diagram of the experimental design of control or GPNA treatment of mice xenografted with tissue recombinants in the sub-renal capsule consisted of CWR22Rv1 (2×10⁵ cells) and CAF (6×10⁵ cells). (FIG. 14B) Quantitated tumor volumes were analyzed by two-tailed t test (n=4). (FIG. 14C) H&E and phosphorylated histone H3 (p-histone H3) immunohistochemical staining of PCa xenografts indicate regulation by glutamine uptake antagonist, GPNA. Experiment was performed 3 separate times. Representative image of 3 independent experiments is shown. (FIG. 14D) Quantification of the frequencies of p-histone H3-positive cells per 20× field are represented as a mean SEM was evaluated by two tailed Student's t test. ***P<0.001, ****P<0.0001.

FIGS. 15A-E depict (FIG. 15A) Bone morphogenic protein (BMP) expression is upregulated by enzalutamide and glutamine (Gln) in human prostate cancer cells (CWR22Rv1). (FIG. 15B) Increasing doses of Gln upregulates osteopotegrin (OPG) and FoxMl (pro-metastatic gene expression). (FIG. 15C) Gln upregulates TET2 (chromatin modifying protein) associated with tumor progression. (FIG. 15D) Enzalutamide induces TET2 expression and inhibition of glutaminase (BPTES) downregulates enzalutamide-induced TET2. (FIG. 15E) Enzalutamide promotes glutamine expression in bone marrow fibroblasts and M1043 (inhibition of CD105) downregulates glutamine expression.

FIGS. 16A-16B depict the expression of immune checkpoint protein inhibitors of T cells (PD1) and macrophage (CD47) by FACS analysis in a model of co-cultured prostate cancer with associated fibroblasts. Prostate cancer cells (CWR22Rv1) expression of PD1 and CD47 is downregulated with glutaminase inhibition with BPTES in the context of wild type fibroblasts and when in the context of CD105-knockout fibroblasts.

FIGS. 17A-17D depicts embodiments of the present invention. (FIG. 17A) Glutamine (Gln) upregulates ALDH (stem marker). But, glutamate upregulates ALDH further in prostate cancer epithelial cells. (FIG. 17B) Enzalutamide upregulates ALDH, that can be limited by glutaminase inhibition (BPTES), but not CD105 inhibition (1′RC105). This is thought to be the case since the TRC105 acts on the stromal fibroblasts (absent in epithelial culture). (FIG. 17C) In prostatic fibroblasts, enzalutamide promotes glutamine expression in the conditioned media that is downregulated by CD105 inhibition (M1043). (FIG. 17D) Similarly, bone marrow fibroblasts expressed elevated glutamine that was downregulated by CD105 inhibition (M1043).

FIGS. 18A-18B show that CD105 inhibition limited colon cancer metastasis in a model of liver metastasis. In a model of colon cancer liver metastasis, CD105 (endoglin) inhibition (M1043) limited tumor number and tumor size (FIG. 18A) in the liver (FIG. 18B).

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this present disclosure belongs. Allen et al., Remington: The Science and Practice of Pharmacy 22nd ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3rd ed., revised ed., J. Wiley & Sons (New York, N.Y. 2006); Smith, March's Advanced Organic Chemistry Reactions, Mechanisms and Structure 7th ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application.

All publications cited herein are incorporated by reference in their entirety to the same extent as if each individual publication or patent application was specifically and individually indicated to be incorporated by reference. The following description includes information that may be useful in understanding the present disclosure. It is not an admission that any of the information provided herein is prior art or relevant to the presently claimed present disclosure, or that any publication specifically or implicitly referenced is prior art.

One skilled in the art will recognize many methods and materials similar or equivalent to those described herein, which could be used in the practice of the present disclosure. Other features and advantages of the present disclosure will become apparent from the following detailed description, taken in conjunction with the accompanying drawings, which illustrate, by way of example, various features of embodiments of the present disclosure. Indeed, the present disclosure is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention, because the scope of the invention is limited only by the claims.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

Unless stated otherwise, the terms “a” and “an” and “the” and similar references used in the context of describing a particular embodiment of the application (especially in the context of claims) may be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein may be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

“Optional” or “optionally” means that the subsequently described circumstance may or may not occur, so that the description includes instances where the circumstance occurs and instances where it does not.

Groupings of alternative elements or embodiments of the present disclosure disclosed herein are not to be construed as limitations. Each group member may be referred to and claimed individually or in any combination with other members of the group or other elements found herein. One or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

As used herein, the terms “treat,” “treatment,” “treating,” or “amelioration” when used in reference to a disease, disorder or medical condition, refer to therapeutic treatment wherein the object is to reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease, disorder or medical condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented. Non-limiting examples of treatments or therapeutic treatments include at least one selected from pharmacological therapies, biological therapies, interventional surgical treatments, and combinations thereof.

“Beneficial results” or “desired results” may include, but are in no way limited to, lessening or alleviating the severity of the disease or condition, preventing the disease or condition from worsening, curing the disease or condition, preventing the disease or condition from developing, lowering the chances of a patient developing the disease or condition, decreasing morbidity and mortality, and prolonging a patient's life or life expectancy. As non-limiting examples, “beneficial results” or “desired results” may be alleviation of one or more symptom(s), diminishment of extent of the deficit, stabilized (i.e., not worsening) state of a disease, delay or slowing of a disease, and amelioration or palliation of symptoms associated with a disease.

The term “condition” when referring to a subject is understood in the present disclosure as status of subject that may be described by physical, mental or social criteria. It includes as well so-called “healthy” and “diseased” conditions, therefore it is not limited to the WHO definition of health as “a state of complete physical, mental, and social well-being and not merely the absence of disease or infirmity.” but includes disease and infirmity.

The term “disease” refers to an abnormal condition affecting the body of an organism. The term “disorder” refers to a functional abnormality or disturbance. The terms disease or disorder are used interchangeably herein unless otherwise noted or clear given the context in which the term is used.

Non-limiting examples of diseases and conditions and disease conditions and medical conditions include cancer, tumor, liver fibrosis, and pancreatic fibrosis.

A “cancer” or “tumor” as used herein refers to an uncontrolled growth of cells which interferes with the normal functioning of the bodily organs and systems, and/or all neoplastic cell growth and proliferation, whether malignant or benign, and all pre-cancerous and cancerous cells and tissues. A subject that has a cancer or a tumor is a subject having objectively measurable cancer cells present in the subject's body. Included in this definition are benign and malignant cancers, as well as dormant tumors or micrometastasis. Cancers which migrate from their original location and seed vital organs can eventually lead to the death of the subject through the functional deterioration of the affected organs. As used herein, the term “invasive” refers to the ability to infiltrate and destroy surrounding tissue. In some embodiments, the tumor is a solid tumor.

In various embodiments, the condition is a cancer. In some embodiments, the condition is a disease. In some embodiments, the disease is a cancer. Examples of cancer include but are not limited to breast cancer such as a ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; cervical cancers such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; acute myeloid leukemia (AML), preferably acute promyleocytic leukemia in peripheral blood; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's sarcoma; Ewing sarcoma; central nervous system cancers such as primary brain tumor, which includes gliomas (astrocytoma, anaplastic astrocytoma, or glioblastoma multiforme (GBM)), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas; oral cavity and oropharyngeal cancer; stomach cancer such as lymphomas, gastric stromal tumors, and carcinoid tumors; testicular cancer such as germ cell tumors (GCTs), which include seminomas and nonseminomas; and gonadal stromal tumors, which include Leydig cell tumors and Sertoli cell tumors; head cancer; neck cancer; throat cancer; and thymus cancer, such as to thymomas, thymic carcinomas, Hodgkin disease, non-Hodgkin lymphomas carcinoids or carcinoid tumors. Also, the methods may be used to treat viral-induced cancers. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer.

In various embodiments, the cancer may include a Ras mutated cancer. In various embodiments, the cancer may include Ras signaling activation through epigenetic influence; for example, DNA methylation or acetylation, histone methylation or acetylation, microRNA and other non-coding RNA. In various embodiments, the cancer may be selected from the group comprising: adenocarcinoma, neuroendocrine cancer, and combinations thereof.

A “healthy subject” or “normal subject” is a subject (e.g., patient) who does not have the disease or disorder that is being treated in the subject in need thereof.

As used herein, the term “administering,” refers to the placement an agent as disclosed herein into a subject by a method or route which results in at least partial localization of the agents at a desired site. “Route of administration” may refer to any administration pathway known in the art, including but not limited to aerosol, nasal, via inhalation, oral, anal, intra-anal, peri-anal, transmucosal, transdermal, parenteral, enteral, topical or local. “Parenteral” refers to a route of administration that is generally associated with injection, including intratumoral, intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions may be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions may be in the form of aerosol, lotion, cream, gel, ointment, suspensions, solutions or emulsions. In accordance with the present disclosure, “administering” may be self-administering. For example, it is considered as “administering” that a subject consumes a composition as disclosed herein.

“Diagnostic” means identifying the presence or nature of a pathologic condition, disease, or disorder and includes identifying patients who are at risk of developing a specific condition, disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, a disease, or a disorder, it suffices if the method provides a positive indication that aids in diagnosis.

By “at risk of” is intended to mean at increased risk of, compared to a normal subject, or compared to a control group, e.g. a patient population. Thus a subject carrying a particular marker may have an increased risk for a specific condition, disease or disorder, and be identified as needing further testing. “Increased risk” or “elevated risk” mean any statistically significant increase in the probability, e.g., that the subject has the disorder. In some embodiments the risk is increased by at least 10% over the control group with which the comparison is being made. In some embodiments, the risk is increased by at least 20% over the control group with which the comparison is being made. In some embodiments, the risk is increased by at least 50% over the control group with which the comparison is being made.

The term “diagnosis,” or “dx,” refers to the identification of the nature and cause of a certain phenomenon. As used herein, a diagnosis typically refers to a medical diagnosis, which is the process of determining which disease or condition explains a symptoms and signs. A diagnostic procedure, often a diagnostic test or assay, may be used to provide a diagnosis. A diagnosis can comprise detecting the presence of a disease or disorder, or condition.

The term “prognosis,” or “px,” as used herein refers to predicting the likely outcome of a current standing. For example, a prognosis can include the expected duration and course of a disease or disorder, such as progressive decline or expected recovery.

“Sample” is used herein in its broadest sense. The term “biological sample” as used herein denotes a sample taken or isolated from a biological organism. Exemplary biological samples include, but are not limited to, cheek swab; mucus; whole blood, blood, serum; plasma; urine; saliva; semen; lymph; fecal extract; sputum; other body fluid or biofluid; cell sample; and tissue sample etc. The term also includes a mixture of the above-mentioned samples. The term “sample” also includes untreated or pretreated (or pre-processed) biological samples. In some embodiments, a sample can comprise one or more cells from the subject. In some embodiments, a sample is a tissue or tissue sample. In some embodiments, the sample is a tumor or tumor sample or tumor tissue.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal may be a human, non-human primate, mouse, rat, dog, cat, horse, or cow, but are not limited to these examples. In addition, the methods described herein may be used to treat domesticated animals and/or pets. In some embodiments, the subject is a human. In some embodiments, the subject is a male subject. In some embodiments, the subject has a cancer. In some embodiments, the subject has a tumor.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

A subject may be one who has been previously diagnosed with or identified as suffering from or having a disease, disorder or condition in need of treatment or one or more complications related to the disease, disorder, or condition, and optionally, have already undergone treatment for the disease, disorder, or condition or the one or more complications related to the disease, disorder, or condition. Alternatively, a subject can also be one who has not been previously diagnosed as having a disease, disorder, or condition or one or more complications related to the disease, disorder, or condition. For example, a subject may be one who exhibits one or more risk factors for a disease, disorder, or condition or one or more complications related to the disease, disorder, or condition or a subject who does not exhibit risk factors. A “subject in need” of treatment for a particular disease, disorder, or condition may be a subject suspected of having that disease, disorder, or condition, diagnosed as having that disease, disorder, or condition, already treated or being treated for that disease, disorder, or condition, not treated for that disease, disorder, or condition, or at risk of developing that disease, disorder, or condition.

The term “statistically significant” or “significantly” refers to statistical evidence that there is a difference. It is defined as the probability of making a decision to reject the null hypothesis when the null hypothesis is actually true. The decision is often made using the p-value.

The terms “marker” or “biomarker” are used interchangeably herein, and in the context of the present disclosure refers to a composition, substance, compound, chemical, agent, or metabolite or derivative thereof associated with a disease, disorder, or condition wherein the marker is differentially present in a sample taken from patients having a specific disease, disorder, or condition as compared to a control value, the control value includes, for example average or mean values in comparable samples taken from control subjects (e.g., a person with a negative diagnosis, normal or healthy subject).

In some embodiments, the marker is glutamine and/or at least one glutamine metabolite. Non-limiting examples of glutamine metabolites include glutamate, glutathione, γ-aminobutyrate, α-ketoglutarate, succinate, fumarate, malate, acetyl-CoA, 2-hydroxyglutarate and combinations thereof. In some embodiments, biomarkers may be determined as glutamine and/or at least one glutamine metabolite which may be detected by sample assay and/or spectral magnetic resonance imaging. In some applications, for example, spectral magnetic resonance images and or sample assay results may be used to determine multiple biomarkers, and differences between individual biomarkers and/or the partial or complete profile may be used for diagnosis.

A “test amount” of a marker refers to an amount of a marker present in a sample being tested. A test amount may be either in absolute amount (e.g., ug/ml) or a relative amount (e.g., relative intensity of signals).

A “diagnostic amount” of a marker refers to an amount of a marker in a subject's sample that is consistent with a diagnosis or a particular disease, disorder, or condition. A diagnostic amount may be either in absolute amount (e.g., ug/ml) or a relative amount (e.g., relative intensity of signals).

A “control amount” of a marker may be any amount or a range of amount which is to be compared against a test amount of a marker. For example, a control amount of a marker may be the amount of a marker in a person who does not suffer from the disease, disorder, or condition sought to be diagnosed. In another example, a control amount of a marker may be the amount of a housekeeping gene. A control amount may be either in absolute amount (e.g., ug/ml) or a relative amount (e.g., relative intensity of signals).

The term “differentially present” refers to differences in the quantity and/or frequency of a marker present in a sample taken from patients having a specific disease, disorder, or condition as compared to a control subject. For example, a marker may be present at an elevated level (e.g., increased level) or at a decreased level in samples of patients with the disease, disorder, or condition compared to a control value (e.g., determined from samples of control subjects). Alternatively, a marker may be detected at a higher frequency or at a lower frequency in samples of patients compared to samples of control subjects. A marker may be differentially present in terms of quantity, frequency or both as well as a ratio of differences between two or more specific modified amino acid residues and/or enzyme itself. In one embodiment, an increase in the amount or level of glutamine and/or at least one glutamine metabolite (e.g., glutamate) is diagnostic of any one or more of the diseases, disorders, or conditions described herein. In one embodiment, a decrease in the amount or level of glutamine and/or at least one glutamine metabolite (e.g., glutamate) is diagnostic of any one or more of the diseases, disorders, or conditions described herein.

A marker, compound, composition or substance is differentially present in a sample if the amount of the marker, compound, composition or substance in the sample is statistically significantly different from the amount of the marker, compound, composition or substance in another sample, or from a control value. For example, a marker is differentially present if it is present at least about 120%, at least about 130%, at least about 150%, at least about 180%, at least about 200%, at least about 300%, at least about 500%, at least about 700%, at least about 900%, or at least about 1000% greater or less than it is present in the other sample (e.g., control), or if it is detectable in one sample and not detectable in the other.

Alternatively, or additionally, a marker, compound, composition or substance is differentially present between samples if the frequency of detecting the marker, etc. in samples of patients suffering from a particular disease, disorder, or condition, is statistically significantly higher or lower than in the control samples or control values obtained from healthy individuals. For example, a biomarker is differentially present between two sets of samples if it is detected at least about 10%, at least about 20%, at least about 30%, at least about 40%, at least about 50%, at least about 60%, at least about 70%, at least about 80%, at least about 90%, or at least about 100% more frequently or less frequently observed in one set of samples than the other set of samples. These exemplary values notwithstanding, it is expected that a skilled practitioner can determine cut-off points, etc. that represent a statistically significant difference to determine whether the marker is differentially present.

Prostate cancer (PCa) is an androgen-dependent disease subject to interactions between the tumor epithelia and its microenvironment. Here, we found epigenetic changes to cancer-associated prostatic fibroblasts (CAF) initiated a cascade of stromal-epithelial interactions that allowed lethal prostate cancer to grow and further develop resistance to androgen deprivation therapy (ADT). We identified that a RAS-GAP gene, RASAL3, is epigenetically silenced in CAF. The resulting increase in oncogenic RAS downstream activity in CAF drove macropinocytosis-mediated glutamine synthesis. Interestingly, ADT further promoted RASAL3 epigenetic silencing and glutamine secretion in prostatic fibroblasts. In a mouse orthotopic xenograft model, subsequent inhibition of macropinocytosis and glutamine transport resulted in antitumor effects and ADT sensitivity. Moreover, stromal glutamine uptake served as a source of energy through anaplerosis of the TCA cycle as well as a neuroendocrine differentiation mediator for prostate adenocarcinoma. In validating these findings, we found that prostate cancer patients on ADT with therapeutic resistance had elevated blood glutamine levels compared to those with therapeutically responsive disease (odds ratio=7.451, p value=0.02). Identification of epigenetic regulation of RAS activity in prostatic CAF revealed RASAL3 as a sensor for metabolic and neuroendocrine reprogramming in prostate cancer patients failing ADT.

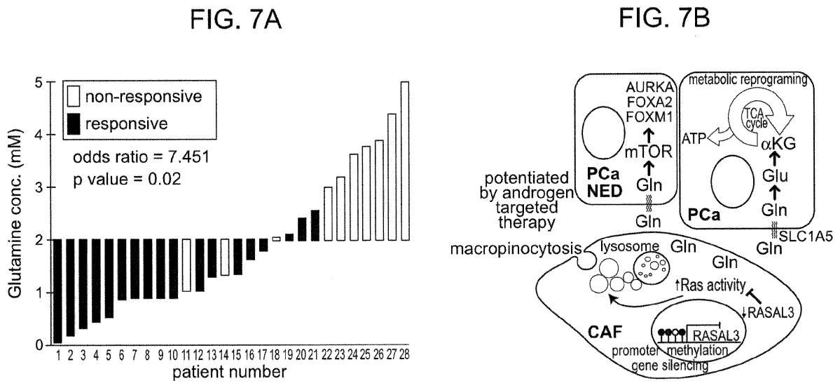

This study reports that ADT can induce epigenetic alteration of the stromal fibroblasts to promote prostate cancer (PCa) progression to a more aggressive differentiation state. We performed a whole genome methylome analysis to reveal the silencing of a tumor suppressor, RASAL3. The outcomes of the present interrogation of stromal-epithelial interactions are summarized in, e.g., FIG. 7, where the hypermethylation of the RASAL3 promoter in CAF induced Ras-dependent macropinocytosis for the lysosomal catabolism of albumin and generation of glutamine. Metabolomic profiling uncovered the role of stromal glutamine as an underlying metabolic controller facilitating cell survival and proliferation. Glutamine transporter facilitated transfer of stromal glutamine into epithelia for further metabolism to glutamate and α-ketoglutarate for utilization by the TCA cycle and mTOR activation, contributing to the energy needs of the cancer and its differentiation, respectively. This is the first demonstration of epigenetic Ras regulation in CAF; its consequence for PCa is its contribution to tumor progression and castrate responsivity.

We demonstrated that human CAFs have low expression of RASAL3, due to exon 2 hypermethylation, resulting in Ras signaling activation. We further found that elevated Ras activity in prostatic mouse wild type fibroblasts was sufficient to promote differentiation state analogous to CAF, defined to be tumor promoting. The present findings provide evidence for RasGAP gene methylation and RAS signaling in CAF. Silencing of RASAL3 contributed to downstream cRaf and ERK activation in the absence of a change in Ras expression. So, the common loss of PTEN expression in PCa epithelia, is not necessarily the only determinant for elevated PI3 kinase activity in tumor tissues.

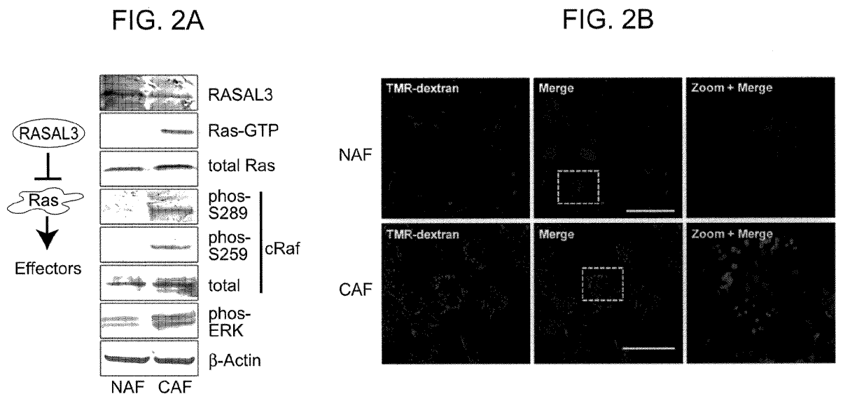

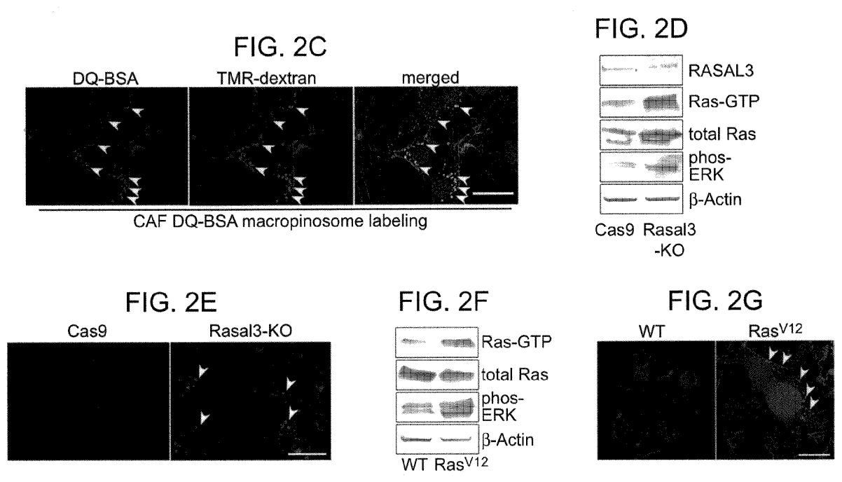

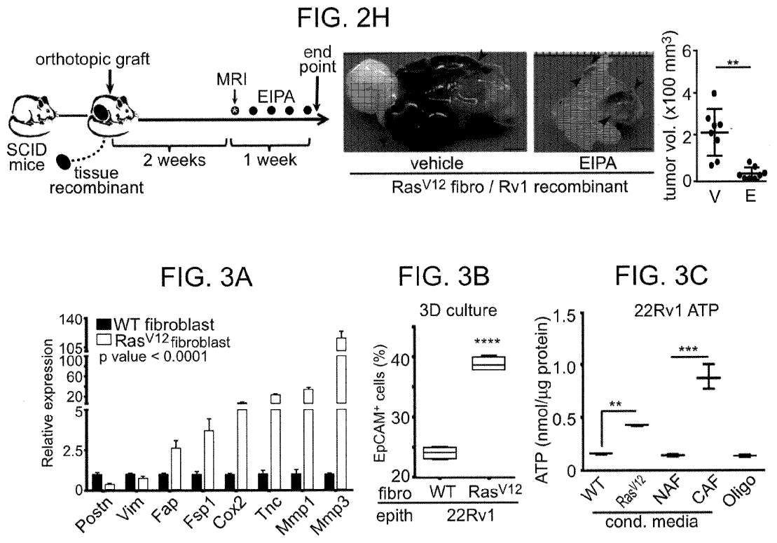

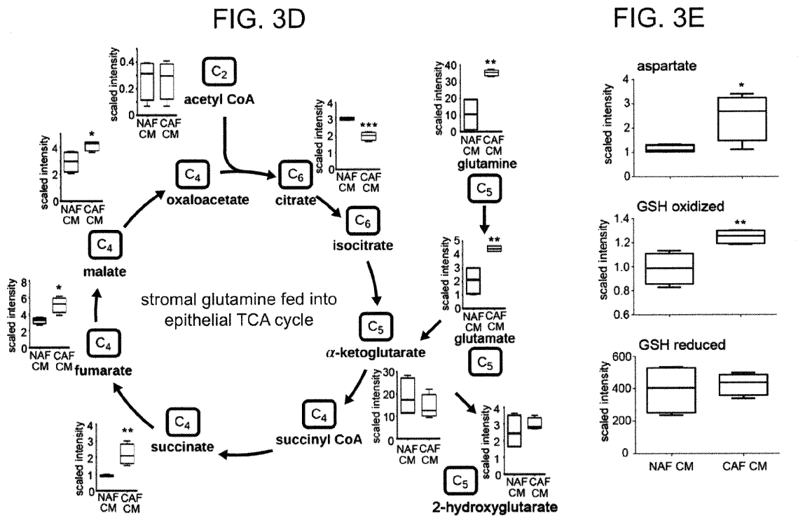

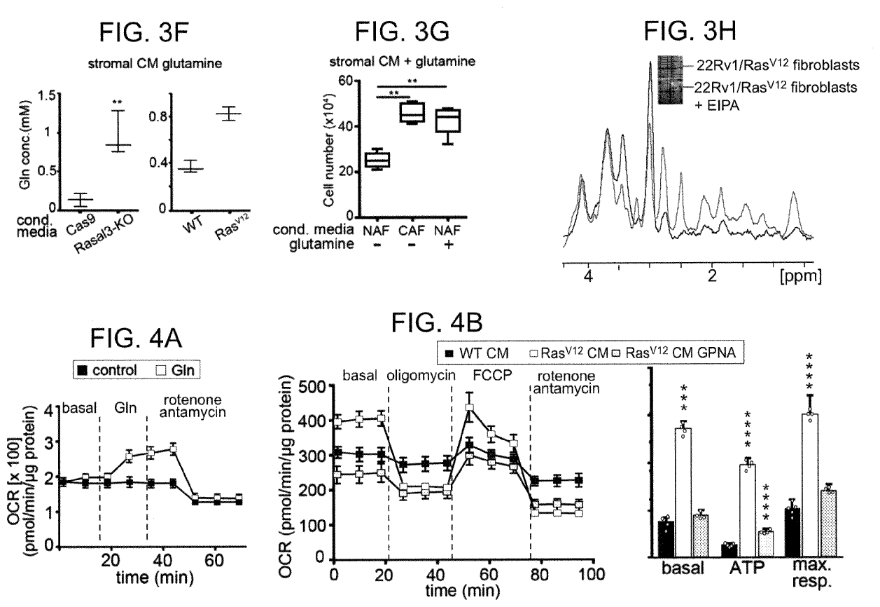

The seminal identification of the cellular process of macropinocytosis resulting from Ras signaling in pancreatic cancer revealed a metabolic strategy for addressing cancers with genetic alterations in this signaling axis. The overexpression of Ras^{V12} in wild type mouse prostatic fibroblasts recapitulated the observation of macropinocytosis, strikingly prevalent in primary human prostatic CAF derived from multiple patient tissues (see, e.g., FIGS. 2A-2H). While reminiscent of macropinocytosis identified in pancreatic cancer in terms of its support of adjacent cancer epithelia, the activation of Ras in prostatic fibroblasts demonstrated elevated expression of a gene panel associated with a reactive fibroblastic state. What was surprising was that androgen deprivation further silenced RASAL3 in NAF and was coincident with acquisition of CAF features (see, e.g., FIGS. 3A-3D). Thus, epigenetic modification of tumor associate fibroblasts may be initiated by ADT, and extraordinarily be the paracrine mechanism of resistance to ADT through a paracrine manner.

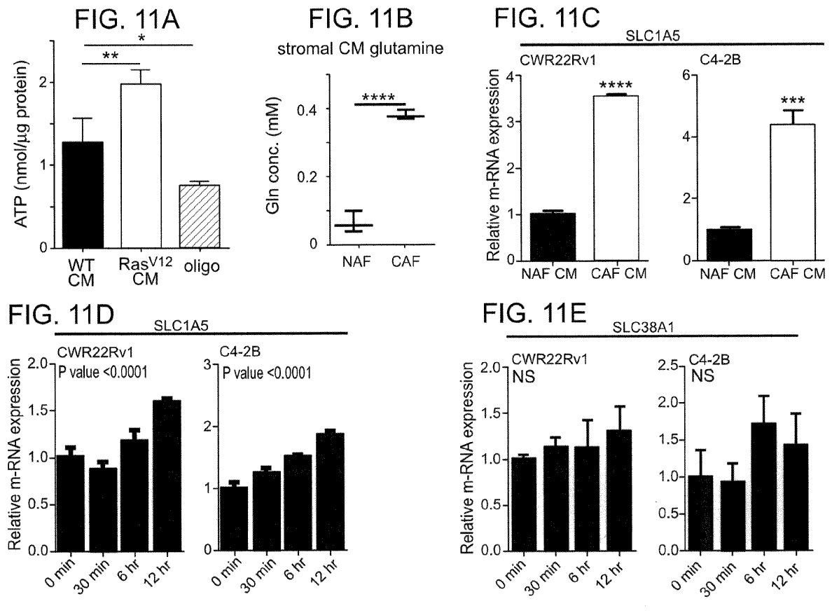

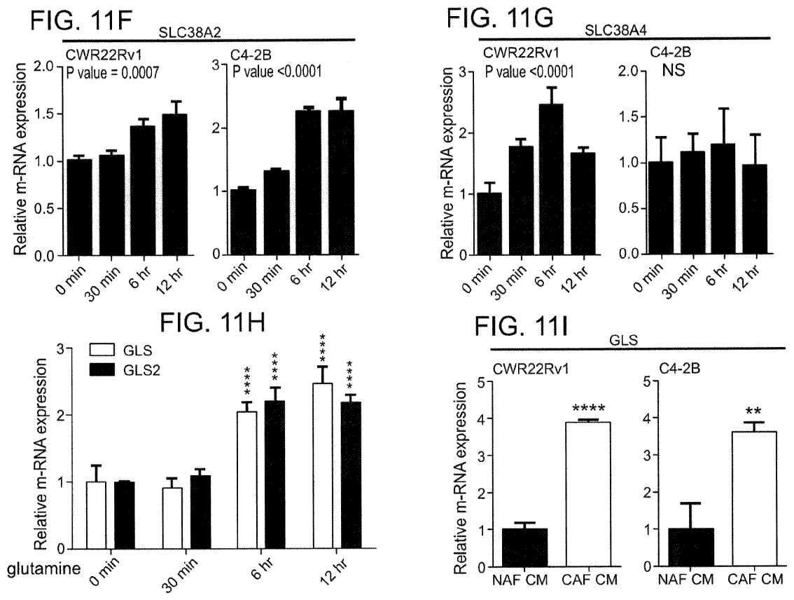

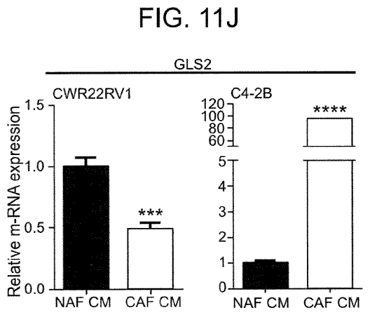

Cancer cells survive and even thrive under hypoxic and in what is thought to be nutrient deprived conditions. To fuel its high energy and biosynthetic needs lactate and ketone bodies secreted by CAF are used by adjacent epithelial cancer cells. Glutamine, a “conditionally essential” amino acid is the most commonly depleted amino acid in solid tumors, can serve as a carbon source for TCA cycle and as a nitrogen source for nucleotide biosynthesis. Like glucose, amino acids cannot cross the lipid bilayer without transporter proteins, and amino acid transporter expression is positively correlated with growth and cancer. The expression level of glutamine transporters and glutamine conversion to glutamate in CWR22RV1 and C4-2B cells are potentiated by glutamine secreted by CAF, allowing its own uptake (see, e.g., FIGS. 11A-11B). Within the epithelia, CAF-derived glutamine conversion to glutamate could be metabolized into α-ketoglutarate, aspartate, and glutathione (see, e.g., FIGS. 3A-3D). The lack of observed α-ketoglutarate accumulation was likely due to its oxidation to succinate, fumarate, and malate to address the energy needs by the TCA cycle. However, glutamate conversion to oxidized glutathione supported the accumulated oxidative stress mediated by the mitochondrial electron transport. Thus, the androgen-dependent switch of the tumor microenvironment to regulate glutamine is through RASAL3 epigenetic silencing. More practically, therapeutic approaches targeting hormone signaling must consider these metabolic adaptations.

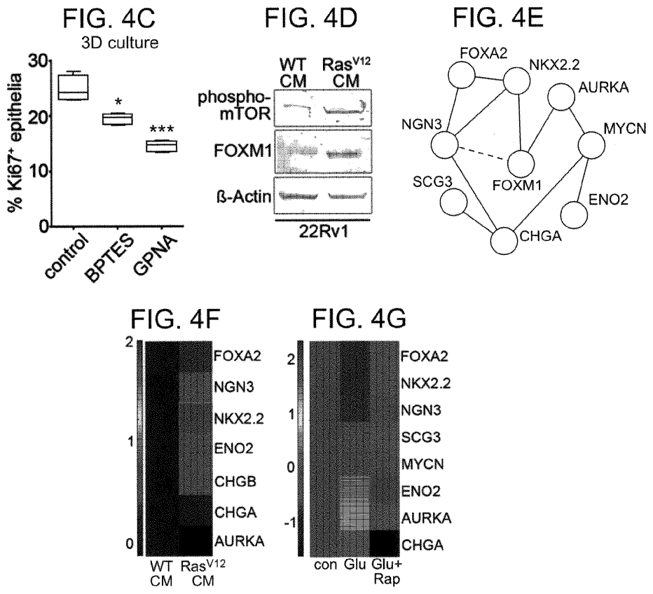

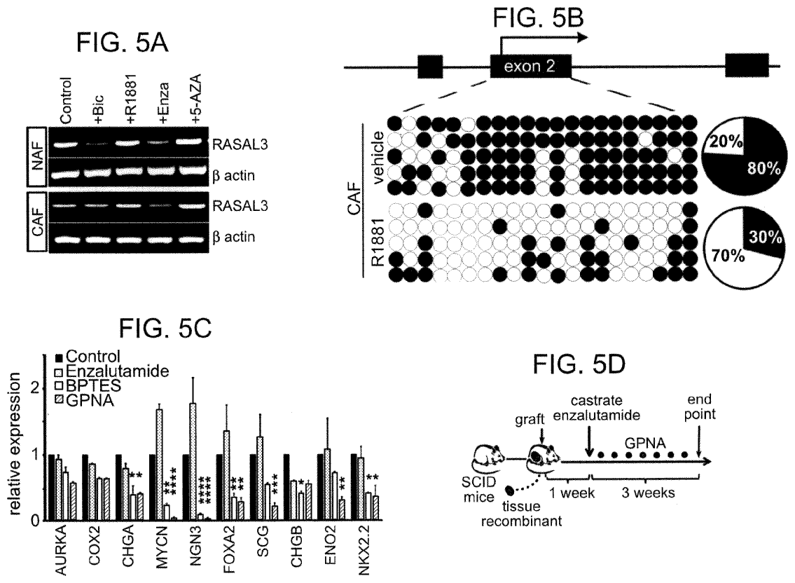

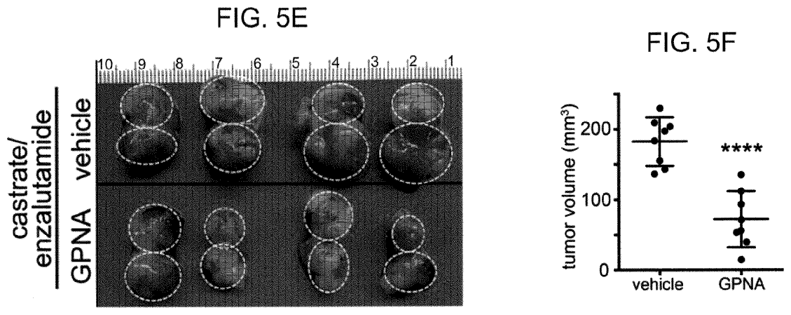

The precedence of PCa neuroendocrine differentiation associated with the ADT administration is a described resistance mechanism. However, mechanisms for ADT induction of neuroendocrine PCa are less well understood. An example involving paracrine IL-8 derived from neuroendocrine PCa cells was found to further promote neuroendocrine differentiation of PCa. Here we described an androgen-dependent mechanism for CAF-mediated neuroendocrine differentiation of adjacent prostatic epithelia. It is known that glutamine sensing triggers mTOR activity. MTOR signaling is associated with PCa neuroendocrine differentiation. We reasoned that targeting glutamine metabolism may serve as a therapeutic means of limiting neuroendocrine differentiation, in sensitizing PCa to ADT. Importantly, stromal glutamine was necessary and sufficient to promote PCa neuroendocrine differentiation (see, e.g., FIGS. 4A-4G, FIGS. 5A-5F). As ADT potentiated stromal glutamine secretion, combination therapy of castration/enzalutamide and GPNA, targeting glutamine uptake by cancer cells could serve as a viable synthetic lethal approach. The present finding showed that blocking glutamine transporter by GPNA or glutamine conversion by BPTES, helped reduce the neuroendocrine differentiation in PCa (see, e.g., FIGS. 4A-4G). This observation was further corroborated in vivo where pharmacological inhibition of SLC1A5 by GPNA, in the context of ADT significantly reduced tumor size compared to ADT alone in a model of castrate resistant disease (see, e.g., FIGS. 5A-5F). Previous findings supported SLC1A5 to be highly expressed in PCa and breast cancer tissues and their inhibition led to reduced cell growth. However, as this was the first demonstration of such a direct role the tumor microenvironment on promoting PCa neuroendocrine differentiation, the findings were validated in PCa patient blood samples. The data suggested that plasma glutamine may be used as a prognostic marker to follow ADT response and development of resistance (see, e.g., FIG. 7A).

The present disclosure provides a novel mechanism for ADT induced PCa neuroendocrine differentiation and further clinical validation through the measurement of plasma glutamine. Further, ADT may induce epigenetic alteration of the stromal fibroblasts to promote PCa neuroendocrine differentiation in glutamine dependent manner. The plasma glutamine concentration differences, the differentiate therapeutic responses in this small sample size was observed. The present study provides evidence that glutamine can be used as a prognostic marker to monitor ADT response.

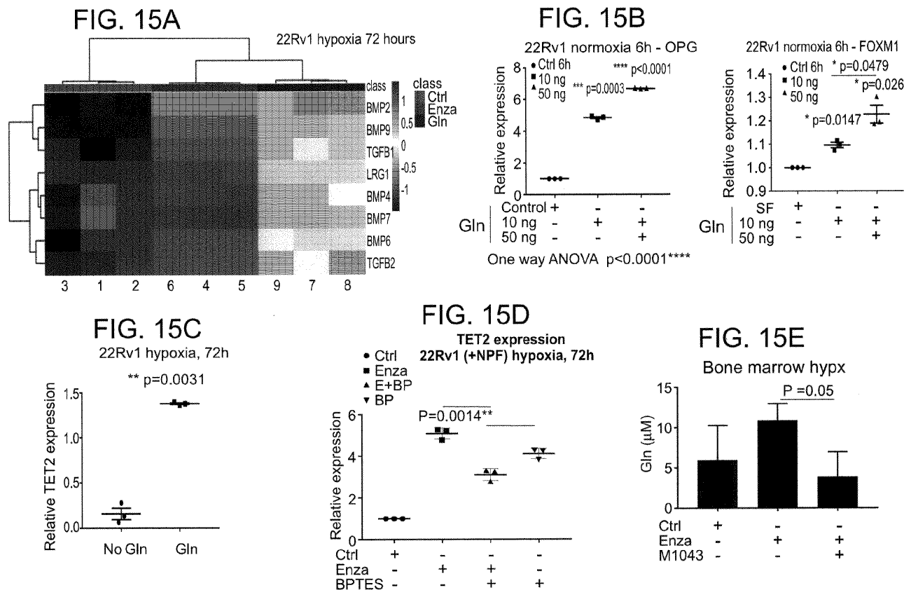

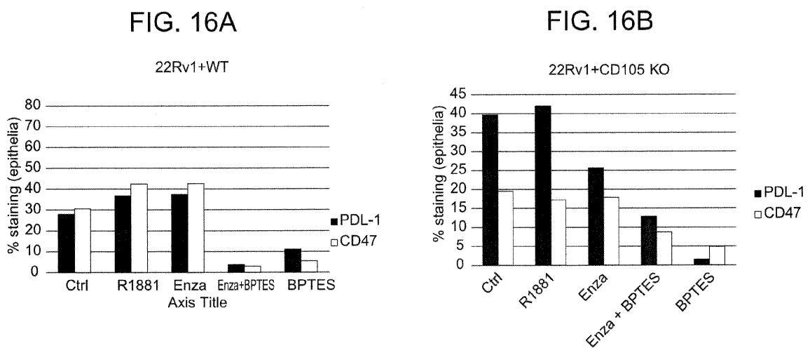

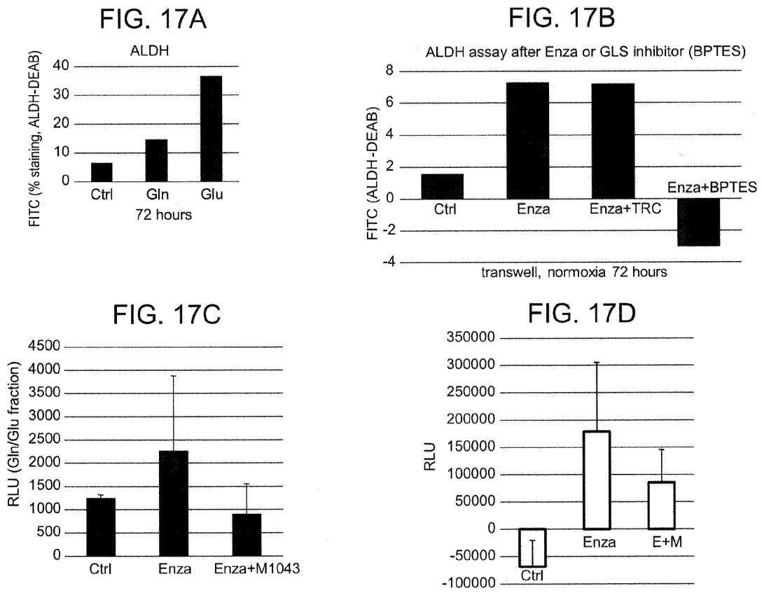

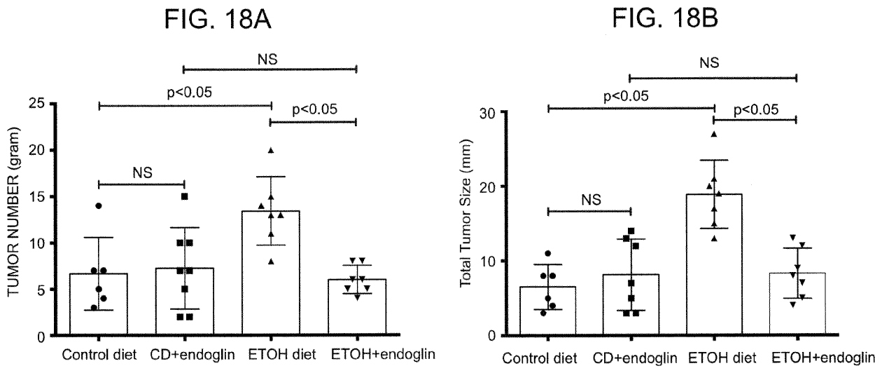

The inventors also found that androgen targeted therapies and glutamine upregulate bone morphogenic protein expression and BPTES (glutaminase inhibitor) and M1043 (a CD105 inhibitor) both downregulate glutamine, which is translatable to cancers that metastasize to the bone (FIG. 15). The inventors further found that CD105 inhibitors can also act as glutaminase inhibitors, and when used in combination of an androgen targeted therapy, downregulate glutamine and glutamate, limiting metastatic progression and therapeutic resistance (FIGS. 15 and 17). The inventors also found that CD105 antagonism can limit colon cancer metastasis to the liver, which is related to the decrease expression of glutaminase synthetase (FIG. 18). Furthermore, the inventors also discovered that glutaminase and CD105 inhibitors downregulate PD-L1 and CD47 (immune suppressors proteins). Therefore\, glutaminase inhibitors can be used in combination with immune checkpoint inhibitors or other complementary therapeutics to more effectively treat cancer.

Various embodiments of the present invention are based, at least in part, on these finding.

In various embodiments, the present disclosure provides that antagonizing macropinocytosis, glutamine uptake, or glutaminase activity can inhibit neuroendocrine differentiation of adenocarcinoma. Macropinocytosis is a means of albumin uptake, subsequent breakdown in the lysosomes, resulting in glutamine production. The differentiation of adenocarcinomas to a neuroendocrine phenotype is associated with therapeutic resistance to many conventional therapies. Prostate cancer is one such adenocarcinoma type that undergoes such differentiation in response to glutamine. There are many neuroendocrine cancers, such as small lung cancer and neuroendocrine pancreatic cancer, that have elevated mTOR activity and may be responsive to antagonizing macropinocytosis, glutamine uptake, or glutaminase activity. Further, such inhibitors may be used in combination with conventional chemotherapy (e.g., 5-FU, cisplatin, taxanes) to support a durable therapeutic effect.

The present disclosure provides an androgen signaling antagonist-therapy (e.g., androgen deprivation therapy (ADT)) that is associated with elevated glutamine secretion due to epigenetic changes by prostate cancer associated fibroblastic cells. Elevated glutamine was found to be a pro-survival factor for the tumor by providing an energy source and mediator of differentiation for the development of resistance to androgen signaling antagonist-therapy.

The present disclosure provides an androgen receptor signaling inhibitor that promote glutamine generation by prostate cancer associated fibroblastic cells through silencing of the RASAL3 gene. The glutamine generation by the fibroblastic cells was found to be a direct mediator of resistance to androgen receptor signaling inhibitors. Thus, in various embodiments, the present disclosure provides that antagonizing macropinocytosis, glutamine uptake, or glutaminase activity can sensitize prostate cancer to androgen receptor signaling inhibitors.

In various embodiments, the present disclosure provides that glutaminase activity contributes to radiation resistance. Accordingly, antagonizing macropinocytosis, glutamine uptake, or glutaminase activity will help to sensitize cancer to radiation therapy or enable the use of a lower dose of radiation therapy (mitigating unwanted side effects). Apart from the elevated energy needs of irradiated cancer cells, radiation requires the generation of reactive oxygen to mediate cell death sometime after the irradiation event. The depletion of glutamine will present glutathione generation and a means of reactive oxygen mitigation. Further, glutamine can promote BRD4 activity to induce the expression of the oncogene c-myc. C-myc expression is a known means of irradiation resistance. While Bromo domain inhibitors (block BRD4 activity) may be used, antagonizing macropinocytosis, glutamine uptake, or glutaminase activity can sensitize a cancer type (e.g., breast, prostate, head & neck cancer) to radiation therapy.

In various embodiments, the present disclosure provides that glutamine detection in the blood may be used as a biomarker for to distinguish prostate cancer patients that have indolent and aggressive disease. In various embodiments, the present disclosure provides that blood glutamine may be used to distinguish pancreatitis patients that progress to pancreatic cancer. In various embodiments, the present disclosure provides that elevated glutamine in the blood would support further intervention as it would indicate aggressive prostate cancer or pancreatic cancer.

The inventors discovered that high fat diet leads to liver and pancreatic fibrosis in a glutamine dependent manner. Fatty liver and liver fibrosis (i.e. steatosis) is a pervasive disease that is growing with a number of men and women with issues of obesity, diabetes, and excessive alcohol use. The inventors discovered, high fat diet, long before evidence of fatty liver disease development, induces glutamine synthetase expression in a CD105 (endoglin) dependent manner. In various embodiments, the present disclosure provides that antagonizing glutamine uptake or glutaminase activity can reduce and potentially prevent the activation of liver and pancreatic stellate cells that are responsible for the expression of extracellular matrix proteins (e.g., collagen I, hyaluronic acid) and inflammatory cytokines (for the recruitment of immune cells).

### Androgen Deprivation Therapy Agents Used in the Methods and Compositions of the Present Invention

Androgen deprivation therapy (ADT), also called androgen suppression therapy, may include an antihormone therapy. In various embodiments, androgen deprivation therapy may be used to treat prostate cancer. In another embodiment, androgen deprivation therapy may be used to treat late stage bladder cancer and triple negative breast cancer too. The glutamine signaling is similarly active in bladder and breast cancer.

Non-limiting examples of androgen deprivation therapy agents may include at least one antiandrogen. In another embodiment, the androgen deprivation therapy agents may include at least one of: enzalutamide, bicalutamide, leuprolide, apalutamide, abiraterone, goserelin, triptorelin, and any combinations thereof.

As used herein, the term “antiandrogen” (also interchangeably called as androgen signaling inhibitor or blocker) refers to any agent that inhibits the androgen signaling, including inhibition of any molecular signaling steps upstream or downstream of androgen. An antiandrogen may be a small molecule; a nucleic acid such as siRNA, shRNA, and miRNA; a nucleic acid analogue such as PNA, pc-PNA, and LNA; an aptamer; a ribozyme; a peptide; a protein; an avimer; or an antibody, or variants and fragments thereof. Antiandrogens prevent androgens from expressing their biological effects on responsive cells, tissues and organs. Antiandrogens alter the androgen pathway by inhibiting androgen receptors (ARs) or suppressing androgen production. Examples of antiandrogens include but are not limited to AR ligands (androgen receptor ligands) such as AR antagonists (androgen receptor antagonists), AR inhibitors (androgen receptor inhibitors), and selective AR modulators (SARMs), and androgen synthesis inhibitors such as enzyme inhibitors and anti gonadotropins. Examples of AR antagonists include but are not limited to flutamide, nilutamide, bicalutamide, enzalutamide, apalutamide, cyproterone acetate, megestrol acetate, chlormadinone acetate, spironolactone, canrenone, drospirenone, ketoconazole, topilutamide (fluridil), and cimetidine. Examples of SARMs include but are not limited to andarine and enobosarm (ostarine). Examples of enzyme inhibitors include but are not limited to 5α-reductase inhibitors (e.g., finasteride, dutasteride, alfatradiol, and saw palmetto extract), CYP17A1 (e.g., 17α-hydroxylase/17,20-lyase) inhibitors (e.g., cyproterone acetate, spironolactone, danazol, gestrinone, ketoconazole, and abiraterone acetate), 3β-Hydroxysteroid dehydrogenase inhibitors (e.g., danazol, gestrinone, and abiraterone acetate), 17β-Hydroxysteroid dehydrogenase inhibitors (e.g., danazol and simvastatin), CYP11A1 (cholesterol side-chain cleavage enzyme) inhibitors (e.g., aminoglutethimide and danazol), and HMG-CoA reductase inhibitors (e.g., statins such as atorvastatin, simvastatin). Examples of antigonadotropins include but are not limited to progestogens (e.g., progesterone, cyproterone acetate, medroxyprogesterone acetate, megestrol acetate, chlormadinone acetate, spironolactone, and drospirenone), estrogens (e.g., estradiol, ethinyl estradiol, diethylstilbestrol, and conjugated equine estrogens), GnRH analogues such as GnRH agonists (e.g., buserelin, deslorelin, gonadorelin, goserelin, histrelin, leuprorelin, nafarelin, and triptorelin) and GnRH antagonists (e.g., abarelix, cetrorelix, degarelix, and ganirelix), and anabolic steroids (e.g., nandrolone and oxandrolone).

In various embodiments, the antiandrogen is flutamide, nilutamide, bicalutamide, enzalutamide, or apalutamide, or any of their functional equivalents, analogs, derivatives or salts. In some embodiments, the antiandrogen is enzalutamide, a functional equivalent, analog, derivative or salt of enzalutamide, or a combination thereof.

### Inhibitors Used in the Methods and Compositions of the Present Invention

As used herein, the term “inhibitor” means refers to any agent, substance, chemical or compound that inhibits, blocks, stops, or slows something from happening.

Non-limiting examples of inhibitors include glutamine metabolism inhibitors, glutaminase inhibitors, and macropinocytosis inhibitors. In some embodiments, at least one inhibitor is selected from the group comprising: at least one glutamine metabolism inhibitor, at least one glutaminase inhibitor, at least one macropinocytosis inhibitor, and combinations thereof.

As used herein, the term “glutamine metabolism inhibitor” (also interchangeably called glutamine uptake blocker or glutamine conversion blocker) refers to any agent, substance, chemical, or compound that inhibits the metabolism, uptake, and/or conversion of glutamine. Non-limiting examples of glutamine metabolism inhibitors include L-gamma-glutamyl-p-nitroanilide (GPNA), CAS #: 7300-59-6 or any of its functional equivalents, analogs, derivatives or salts. The glutamine metabolism inhibitor also includes direct glutaminase inhibitors, such as, for example, bis-2-(5-phenylacetamido-1,2,4-thiadiazol-2-yl)ethyl sulfide (BPTES) and CB-839. There are also other ways of blocking glutamine metabolism by blocking its cell uptake, blocking SCL1A5-gamma-1-Glutamyl-p-Nitroanilide (GPNA).

As used herein, the term “glutaminase inhibitor” refers to any agent, substance, chemical, or compound that inhibits glutaminase and its ability to convert glutamine to glutamate. Non-limiting examples of glutaminase inhibitors include bis-2(5-phenylacetamido-1,3,4-thiadiazol-2-yl)ethyl sulfide (BPTES), CAS #: 314045-39-1 or any of its functional equivalents, analogs, derivatives or salts; and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839) (CAS #: 1439399-58-2) or any of its functional equivalents, analogs, derivatives or salts.

As used herein, the term “macropinocytosis inhibitor” refers to any agent or compound that inhibits micropinocytosis, such as, 5-(N-ethyl-N-isopropyl) amiloride (EIPA).

As used herein, the term “CD105 inhibitor” refers to any agent, substance, chemical, or compound that inhibits CD105. In various embodiments, the CD105 inhibitor is an antibody specifically binding to CD105 or an antigen-binding fragment thereof. In various other embodiments, the CD105 antagonist is TRC105 or an antigen-binding fragment thereof.

Examples of anti-PD1 inhibitors include, but are not limited to pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, PF-06801591, anti-PD1 antibody expressing pluripotent killer T lymphocytes (PIK-PD-1), and autologous anti-EGFRvIII 4SCAR-IgT cells, and any applicable analogs, derivatives or salts thereof.

Examples of anti-PDL1 inhibitors include, but are not limited to BGB-A333, CK-301, FAZ053, KN035, MDX-1105, MSB2311, SHR-1316, atezolizumab, avelumab, durvalumab, BMS-936559, CK-301, and M7824, and any applicable analogs, derivatives or salts thereof.

Examples of anti-CD47 inhibitors include, but are not limited to anti-CD47 monoclonal antibody AO-176, anti-CD47 monoclonal antibody CC-90002, anti-CD47 monoclonal antibody Hu5F9-G4, anti-CD47 monoclonal antibody IBI188, anti-CD47 monoclonal antibody SHR-1603, anti-CD47 monoclonal antibody SRF231, CD47 antagonist ALX-148, and chimeric humanized anti-CD47 antibody, and any applicable analogs, derivatives or salts thereof.

### Dosages

Typical dosages of a therapeutically effective amount of an androgen deprivation therapy agent or an inhibitor disclosed herein may be in the ranges recommended by the manufacturer where known therapeutic molecules or compounds are used, and also as indicated to the skilled artisan by the in vitro responses in cells or in vivo responses in animal models. Such dosages typically may be reduced by up to about an order of magnitude in concentration or amount without losing relevant biological activity. The actual dosage may depend upon the judgment of the physician, the condition of the patient, and the effectiveness of the therapeutic method based, for example, on the in vitro responsiveness of relevant cultured cells or histocultured tissue sample, or the responses observed in the appropriate animal models. In various embodiments, the androgen deprivation therapy agent may be administered once a day (SID/QD), twice a day (BID), three times a day (TID), four times a day (QID), or more, so as to administer an effective amount of the androgen deprivation therapy agent to the subject, where the effective amount is any one or more of the doses described herein.

In various embodiments, the androgen deprivation therapy agent may be administered at about 0.001-1000 mg/m², greater than 1000 mg/m², or less than 0.001 mg/m². In various embodiments, the androgen deprivation therapy agent may be administered at about 0.001-1000 mg/kg, greater than 1000 mg/kg, or less than 0.001 mg/kg. In various embodiments, the androgen deprivation therapy agent may be administered once or more in the subject. In various embodiments, the androgen deprivation therapy agent may be administered about 1 or more times per year. In various embodiments, the androgen deprivation therapy agent is administered for about 1 day or more. Here, “mg/kg” refers to mg per kg body weight of the subject, and “mg/m²” refers to mg per m² body surface area of the subject. In certain embodiments, the androgen deprivation therapy agent is administered to a human.

In one embodiment, the androgen deprivation therapy agent may be administered at following dosage: Enzalutamide (40-80 mg per day), bicalutamide (50-150 mg per day), leuprolide (7.5 mg per month), apalutamide (60-240 mg per day), abiraterone (250 or 500 mg per day), goserelin (3.6 mg per month), and triptorelin (11.25 mg per 3 month).

In various embodiments, the effective amount of the androgen deprivation therapy agent may include any one or more of about 0.001-1000 μg/kg/day, greater than 1000 μg/kg/day, or less than 0.001 μg/kg/day. In various embodiments, the effective amount of the androgen deprivation therapy agent may include about 0.001 μg/m²/day, greater than 0.001 μg/m²/day, or less than 0.001 μg/m²/day. In various embodiments, the effective amount of the androgen deprivation therapy agent is any one or more of about 0.001-1000 mg/kg/day, greater than 1000 mg/kg/day, or less than 0.001 mg/kg/day. In various embodiments, the effective amount of the androgen deprivation therapy agent may include any one or more of about 0.001-1000 mg/m²/day, greater than 1000 mg/m²/day, or less than 0.001 mg/m²/day.

In some embodiments, the effective amount may include a therapeutically effective amount.

In some embodiments, the androgen deprivation therapy agent may be administered at the prevention stage of a condition (i.e., when the subject has not developed the condition but is likely to or in the process to develop the condition). In other embodiments, the androgen deprivation therapy agent may be administered at the treatment stage of a condition (i.e., when the subject has already developed the condition).

In accordance with the present disclosure, the androgen deprivation therapy agent may be administered using the appropriate modes of administration, for instance, the modes of administration recommended by the manufacturer for each of the androgen deprivation therapy agent. In accordance with the present disclosure, various routes may be utilized to administer the androgen deprivation therapy agent of the claimed methods, including but not limited to intravascular, intravenous, intraarterial, intratumoral, intramuscular, subcutaneous, intraperitoneal, intranasal, or oral.

In various embodiments, the inhibitor is administered at about 0.001-1000 mg/m², greater than 1000 mg/m², or less than 0.001 mg/m². In various embodiments, the inhibitor is administered at about 0.001-1000 mg/kg, greater than 1000 mg/kg, or less than 1000 mg/kg. In various embodiments, the inhibitor is administered once, twice, three or more times. In various embodiments, the inhibitor is administered about once or more a day. In various embodiments, the inhibitor is administered for about 1 day or more.

In various embodiments, the effective amount of the inhibitor may include about 0.001-1000 μg/kg/day, greater than 1000 μg/kg/day, or less than 0.001 μg/kg/day. In various embodiments, the effective amount of the androgen deprivation therapy agent may include about 0.001 μg/m²/day, greater than 0.001 μg/m²/day, or less than 0.001 μg/m²/day. In various embodiments, the effective amount of the androgen deprivation therapy agent is any one or more of about 0.001-1000 mg/kg/day, greater than 1000 mg/kg/day, or less than 0.001 mg/kg/day. In various embodiments, the effective amount of the androgen deprivation therapy agent may include any one or more of about 0.001-1000 mg/m²/day, greater than 1000 mg/m²/day, or less than 0.001 mg/m²/day.

In some embodiments, the inhibitor may be administered at the prevention stage of a condition (i.e., when the subject has not developed the condition but is likely to or in the process to develop the condition). In other embodiments, the inhibitor may be administered at the treatment stage of a condition (i.e., when the subject has already developed the condition).

In accordance with the present disclosure, the inhibitor may be administered using the appropriate modes of administration, for instance, the modes of administration recommended by the manufacturer for each of the inhibitor. In accordance with the present disclosure, various routes may be utilized to administer the inhibitor of the claimed methods, including but not limited to intravascular, intravenous, intraarterial, intratumoral, intramuscular, subcutaneous, intraperitoneal, intranasal, or oral.

### Methods of Treatment

In various embodiments, the present disclosure provides a method of treating, preventing, reducing the likelihood of having, reducing the severity of and/or slowing the progression of a disease or condition in a subject. The method may comprise or may consist of providing at least one macropinocytosis inhibitor, at least one glutamine metabolism inhibitor, at least one glutaminase inhibitor, and/or combinations thereof described herein and administering a therapeutically effective amount of the at least one macropinocytosis inhibitor, at least one glutamine metabolism inhibitor, at least one glutaminase inhibitor, and/or combinations thereof to the subject, thereby treating, preventing, reducing the likelihood of having, reducing the severity of and/or slowing the progression of the disease or condition in the subject.

In various embodiments, the present disclosure provides a method of treating, reducing the severity of and/or slowing the progression of a condition or disease in a subject, including: administering a therapeutically effective amount of at least one inhibitor to the subject, wherein the at least one inhibitor is selected from the group comprising: at least one macropinocytosis inhibitor, at least one glutamine metabolism inhibitor, at least one glutaminase inhibitor, and combinations thereof, thereby treating, reducing the severity of and/or slowing the progression of the condition or disease in the subject. In some embodiments, the method further comprises administering at least one androgen deprivation therapy to the subject.

In various embodiments, the present disclosure provides a method of treating, reducing the severity of and/or slowing the progression of and/or inhibiting the progression of a condition or disease in a subject, including: administering at least one androgen deprivation therapy to the subject; and administering at least one inhibitor to the subject, wherein the at least one inhibitor is selected from the group comprising: at least one macropinocytosis inhibitor, at least one glutamine metabolism inhibitor, at least one glutaminase inhibitor, and combinations thereof, thereby treating, reducing the severity of and/or slowing the progression of the condition or disease in the subject.

In some embodiments, the at least one androgen deprivation therapy comprises at least one therapy selected from at least one surgical technique, at least one androgen deprivation therapy agent, and any combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

### Methods for Treating Cancer

In various embodiments, the present disclosure provides a method of treating, reducing the severity of and/or slowing the progression of and/or inhibiting the progression of a cancer in a subject, including: administering at least one androgen deprivation therapy to the subject; and administering at least one inhibitor to the subject, wherein the at least one inhibitor is selected from the group consisting of: at least one macropinocytosis inhibitor, at least one glutamine metabolism inhibitor, at least one glutaminase inhibitor, and combinations thereof, thereby treating, reducing the severity of and/or slowing the progression of the cancer in the subject.

In various embodiments, the present disclosure provides a method of treating, reducing the severity of and/or slowing the progression of a cancer in a subject, including: administering an effective amount of at least one androgen deprivation therapy agent to the subject; and administering an effective amount at least one inhibitor to the subject, wherein the at least one inhibitor is selected from the group consisting of: at least one macropinocytosis inhibitor, at least one glutamine metabolism inhibitor, at least one glutaminase inhibitor, and combinations thereof, thereby treating, reducing the severity of and/or slowing the progression of the cancer in the subject.

In some embodiments, the at least one androgen deprivation therapy includes at least one selected from at least one surgical technique, at least one androgen deprivation therapy agent, and combinations thereof. In some embodiments, an effective amount is a therapeutically effective amount. In some embodiments, the cancer is prostate cancer.

In various embodiments, the method further comprises administering a CD105 inhibitor to the subject. In various embodiments, the method further comprises administering a CD105 inhibitor and an immune checkpoint inhibitor to the subject. In some embodiments, the immune checkpoint inhibitor is an anti-PD1 inhibitor, anti-PDL1 inhibitor, or anti-CD47 inhibitor, or combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

### Methods for Inhibiting Growth of a Cancer

A method of inhibiting growth of a cancer in a subject, including: administering at least one androgen deprivation therapy (or androgen deprivation therapy agent) to the subject; and administering an effective amount of at least one inhibitor to the subject, wherein the at least one inhibitor is selected from the group consisting of at least one macropinocytosis inhibitor, at least one glutamine metabolism inhibitor, at least one glutaminase inhibitor, and combinations thereof.

In some embodiments, the at least one androgen deprivation therapy may include at least one selected from at least one surgical technique, at least one androgen deprivation therapy agent, and combinations thereof. In some embodiments, an effective amount may include a therapeutically effective amount.

In various embodiments, the method further comprises administering a CD105 inhibitor to the subject. In various embodiments, the method further comprises administering a CD105 inhibitor and an immune checkpoint inhibitor to the subject. In some embodiments, the immune checkpoint inhibitor is an anti-PD1 inhibitor, anti-PDL1 inhibitor, or anti-CD47 inhibitor, or combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

### Methods for Treating Hormone Therapy Resistant Cancer

In various embodiments, the present disclosure provides a method of treating, reducing the severity of and/or slowing the progression of and/or inhibiting the progression of a hormone therapy resistant cancer in a subject, including: administering at least one inhibitor to the subject, wherein the at least one inhibitor is selected from the group consisting of: at least one macropinocytosis inhibitor, at least one glutamine metabolism inhibitor, at least one glutaminase inhibitor, and combinations thereof, thereby treating, reducing the severity of and/or slowing the progression of the hormone therapy resistant cancer in the subject.

In some embodiments, the subject has a cancer and is receiving a cancer treatment. In some embodiments, the cancer treatment includes at least one androgen deprivation therapy.

In some embodiments the cancer is prostate cancer. In some embodiments, the hormone therapy resistant cancer is hormone therapy resistant prostate cancer.

In various embodiments, the method further comprises administering a CD105 inhibitor to the subject. In various embodiments, the method further comprises administering a CD105 inhibitor and an immune checkpoint inhibitor to the subject. In some embodiments, the immune checkpoint inhibitor is an anti-PD1 inhibitor, anti-PDL1 inhibitor, or anti-CD47 inhibitor, or combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

### Methods for Treating Depression

A method for treating depression in a subject, including: administering at least one inhibitor selected from the group consisting of: at least one macropinocytosis inhibitor, at least one glutamine metabolism inhibitor, at least one glutaminase inhibitor, and combinations thereof to the subject, wherein the subject has a cancer and is receiving a cancer treatment, wherein the cancer treatment includes at least one androgen deprivation therapy.

In some embodiments, the androgen deprivation therapy includes administering a therapeutically effective amount of at least one androgen deprivation therapy agent.

In some embodiments, the depression may include cancer associated depression or a cancer therapy associated depression.

In various embodiments, the method further comprises administering a CD105 inhibitor to the subject. In various embodiments, the method further comprises administering a CD105 inhibitor and an immune checkpoint inhibitor to the subject. In some embodiments, the immune checkpoint inhibitor is an anti-PD1 inhibitor, anti-PDL1 inhibitor, or anti-CD47 inhibitor, or combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

### Methods for Sensitizing a Cancer to a Cancer Therapy

A method for sensitizing a cancer in a subject to at least one cancer therapy, including: administering an effective amount of at least one inhibitor to the subject, wherein the at least one inhibitor is selected from the group consisting of: at least one macropinocytosis inhibitor, at least one glutamine metabolism inhibitor, at least one glutaminase inhibitor, and any combinations thereof.

In some embodiments, the at least one cancer therapy is at least one selected from the group comprising: androgen deprivation therapy, chemotherapy, radiation therapy, and combinations thereof.

In various embodiments, the method further comprises administering a CD105 inhibitor to the subject. In various embodiments, the method further comprises administering a CD105 inhibitor and an immune checkpoint inhibitor to the subject. In some embodiments, the immune checkpoint inhibitor is an anti-PD1 inhibitor, anti-PDL1 inhibitor, or anti-CD47 inhibitor, or combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

Methods for Preventing and/or Reducing Activation of Stellate Cells

A method for preventing and/or reducing activation of stellate cells in a subject, the method including: administering an effective amount of at least one inhibitor to the subject, wherein the at least one inhibitor is selected from the group consisting of: at least one macropinocytosis inhibitor, at least one glutamine metabolism inhibitor, at least one glutaminase inhibitor, and combinations thereof.

In some embodiments, the stellate cells are selected from the group comprising: hepatic stellate cells, pancreatic stellate cells, and any combinations thereof.

In some embodiments, the method further includes administering at least one chemotherapeutic agent. Non-limiting examples of the chemotherapeutic agent include Temozolomide, Actinomycin, Alitretinoin, All-trans retinoic acid, Azacitidine, Azathioprine, Bevacizumab, Bexatotene, Bleomycin, Bortezomib, Carboplatin, Capecitabine, Cetuximab, Cisplatin, Chlorambucil, Cyclophosphamide, Cytarabine, Daunorubicin, Docetaxel, Doxifluridine, Doxorubicin, liposome-encapsulated Doxorubicin such as Doxil (pegylated form), Myocet (nonpegylated form) and Caelyx, Epirubicin, Epothilone, Erlotinib, Etoposide, Fluorouracil, Gefitinib, Gemcitabine, Hydroxyurea, Idarubicin, Imatinib, Ipilimumab, Irinotecan, Nanoliposomal Irinotecan (Nal-IRI), Mechlorethamine, Melphalan, Mercaptopurine, Methotrexate, Mitoxantrone, Ocrelizumab, Ofatumumab, Oxaliplatin, Paclitaxel, Protein-Bound Paclitaxel, Nab-Paclitaxel, Panitumab, Pemetrexed, Rituximab, Tafluposide, Teniposide, Tioguanine, Topotecan, Tretinoin, Valrubicin, Vemurafenib, Vinblastine, Vincristine, Vindesine, Vinorelbine, Vorinostat, Romidepsin, 5-fluorouracil (5-FU), 6-mercaptopurine (6-MP), Cladribine, Clofarabine, Floxuridine, Fludarabine, Pentostatin, Mitomycin, ixabepilone, Estramustine, prednisone, methylprednisolone, dexamethasone or a combination thereof.

In various embodiments, the chemotherapeutic agent is a platinum-based antineoplastic agent. Examples of the platinum-based antineoplastic agent include but are not limited to oxaliplatin, cisplatin, lipoplatin (a liposomal version of cisplatin), carboplatin, satraplatin, picoplatin, nedaplatin, and triplatin.

In various embodiments, the chemotherapeutic agent may include a taxane. Examples of the taxane may include, but are not limited to, paclitaxel, docetaxel, and cabazitaxel. In certain embodiments, the chemotherapeutic agent may include paclitaxel, or its functional equivalent, analog, derivative, variant or salt, or any combinations thereof. In some embodiments, the chemotherapeutic agent may include protein-bound paclitaxel or nab-paclitaxel.

In various embodiments, the chemotherapeutic agent may include an anthracycline. Examples of the anthracycline may include, but are not limited to, doxorubicin, daunorubicin, epirubicin, idarubicin, pirarubicin, aclarubicin, valrubicin, and mitoxantrone. In certain embodiments, the chemotherapeutic agent may include doxorubicin, or its functional equivalent, analog, derivative, variant or salt, or a combination thereof.

In some embodiments, administration of the at least one inhibitor is before, after, sequentially, or simultaneous to administration of the androgen deprivation therapy and/or androgen deprivation therapy agent.

In various embodiments, the method further comprises administering a CD105 inhibitor to the subject. In various embodiments, the method further comprises administering a CD105 inhibitor and an immune checkpoint inhibitor to the subject. In some embodiments, the immune checkpoint inhibitor is an anti-PD1 inhibitor, anti-PDL1 inhibitor, or anti-CD47 inhibitor, or combinations thereof.

In various embodiments, the subject has been determined to have a level or amount of glutamine, glutamine metabolite, or both that is elevated compared to reference levels or amounts of glutamine, glutamine metabolite, or both. The reference amount or level can be from a reference sample obtained from a control subject (or an average from a number of control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

### Combination Therapies

In some embodiments, the methods may optionally further include before, after, sequentially, or simultaneously providing and/or administering additional therapies including by not limited to chemotherapy, radiation, radiation therapy, or a combination thereof. In some embodiments, the chemotherapy includes at least one chemotherapeutic agent.

Methods for Diagnosing, Detecting, and/or Identifying a Condition

A method for diagnosing, detecting, and/or identifying a condition or disease in a subject, the method including: measuring and/or quantifying and/or determining an amount or level of an analyte in the subject, wherein a change in the amount or level of the analyte in the subject relative to an amount or level of the analyte in a reference sample is indicative of a condition or disease in the subject, wherein the analyte is selected from the group comprising: glutamine, a glutamine metabolite, and any combinations thereof. The analyte may include glutamine and/or a glutamine metabolite. The disease or condition may include a cancer, a tumor, or any other diseases.

In some embodiments, the measuring, quantifying, and/or determining may be performed using at least one selected from the group including: spectral magnetic resonance imaging, sample assay, and any combinations thereof.

In some embodiments, the sample assay may include a body fluid assay. In some embodiments, the sample assay may be a blood assay. In some embodiments, no radioactive tracer or contrast agent may be administered to the subject. In some embodiments, the reference sample may be obtained from a control subject (or an average from control subjects (e.g., 1-100, 1-250, 1-500, 1-1000, or 10, 50, 100, 150, 200, 300, 400, 500, 750, 1000, etc.), wherein the control subject does not have the condition or disease (e.g., cancer, androgen deprivation therapy resistance, or the like).

In some embodiments, the reference sample may be obtained from the subject before the subject is treated for the condition or disease. In some embodiments, the reference sample may be from a subject that has been successfully treated for the condition or disease. In some embodiments, the condition may include androgen deprivation therapy resistance. In some embodiments, the condition may include a likelihood or risk of developing a disease, such as, for example, cancer.

### Methods for Detecting Hormone Therapy Resistant Cancer

A method for detecting a hormone therapy resistant cancer in a subject, including: measuring and/or quantifying and/or determining an amount or level of an analyte in the subject; comparing the amount or level of the analyte in the subject to an amount or level of the analyte in a reference sample, wherein a change in the amount or level of the analyte in the subject relative to an amount or level of the analyte in a reference sample is used to detect the hormone therapy resistant cancer in the subject, wherein the analyte is selected from the group including: glutamine, a glutamine metabolite, and any combinations thereof.

In some embodiments, the subject has a cancer and is receiving a cancer treatment. In some embodiments, the cancer treatment includes at least one androgen deprivation therapy.

In some embodiments the cancer is prostate cancer. In some embodiments, the hormone therapy resistant cancer is hormone therapy resistant prostate cancer.

### Methods for Prognosing Hormone Therapy Resistant Cancer

A method for predicting the development of and/or prognosing a hormone therapy resistant cancer in a subject, including: measuring and/or quantifying and/or determining an amount or level of an analyte in the subject; comparing the amount or level of the analyte in the subject to an amount or level of the analyte in a reference sample, wherein a change in the amount or level of the analyte in the subject relative to an amount or level of the analyte in a reference sample is used to detect the hormone therapy resistant cancer in the subject, wherein the analyte is selected from the group comprising: glutamine, a glutamine metabolite, and combinations thereof.

In some embodiments, the subject has a cancer and is receiving a cancer treatment. In some embodiments, the cancer treatment includes at least one androgen deprivation therapy.

In some embodiments the cancer is prostate cancer. In some embodiments, the hormone therapy resistant cancer is hormone therapy resistant prostate cancer.

Methods for Detecting a Cancer Distinguishably from a Non-Cancer Condition

A method for detecting a cancer distinguishably from a non-cancer condition in a subject, including: measuring and/or quantifying and/or determining an amount or level of glutamine and/or a glutamine metabolite in the subject; comparing the amount or level of glutamine and/or the glutamine metabolite in the subject to an amount or level of glutamine and/or the glutamine metabolite in a reference sample, wherein a change in the amount or level of glutamine and/or the glutamine metabolite in the subject relative to an amount or level of glutamine and/or the glutamine metabolite in a reference sample is used to distinguish the cancer from the non-cancer condition in the subject.

In some embodiments, the change in the amount or level of glutamine and/or a glutamine metabolite in the subject may include an increase in the amount or level of glutamine and/or the glutamine metabolite in the subject relative to the amount or level of glutamine and/or the glutamine metabolite in the reference sample.

In some embodiments, the change in the amount or level of glutamine and/or a glutamine metabolite in the subject is a decrease in the amount or level of glutamine and/or the glutamine metabolite in the subject relative to the amount or level of glutamine and/or the glutamine metabolite in the reference sample.

In some embodiments, the cancer is pancreatic cancer. In some embodiments, the non-cancer condition is pancreatitis. In some embodiments, the cancer is breast cancer. In some embodiments, the non-cancer condition is ductal carcinoma in situ. In some embodiments, the cancer is lung cancer. In some embodiments, the non-cancer condition is lung fibrosis. As Ras oncogene activation is the determinant factor for the detection of elevated glutamine levels, the detectable cancers are not limited to those listed.

Methods for Methods for Detecting a Cancer Distinguishably from a Non-Cancer Condition

A method for detecting a first cancer type distinguishably from a second cancer type in a subject, including: measuring and/or quantifying and/or determining an amount or level of an analyte in the subject; comparing the amount or level of the analyte in the subject to an amount or level of the analyte in a reference sample, wherein a change in the amount or level of the analyte in the subject relative to an amount or level of the analyte in a reference sample is used to distinguish the first cancer type from the second cancer type in the subject, wherein the analyte is selected from the group comprising: glutamine, a glutamine metabolite, and any combinations thereof.

In some embodiments, the first cancer type may include indolent cancer, indolent prostate cancer, or an aggressive cancer (e.g., aggressive prostate cancer). In some embodiments, the second cancer type may include indolent cancer, indolent prostate cancer, or an aggressive cancer.

Markers for Detecting, Diagnosing, and/or Identifying a Cancer

In various embodiments, the present disclosure provides a marker for detecting a cancer in a subject, including at least one selected from the group including: glutamine, glutamine metabolite, and any combinations thereof.

In some embodiments, the glutamine metabolite may be selected from the group including: glutamate, glutathione, γ-aminobutyrate, α-ketoglutarate, succinate, fumarate, malate, acetyl-CoA, 2-hydroxyglutarate and any combinations thereof.

Markers for Detecting, Diagnosing, and/or Identifying a Condition or Disease

In various embodiments, the present disclosure provides a marker for detecting a condition or disease in a subject, including at least one selected from the group including: glutamine, glutamine metabolite, and any combinations thereof.

In some embodiments, the glutamine metabolite is selected from the group including: glutamate, glutathione, γ-aminobutyrate, α-ketoglutarate, succinate, fumarate, malate, acetyl-CoA, 2-hydroxyglutarate and any combinations thereof.

### Personalized Treatments

Various embodiments provide for a method of treating, reducing the severity of, slowing the progression of, or inhibiting the progression of a cancer, cancer metastasis, tumor or tumor metastasis, or sensitizing a cancer, cancer metastasis, tumor or tumor metastasis to a cancer therapy in a subject, comprising obtaining or requesting the results of an analysis of glutamine, glutamine metabolite or both, and administering a glutaminase inhibitor, a glutamine metabolism inhibitor, a micropinocytosis inhibitor or a combination thereof to the subject when the analysis of glutamine, glutamine metabolite or both indicates that glutamine, glutamine metabolite or both are elevated.

In various embodiments, the method further comprises administering an androgen deprivation therapy to the subject; for example, if the subject has prostate cancer. In various embodiments, the androgen deprivation therapy comprises surgical castration or an antiandrogen.

In various embodiments, the method further comprises administering a cancer therapy to the subject if the subject has cancer.

In various embodiments, the cancer is selected from the group consisting of prostate cancer, pancreatic cancer, breast cancer, lung cancer, colon cancer, liver cancer, and combinations thereof.

In various embodiments, the cancer is a Ras mutated cancer. In various embodiments, the cancer is an adenocarcinoma or a neuroendocrine cancer.

In various embodiments, the method further comprises administering a CD105 inhibitor to the subject.

In various embodiments, the method further comprises administering a CD105 inhibitor and an immune checkpoint inhibitor to the subject. In various embodiments, the immune checkpoint inhibitor is an anti-PD1 inhibitor, anti-PDL1 inhibitor, or anti-CD47 inhibitor.

Various embodiments of the invention provide for a method of treating, reducing the severity of, slowing the progression of, or inhibiting the progression of a disease or condition, or preventing or reducing activation of stellate cells, in a subject in need thereof, comprising: obtaining or requesting the results of an analysis of glutamine, glutamine metabolite or both, and administering glutaminase inhibitor, a glutamine metabolism inhibitor, a micropinocytosis inhibitor or a combination thereof to the subject. In various embodiments, the condition is selected from the group consisting of liver fibrosis, pancreatic fibrosis, cancer associated depression and combinations thereof.

In various embodiments, the analysis of glutamine, glutamine metabolite or both comprises detecting the glutamine, glutamine metabolite, or both; quantifying the amount of glutamine, glutamine metabolite, or both; comparing the amount of the glutamine to a reference amount of glutamine, comparing the amount of the glutamine metabolite to a reference amount of glutamine metabolite, or both; and determining whether the amount glutamine, a glutamine metabolite or both is elevated compared to the reference amount of glutamine, the reference amount glutamine metabolite, or both, in the subject in need thereof.

### Compositions

A composition, including: at least one androgen deprivation therapy agent; and at least one inhibitor selected from the group comprising: at least one macropinocytosis inhibitor, at least one glutamine metabolism inhibitor, at least one glutaminase inhibitor, and combinations thereof.

A composition for treating, preventing, reducing the likelihood of having, reducing the severity of and/or slowing the progression of a cancer in a subject, wherein the composition includes: at least one androgen deprivation therapy agent; and at least one inhibitor selected from the group comprising: at least one macropinocytosis inhibitor, at least one glutamine metabolism inhibitor, at least one glutaminase inhibitor, and combinations thereof.

In some embodiments, at least one androgen deprivation therapy agent is selected and/or at least one androgen receptor signaling inhibitor.

Product Combinations

A product combination, including: at least one androgen deprivation therapy agent; and at least one inhibitor selected from the group comprising: at least one macropinocytosis inhibitor, at least one glutamine metabolism inhibitor, at least one glutaminase inhibitor, and combinations thereof.

A product combination for treating, preventing, reducing the likelihood of having, reducing the severity of and/or slowing the progression of a cancer in a subject, wherein the composition includes: at least one androgen deprivation therapy agent; and at least one inhibitor selected from the group comprising: at least one macropinocytosis inhibitor, at least one glutamine metabolism inhibitor, at least one glutaminase inhibitor, and combinations thereof.

A product combination for treating a cancer in a subject, wherein the composition includes: at least one androgen deprivation therapy agent; and at least one inhibitor selected from the group comprising: at least one macropinocytosis inhibitor, at least one glutamine metabolism inhibitor, at least one glutaminase inhibitor, and combinations thereof.

A product combination for inhibiting and/or delaying growth of a cancer in a subject, wherein the composition includes: at least one androgen deprivation therapy agent; and at least one inhibitor selected from the group comprising: at least one macropinocytosis inhibitor, at least one glutamine metabolism inhibitor, at least one glutaminase inhibitor, and combinations thereof.

### Pharmaceutical Compositions

In various embodiments, the inhibitors and/or androgen deprivation therapy agents and/or compositions may be provided as pharmaceutical compositions. In various embodiments, the pharmaceutical compositions according to the present disclosure may be formulated for delivery via any route of administration. “Route of administration” may refer to any administration pathway known in the art, including but not limited to aerosol, nasal, via inhalation, oral, transmucosal, transdermal, parenteral, enteral, topical or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the pharmaceutical compositions may be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the pharmaceutical compositions may be in the form of aerosol, lotion, cream, gel, ointment, suspensions, solutions or emulsions. Methods for these administrations are known to one skilled in the art. In certain embodiments, the pharmaceutical compositions are formulated for intravascular, intravenous, or intraarterial administration. In one embodiment, the pharmaceutical compositions are formulated for intravenous administration as a single bolus.

In various embodiments, the pharmaceutical compositions according to the present disclosure may contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the present disclosure may contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its imaging benefits.

The pharmaceutical compositions according to the present disclosure may also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical compositions are made following the conventional techniques of pharmacy involving dry milling, mixing, and blending for powder forms; milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the present disclosure may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins P A, USA) (2000).

Before administration to patients, formulants may be added to the pharmaceutical composition. In some embodiments, the formulant is a liquid formulant. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In some embodiments, the sugar or sugar alcohol concentration is between 1.0 w/v % and 7.0 w/v %. In some embodiments, the sugar or sugar alcohol concentration is between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

A buffer may also be used in the pharmaceutical compositions to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that may be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The pharmaceutical compositions of the present disclosure may be sterilized by conventional, well-known sterilization techniques. The resulting solutions may be packaged for use or filtered under aseptic techniques and lyophilized, the lyophilized preparation being combined with a sterile solution prior to administration. The pharmaceutical compositions may contain pharmaceutically-acceptable auxiliary substances as required to approximate physiological parameters, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

In various embodiments, the present disclosure provides a pharmaceutical composition, including: at least one inhibitor selected from the group comprising: at least one macropinocytosis inhibitor, at least one glutamine metabolism inhibitor, at least one glutaminase inhibitor, and combinations thereof.

In some embodiments, the pharmaceutical composition further includes at least one androgen deprivation therapy agent. In some embodiments, the at least one androgen deprivation therapy agent is at least one antiandrogen. In some embodiments, the at least one antiandrogen is at least one selected from the group comprising: AR antagonist, androgen synthesis inhibitor, antigonadotropin, and combinations thereof. In some embodiments, the at least one androgen deprivation therapy agent is at least one selected from androgen receptor signaling inhibitor, androgen receptor inhibitor, and combinations thereof.

In some embodiments, the glutamine metabolism inhibitor is selected from the group comprising: L-gamma-glutamyl-p-nitroanilide (GPNA) and analogs, homologs, derivatives, and salts thereof.

In some embodiments, the glutaminase inhibitor is selected from the group comprising: bis-2(5-phenylacetamido-1,3,4-thiadiazol-2-yl)ethyl sulfide (BPTES) and analogs, homologs, derivatives, and salts thereof, and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839) and analogs, homologs, derivatives, and salts thereof.

### Kits

In various embodiments, the present disclosure provides a kit for treating, reducing the severity of and/or slowing the progression of a disease or condition in a subject. In various embodiments, the kit includes: a quantity of at least one androgen deprivation therapy agent described herein; a quantity of at least one inhibitor selected from the group comprising: at least one macropinocytosis inhibitor, at least one glutamine metabolism inhibitor, at least one glutaminase inhibitor, and combinations thereof; instructions for using the androgen deprivation therapy agent; and instructions for using the inhibitor. In some embodiments, the kit may further include a chemotherapeutic agent, and instructions for using the chemotherapeutic agent.

In various embodiments, the kit includes: at least one inhibitor selected from the group comprising: at least one macropinocytosis inhibitor, at least one glutamine metabolism inhibitor, at least one glutaminase inhibitor, and combinations thereof; and instructions for using the kit and the inhibitor.

The kit may include an assemblage of materials or components, including at least one of the inventive compositions; and/or at least one androgen deprivation therapy agent, and at least one inhibitor selected from the group comprising: at least one macropinocytosis inhibitor, at least one glutamine metabolism inhibitor, at least one glutaminase inhibitor, and combinations thereof. The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit may be provided to the practitioner stored in any convenient and suitable ways that preserve their operability and utility. For example, the components may be in dissolved, dehydrated, or lyophilized form; they may be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package may be a glass vial used to contain suitable quantities of a composition as described herein. The packaging material generally has an external label which indicates the contents and/or purpose of the kit and/or its components.

TABLE 1
Association of patient androgen targeted therapy responsiveness and
plasma glutamine levels.

| | | | PSA | PSA on | | Glutamine |
|---|---|---|---|---|---|---|
| Patient | Rx | CRPC | baseline | treatment | Outcome | (μmol/L) |
| 1 | A | yes | 16.4 | 6.4 | Responsive | 64.6 |
| 2 | B + L | no | 52.2 | 0.75 | Responsive | 198.4 |
| 3 | L | no | 1.6 | 0.2 | Responsive | 337.5 |
| 4 | B | no | 21.3 | 11.3 | Responsive | 458.3 |
| 5 | L | no | 6.5 | <0.1 | Responsive | 539.8 |
| 6 | A + A5 | yes | 9.2 | 0.1 | Responsive | 870.4 |
| 7 | B + L | no | 16.2 | 2.3 | Responsive | 901.9 |
| 8 | L + T | no | 10.7 | 0.2 | Responsive | 901 |
| 9 | E | no | 0.2 | 0.1 | Responsive | 903 |
| 10 | B + L | no | 0.2 | 0.1 | Responsive | 906.9 |
| 11 | E | yes | 284.7 | 344.3 | Non- | 1035 |
| | | | | | Responsive | |
| 12 | A + A5 | yes | 63.5 | 116.5 | Non- | 1037.6 |
| | | | | | Responsive | |
| 13 | L | yes | 7.3 | 7.8 | Non- | 1305 |
| | | | | | Responsive | |
| 14 | E + R | yes | 51.9 | 71.5 | Non- | 1338 |
| | | | | | Responsive | |
| 15 | A + A5 | yes | 218 | 522.3 | Non- | 1359 |
| | | | | | Responsive | |
| 16 | E | yes | 0.8 | <0.1 | Responsive | 1650 |
| 17 | L | no | 14.3 | 0.1 | Responsive | 1809.5 |
| 18 | A | yes | 28.5 | 26.2 | Non- | 2030 |
| | | | | | Responsive | |
| 19 | A + A5 | yes | 4.2 | 0.8 | Responsive | 2090 |
| 20 | L | no | 5.3 | 1.1 | Responsive | 2394 |
| 21 | B + L + R | no | 5.7 | 0.1 | Responsive | 2535 |
| 22 | B + G | no | 17.9 | 5.7 | Non- | 2982 |
| | | | | | Responsive | |
| 23 | A + A5 | yes | 61.5 | 19.8 | Responsive | 3184.4 |
| 24 | A + R | yes | 17.7 | 25.6 | Non- | 3613.5 |
| | | | | | Responsive | |
| 25 | B + L | yes | 0.4 | 3.7 | Non- | 3751.1 |
| | | | | | Responsive | |
| 26 | L | yes | 0.9 | 1.2 | Non- | 3870 |
| | | | | | Responsive | |
| 27 | E | yes | 61.3 | 116.7 | Non- | 4369.8 |
| | | | | | Responsive | |
| 28 | B + L | yes | 4.4 | 0.8 | Non- | 4989 |
| | | | | | Responsive | |

Androgen targeted therapeutic treatment: A - Abiraterone, A5 - Apalutamide, B - Bicalutamide, E - Enzalutamide, G - Goserelin, L - Leuprolide, R - Radiation, T - Triptorelin

TABLE 2
Statistical analysis for FIG. 4E.
RAS CM Introduces NED P Values

| Gene ID | RAS vs. WT |
|---|---|
| AURKA | 0.301221545 |
| CHGA | 0.115214879 |
| CHGB | 0.042668993 |
| EN02 | 0.002282061 |
| FOXA2 | 0.000591958 |
| NKX2.2 | 0.011728483 |
| NGN3.3 | 0.000630702 |

TABLE 3
Statistical analysis for FIG. 4E
NED Expression P-Values

| | Glutamine | Glut+mTORi vs. |
|---|---|---|
| Gene ID | vs. Control | Control |
| CHA | 0.01 | 1.47E−06 |
| FOXA2 | 0.01 | 0.004050679 |
| SCG3 | 0.008 | 0.000159964 |
| ENO2 | 0.006 | 0.224168157 |
| NGN3 | 0.08 | 0.02504569 |
| MYCN | 0.05 | 0.259416618 |
| NKX2.2 | 0.01 | 0.004050679 |
| AURK A | 0.004 | 0.000273876 |

TABLE 4
Polymerase Chain Reaction Primers.
Primers for Real Time PCR

| | | SEQ ID |
|---|---|---|
| GENE | Sequence | NO: |
| RASAL3 (h) F: | CTC GTG GCA CAG GAT TAC CT | 1 |
| RASAL3 (h) R: | AGA CCT CGC AGC TGT T | 2 |
| Postn (m) F | TGC CCA GCA GTT TTG CCC AT | 3 |
| Postn (m) R | CGT TGC TCT CCA AAC CTC TA | 4 |
| Vim (m) F | CGG CTG CGA GAG AAA TTG C | 5 |
| Vim (m) R | CCA CTT TCC GTT CAA GGT CAA G | 6 |
| Fap (m) F | ATC TAT GAC CTT AGC AAT GGA GAA TTT G | 7 |
| Fap (m) R | GTT TTG ATA GAC ATA TGC TAA TTT ACT CCC AAC | 8 |
| Fsp1 (m) F | GGC AAG ACC CTT GGA G | 9 |
| Fsp1 (m) R | CCT TTT CCC CAG GAA GCT AG | 10 |
| Cox2 (m) F | CAG ACA TAA ACT GCG CCT T | 11 |
| Cox2 (m) R | GAT ACA CCT CTC CAC CAA TGA CC | 12 |
| Tnc (m) F | ACC ATG GGT ACA GGC TGT TG | 13 |
| Tnc (m) R | CCT TCT GCA CTG AAG TTG CC | 14 |
| Mmp-1 (m) F | CAC AAC AAT CCT CGT TGG AC | 15 |
| Mmp-1 (m) R | TGG TGT CAC ATC ACT CCA GA | 16 |
| Mmp-3 (m) F | CAG ACT TGT CCC GTT TCC AT | 17 |
| Mmp-3 (m) R | GGT GCT GAC TGC ATC AAA GA | 18 |
| SLC1A5 (h) F | CAA GGA GGT GCT CGA TTC GT | 19 |
| SLC1A5 (h) R | ACC CTG GTT CCG GTG ATA TTC | 20 |
| GLS (h) F | GGT CTC TGG ATA AGA TGG | 21 |
| GLS (h) R | CCC GTT GTC AGA ATC TCC TTG AGG | 22 |
| GLS2 (h) F | GGC AGA GAG ACG CCA CAC AG | 23 |
| GLS2 (h) R | AGT GGC CTT TAG TGC AGT GGT | 24 |
| SLC38A (h) F | GTG TTA ATG GCT GTG ACC CTG AC | 25 |
| SLC38A2 (h) R | GAG ACT ATG ACG CCA ACT GA | 26 |
| FOXA2 (h) F | GGCCAGAGTTCCACAAATCTA | 27 |
| FOXA2 (h) R | CCCTCCCTCCTTCTTGAAATAAT | 28 |
| NGN3 (h) F | CTGTGGGTGCTAAGGGTAAG | 29 |
| NGN3 (h) R | GGGAGAAGCAGAAGGAACAA | 30 |
| NKX2.2 (h) F | AAACCGTCCCAGCGTTAAT | 31 |
| NKX2.2 (h) R | CGGCTGACAATATCGCTACTC | 32 |
| ENO2 (h) F | AGTGGACCACATCAACTCCA | 33 |
| ENO2 (h) R | TGTCCAGTTTCTCTTGCTCCA | 34 |
| CHGB (h) F | TTCCATGCCAGTGGATAACA | 35 |
| CHGB (R) F | GAGAGGACCTCAATGATGCA | 36 |
| CHGA (h) F | CTCCCTGTGAACAGCCCTA | 37 |
| CHGA (h) R | TGTGTCGGAGATGACCTCAA | 38 |
| AURA (h) F | GTGTGCCTTAACCTCCCTATTC | 39 |
| AURA (h) R | CGAACCTTGCCTCCAGATTAT | 40 |
| COX2 (h) F | ATCATTCACCAGGCAAATTGC | 41 |
| COX2 (h) R | GGCTTCAGCATAAAGCGTTTG | 42 |
| MYCN (h) F | AGTACCTCCGGAGAGGACAC | 43 |
| MYCN (h) R | GGAACGCCGCTTCTCCA | 44 |
| SCG (h) F | CCAGGCAGTAAGGTAGAAATAGAG | 45 |
| SCG (h) R | GCAGGGACCTGAAGTTCATTA | 46 |
| β-actin F (h) | TCA CCC ACA CTG TGC CCA TCT ACG A | 47 |
| β-actin R (h) | CAG CGG AAC CGC TCA TTG CCA ATG G | 48 |
| GAPDH (h) F | CAT GAG AAG TAT GAC AAC AGC CT | 49 |
| GAPDH (h) R | AGT CCT TCC ACG ATA CCA AAG T | 50 |
| Gapdh (m) F | CAG CGA CAC CCA CTC CAC CTT | 51 |
| Gapdh (m) R | CAT GAG GTC CAC CCT GTT GCT | 52 |

Primers for Bisulphite Sequencing

| | | SEQ ID |
|---|---|---|
| GENE | Sequence | NO: |
| BS RASAL3 | TGA GGG TAG TAT GTG GAT AGA TAT TG | 53 |
| Ex2 Site I F | | |
| BS RASAL3 | ACT CCT AAT AAC TCA AAA CCC TAC C | 54 |
| Ex2 Site I R | | |
| BS RASAL3 | GGG TAG GGT TTT GAG TTA GGA G | 55 |
| Ex2 Site II F | | |
| BS RASAL3 | ATA AAT TCT TAT ACC TCC CCC AAA A | 56 |
| Ex2 Site II R | | |
| BS RASAL3 | TTT TGG GGG AGG TAT AAG AAT TTA T | 57 |
| Ex2 Site III F | | |
| BS RASAL3 | AAA ACA AAC AAA ACC CAA ACA TC | 58 |
| Ex2 Site III R | | |

Regular RT- polymerase chain reaction primers

| | | SEQ ID |
|---|---|---|
| Gene | Sequence | NO: |
| RASAL3 (h) F: | CTC GTG GCA CAG GAT TAC CT | 59 |
| RASAL3 (m) R: | AGA CCT CGC AGC TGT T | 60 |

Primers for MS-PCR

| | | SEQ ID |
|---|---|---|
| GENE | Sequence | NO: |
| RASAL3 M F | TTA TCG TTG GTA TAT AGG GGG C | 61 |
| RASAL3 M R | AAA CCT TAA AAA ATC GAA AAC GAC | 62 |
| RASAL3 U F | TTT ATT GTT GGT ATA TAG GGG GTG | 63 |
| RASAL3 U R | AAA ACC TTA AAA AAT CAA AAA CAA C | 64 |

H: human m: mouse

TABLE 5
Antibody Information.

| Name | Description | Cat No. | Company |
|---|---|---|---|
| RASAL3 | | 108033 | Abcam |
| αGLS1 | | 07-473 | Millipore |
| phospho-mTOR | Ser2448 | 2971S | Cell Signaling |
| | | | Technology |
| phospho-p44/42 | Thr202/Tyr204 | 4370 | Cell Signaling |
| MAPK (Erk1/2) | | | Technology |
| phospho-c-Raf | Ser289/296/301 | 9431 | Cell Signaling |
| | | | Technology |
| phospho-c-Raf | Ser259 | 9421 | Cell Signaling |
| | | | Technology |
| c-Raf | D5X6R | 12552 | Cell Signaling |
| | | | Technology |
| ARK-1 | | sc-56881 | Santa Cruze |
| FoxM1 | D-4 | 13147-1-AP | Proteintech |
| actin | | sc-47778 | Santa Cruze |
| SLC1A5 | | NBP1-89327 | Novus Biologicals |
| anti-chromogranin A | | sc-13090 | Santa Cruze |
| Anti-phosphorylated | | 06-570 | Millipore |
| histone H3 | | | |

TABLE 6
Reagent Information.

| Name | Cat No. | Company |
|---|---|---|
| 5-(N-Ethyl-N-isopropyl)amiloride | A3085 | Sigma |
| L-Glutamic acid γ-(p-nitroanilide) | G6133 | Sigma |
| hydrochloride (GPNA) | | |
| N,N′-[Thiobis(2,1-ethanediyl-1,3,4- | 5301 | Tocris |
| thiadiazole-5,2-diyl)]bisbenzeneacetamide | | |
| (BPTES) | | |
| Dextran, Tetramethylrhodamine, 70,000 | D-1818 | Life |
| MW, Lysine Fixable | | Technologies |
| LysoTracker ® Green DND-26 | L-7526 | Life |
| | | Technologies |
| DQ ™ Green BSA - Special Packaging | D12050 | Fisher |
| | | Scientific |
| TUNEL | S7100 | Millipore |
| h KRAS (G12V) (GP) inducible lentiviral | LVP799-GP | GenTarget |
| particles | | Inc |

Various embodiments of the present invention also include the following numbered paragraphs.
1. A method of treating, reducing the severity of and/or slowing the progression of prostate cancer in a subject, comprising: administering at least one androgen deprivation therapy to the subject; and administering a therapeutically effective amount of at least one inhibitor to the subject, wherein the at least one inhibitor is selected from the group comprising: at least one macropinocytosis inhibitor, at least one glutamine metabolism inhibitor, at least one glutaminase inhibitor, and any combinations thereof.
2. A composition for treating prostate cancer in a subject, wherein the composition comprises: at least one androgen deprivation therapy agent; and at least one inhibitor selected from the group comprising: at least one macropinocytosis inhibitor, at least one glutamine metabolism inhibitor, at least one glutaminase inhibitor, and any combinations thereof.
3. A composition for inhibiting and/or delaying growth of prostate cancer in a subject, wherein the composition comprises: at least one androgen deprivation therapy agent; and at least one inhibitor selected from the group comprising: at least one macropinocytosis inhibitor, at least one glutamine metabolism inhibitor, at least one glutaminase inhibitor, and any combinations thereof.
4. A method for detecting a condition in a subject, the method comprising: measuring and/or quantifying and/or determining an amount or level of an analyte in the subject, wherein a change in the amount or level of the analyte in the subject relative to an amount or level of the analyte in a reference sample is indicative of the condition in the subject, wherein the analyte is selected from the group comprising: glutamine, a glutamine metabolite, and any combinations thereof.
5. The method of paragraph 4, wherein the measuring and/or quantifying and/or determining is performed using at least one selected from the group comprising: spectral magnetic resonance imaging, sample assay, and any combinations thereof.
6. The method of paragraph 5, wherein the sample assay is body fluid assay.
7. The method of paragraph 6, wherein the body fluid assay is a blood assay.
8. The method of paragraph 4, wherein the glutamine metabolite is selected from the group comprising: glutamate, glutathione, γ-aminobutyrate, α-ketoglutarate, succinate, fumarate, malate, acetyl-CoA, 2-hydroxyglutarate and any combinations thereof.
9. The method of paragraph 4, wherein the subject has been treated with at least one androgen deprivation therapy.
10. A method for detecting a cancer distinguishably from a non-cancer condition in a subject, comprising: measuring and/or quantifying and/or determining an amount or level of an analyte in the subject; comparing the amount or level of the analyte in the subject to an amount or level of the analyte in a reference sample, wherein a change in the amount or level of the analyte in the subject relative to an amount or level of the analyte in a reference sample is used to distinguish the cancer from the non-cancer condition in the subject, wherein the analyte is selected from the group comprising: glutamine, a glutamine metabolite, and any combinations thereof.
11. The method of paragraph 10, wherein the change in the amount or level of glutamine in the subject is an increase in the amount or level of glutamine in the subject relative to the amount or level of glutamine in the reference sample.
12. The method of paragraph 11, wherein the change in the amount or level of glutamine in the subject with prostate cancer and the concentration of glutamine contributes to the distinction of indolent and aggressive disease.
13. A method of inhibiting progression of prostate cancer in a subject, comprising: administering at least one androgen deprivation therapy to the subject; and administering a therapeutically effective amount of at least one inhibitor to the subject, wherein the at least one inhibitor is selected from the group comprising: at least one macropinocytosis inhibitor, at least one glutamine metabolism inhibitor, at least one glutaminase inhibitor, and combinations thereof.
14. A method for treating depression in a subject, comprising: administering a therapeutically effective amount of at least one inhibitor to the subject, wherein the at least one inhibitor is selected from the group comprising: at least one macropinocytosis inhibitor, at least one glutamine uptake inhibitor, at least one glutamine metabolism inhibitor, at least one glutaminase inhibitor, and any combinations thereof, wherein the subject has a cancer.
15. The method of paragraph 14, wherein the subject is receiving at least one cancer therapy.
16. The method of paragraph 15, wherein the at least one cancer therapy is at least one selected from the group comprising: androgen deprivation therapy, radiation therapy, chemotherapy, and any combinations thereof.
17. The method of paragraph 14, wherein the cancer is prostate cancer, and the subject is receiving a cancer therapy, wherein the cancer therapy comprises at least one androgen deprivation therapy.
18. A method for sensitizing a cancer in a subject to at least one cancer therapy, comprising: administering a therapeutically effective amount of at least one inhibitor to the subject, wherein the at least one inhibitor is selected from the group comprising: at least one macropinocytosis inhibitor, at least one glutamine metabolism inhibitor, at least one glutaminase inhibitor, and any combinations thereof.
19. The method of paragraph 18, where at least one cancer therapy is one selected from the group comprising: androgen deprivation therapy, radiation therapy, chemotherapy, and any combinations thereof.
20. A method for preventing and/or reducing activation of stellate cells in a subject, the method comprising: administering a therapeutically effective amount of at least one inhibitor to the subject, wherein the at least one inhibitor is selected from the group comprising: at least one macropinocytosis inhibitor, at least one glutamine metabolism inhibitor, at least one glutaminase inhibitor, and combinations thereof.
21. The method of paragraph 20, wherein the stellate cells are selected from the group comprising: hepatic stellate cells, pancreatic stellate cells, and any combinations thereof.
22. A pharmaceutical composition, comprising: at least one inhibitor selected from the group comprising: at least one macropinocytosis inhibitor, at least one glutamine metabolism inhibitor, at least one glutaminase inhibitor, and combinations thereof.
23. The pharmaceutical composition of paragraph 22, further comprising at least one androgen deprivation therapy agent.
24. The pharmaceutical composition of paragraph 23, wherein at least one androgen deprivation therapy agent is at least one antiandrogen.
25. The pharmaceutical composition of paragraph 24, wherein the at least one antiandrogen is at least one selected from the group comprising: AR antagonist, androgen synthesis inhibitor, antigonadotropin, and combinations thereof.
26. The pharmaceutical composition of paragraph 23, wherein the at least one androgen deprivation therapy agent is at least one selected from androgen receptor signaling inhibitor, androgen receptor inhibitor, and combinations thereof.
27. The pharmaceutical composition of paragraph 22, wherein the glutamine metabolism inhibitor is selected from the group comprising: L-gamma-glutamyl-p-nitroanilide (GPNA) and analogs, homologs, derivatives, and salts thereof.
28. The pharmaceutical composition of paragraph 22, wherein the glutaminase inhibitor is selected from the group comprising: bis-2(5-phenylacetamido-1,3,4-thiadiazol-2-yl)ethyl sulfide (BPTES) and analogs, homologs, derivatives, and salts thereof, and 2-(pyridin-2-yl)-N-(5-(4-(6-(2-(3-(trifluoromethoxy)phenypacetamido)pyridazin-3-yl)butyl)-1,3,4-thiadiazol-2-yl)acetamide (CB-839) and analogs, homologs, derivatives, and salts thereof.
29. A method for assessing and/or determining the risk of developing a condition in a subject, the method comprising: measuring and/or quantifying and/or determining an amount or level of analyte in the subject, wherein a change in the amount or level of analyte in the subject relative to an amount or level of analyte in a reference sample is indicative of an increased risk of the subject developing the condition and/or disease.
30. A marker for detecting a condition in a subject, comprising at least one selected from the group comprising: glutamine, glutamine metabolite, and any combinations thereof.
31. The marker of paragraph 30, wherein the condition is selected from the group comprising: cancer, depression, liver fibrosis, pancreatic fibrosis, and any combinations thereof.
31. The marker of paragraph 30, wherein the condition is cancer.
33. The marker of paragraph 31, wherein the cancer is a Ras mutated cancer.
34. The marker of paragraph 30, wherein the cancer is selected from the group comprising: adenocarcinoma, neuroendocrine cancer, and any combinations thereof.
35. The marker of paragraph 30, wherein the cancer is selected from the group comprising: prostate cancer, pancreatic cancer, breast cancer, glioblastoma, lung cancer, colon cancer and any combinations thereof.
36. The marker of paragraph 30, wherein the glutamine metabolite is selected from the group comprising: glutamate, glutathione, γ-aminobutyrate, α-ketoglutarate, succinate, fumarate, malate, acetyl-CoA, 2-hydroxyglutarate and any combinations thereof.
37. The method of paragraph 1, further comprising administering at least one additional therapy, wherein the additional therapy is selected from the group comprising: radiation therapy, chemotherapy, and any combinations thereof.
38. The method of paragraph 37, wherein the chemotherapy comprises administering at least one chemotherapy agent.
39. The method of paragraph 1, wherein administration of the at least one inhibitor is before, after, or simultaneous to administration of the androgen deprivation therapy.
40. A method of treating, reducing the severity of and/or slowing the progression of a condition in a subject, comprising: administering a therapeutically effective amount of at least one inhibitor selected from the group comprising: at least one macropinocytosis inhibitor, at least one glutamine metabolism inhibitor, at least one glutaminase inhibitor, and any combinations thereof, thereby treating, reducing the severity of and/or slowing the progression of the condition in the subject.
41. A method of inhibiting progression of a condition in a subject, comprising: administering a therapeutically effective amount of at least one inhibitor selected from the group comprising: at least one macropinocytosis inhibitor, at least one glutamine metabolism inhibitor, at least one glutaminase inhibitor, and any combinations thereof, thereby inhibiting the progression of the condition in the subject.
42. A method for assessing and/or determining the risk of developing a condition and/or disease in a subject, the method comprising: measuring and/or quantifying and/or determining an amount or level of an analyte in the subject, wherein a change in the amount or level of the analyte in the subject relative to an amount or level of the analyte in a reference sample is indicative of an increased risk of the subject developing the condition and/or disease, wherein the analyte is selected from the group comprising: glutamine, a glutamine metabolite, and any combination thereof.
43. A composition for treating a condition in a subject, wherein the composition comprises: at least one inhibitor selected from the group comprising: at least one macropinocytosis inhibitor, at least one glutamine uptake inhibitor, at least one glutamine metabolism inhibitor, at least one glutaminase inhibitor, and any combinations thereof.
44. The composition of paragraph 43, wherein the condition is selected from the group comprising: cancer, tumor, liver fibrosis, pancreatic fibrosis, and any combinations thereof.
45. The composition of paragraph 43, wherein the condition is cancer.
46. The composition of paragraph 45, wherein the cancer is a Ras mutated cancer.
47. The composition of paragraph 45, wherein the cancer is selected from the group comprising: adenocarcinoma, neuroendocrine cancer, and any combinations thereof.
48. The composition of paragraph 45, wherein the cancer is selected from the group comprising: prostate cancer, pancreatic cancer, breast cancer, lung cancer, colon cancer, and any combinations thereof.
49. The composition of paragraph 43, wherein the composition further comprises at least one androgen deprivation therapy agent.
50. A method for prognosing a condition in a subject, the method comprising: measuring and/or quantifying and/or determining an amount or level of analyte in the subject, wherein a change in the amount or level of analyte in the subject relative to an amount or level of analyte in a reference sample is a prognosis of the condition in the subject, wherein the analyte is selected from the group comprising: glutamine, a glutamine metabolite, and any combinations thereof.
51. The method of any one of paragraphs 4, 29, 30, 40, 41, 43, or 50, wherein the condition is selected from the group comprising: cancer, depression, liver fibrosis, pancreatic fibrosis, and any combinations thereof.
52. The method of any one of paragraphs 4, 29, 30, 40, 41, 43, or 50, wherein the condition is cancer.
53. The method of paragraph 18, wherein the cancer is a Ras mutated cancer.
54. The method of paragraph 18, wherein the cancer is selected from the group comprising: adenocarcinoma, neuroendocrine cancer, and any combinations thereof.
55. The method of paragraph 18, wherein the cancer is selected from the group comprising: prostate cancer, pancreatic cancer, breast cancer, lung cancer, colon cancer, and any combinations thereof.
56. The method of paragraph 1, wherein the at least one androgen depravation therapy comprises administering a therapeutically effective amount of at least one androgen deprivation therapy agent.
57. The method of paragraph 56, wherein the at least one androgen deprivation therapy agent is at least one antiandrogen.
58. The method of paragraph 57, wherein the at least one antiandrogen is at least one selected from the group comprising: AR antagonist, androgen synthesis inhibitor, antigonadotropin, and any combinations thereof.
59. The method of paragraph 56, wherein the at least one androgen deprivation therapy agent is at least one selected from androgen receptor signaling inhibitor, androgen receptor inhibitor, and any combinations thereof.
60. The method of any one of paragraphs 1, 13, 14, 18, 20, 40, or 41, wherein the glutamine metabolism inhibitor is selected from the group comprising: L-gamma-glutamyl-p-nitroanilide (GPNA) and analogs, homologs, derivatives, and salts thereof.
61. The method of any one of paragraphs 1, 13, 14, 18, 20, 40, or 41, wherein the glutaminase inhibitor is selected from the group comprising: bis-2(5-phenylacetamido-1,3,4-thiadiazol-2-yl)ethyl sulfide (BPTES) and analogs, homologs, derivatives, and salts thereof, and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839) and analogs, homologs, derivatives, and salts thereof.
62. The method of any one of paragraphs 10, 29, or 50 wherein the measuring and/or quantifying and/or determining is performed using at least one selected from the group comprising: spectral magnetic resonance imaging, sample assay, and any combinations thereof.
63. The method of paragraph 5, wherein no radioactive tracer or contrast agent is administered to the subject.
64. The method of any one of paragraphs 4, 10, 29, or 50, wherein the reference sample is obtained from a control subject, wherein the control subject does not have the condition or disease.
65. The method of any one of paragraphs 4, 10, 29, or 50, wherein the reference sample is obtained from the subject before the subject is treated for the condition or disease.
66. The method of any one of paragraphs 4, 10, 29, or 50, wherein the reference sample is from a subject that has been successfully treated for the condition or disease.
67. The method of any one of paragraphs 4, 10, 29, or 50, wherein the change in the amount or level of glutamine and/or a glutamine metabolite in the subject is an increase in the amount or level of glutamine and/or the glutamine metabolite in the subject relative to the amount or level of glutamine and/or the glutamine metabolite in the reference sample.
68. The method of any one of paragraphs 4, 10, 29, or 50, wherein the change in the amount or level of glutamine and/or a glutamine metabolite in the subject is a decrease in the amount or level of glutamine and/or the glutamine metabolite in the subject relative to the amount or level of glutamine and/or the glutamine metabolite in the reference sample.
69. The method of any one of paragraphs 4, 10, 29, or 50, wherein the condition is androgen deprivation therapy resistance.
70. The method of paragraph 1, wherein the at least one androgen deprivation therapy comprises at least one selected from at least one surgical technique, at least one androgen deprivation therapy agent, and any combinations thereof.
71. The composition of paragraph 43, wherein the condition is androgen deprivation therapy resistance.

### EXAMPLES

The present disclosure will be further explained by the following Examples, which are intended to be purely exemplary of the invention and should not be considered as limiting the invention in any way. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

Cell Culture. Prostate fibroblasts from prostate cancer patients and mouse prostate were cultured in high glucose Dulbecco's Modified Eagle Medium (DMEM, Gibco/Life Technologies) supplemented with 5% fetal bovine serum (Gibco/Life Technologies) and Antibiotic-Antimycotic (Gibco/Life Technologies) at 37° C. and 5% CO₂. In order to test the purity of CAFs and NAFs, we examined fibroblast biomarkers in these cells. CW22Rv1 and C42B cells (androgen-independent human PCa cell line), were maintained in RPMI-1640 medium with 5% fetal bovine serum and 1% penicillin/streptomycin. All cell lines were obtained from the American Type Culture Collection. All cultures were grown in a humidified 5% CO₂ environment at 37° C.

DNA methylation analysis. Libraries were prepared from 200-500 ng of genomic DNA digested with 60 units of Taqα1 and 30 units of Mspl (NEB) sequentially and then extracted with Zymo Research DNA Clean & Concentrator™-5 kit. Fragments were ligated to pre-annealed adapters containing 5′-methyl-cytosine instead of cytosine according to Illumina's specified guidelines (www.illumina.com). Adaptor-ligated fragments of 150-250 bp and 250-350 bp in size were recovered from a 2.5% NuSieve 1:1 agarose gel (Zymo Research). The fragments were then bisulfite-treated using the EZ DNA Methylation-Lightning™ Kit. Preparative-scale PCR was performed and the resulting products were purified for sequencing on an Illumina HiSeq. (Further details of DNA methalome analysis provided in the Examples Section herein.).

Bisulfite Sequencing. Bisulfite sequencing was performed as described previously (Haldar S, Dru C, Mishra R, Tripathi M, Duong F, Angara B, Fernandez A, Arditi M, and Bhowmick N A. Histone deacetylase inhibitors mediate DNA damage repair in ameliorating hemorrhagic cystitis. Sci Rep. 2016; 6(39257). Briefly, genomic DNA was extracted from the NAF and CAF samples and 200 ng of DNA was bisulfite converted by using the EZ DNA Methylation-Gold Kit (Zymo Research, Irvine, Calif.) according to the manufacturer's protocol. Three overlapping primers were used for amplification of Exon 2 region of RASAL3 by ZymoTaq DNA polymerase (Zymo Research) and subsequently cloned into the pCR2.1-TOPO vector (Invitrogen by Life Technologies, Carlsbad, Calif.). The primer sequences used to amplify the three regions were designed by using the Meth-Primer program (www.urogene.org/methprimer/) and are listed in Table 4. Five clones from each sample were sequenced by using Sanger's method.

Macropinocytosis assay. These were performed as described previously (Commisso C, Davidson S M, Soydaner-Azeloglu R G, Parker S J, Kamphorst J J, Hackett S, Grabocka E, Nofal M, Drebin J A, Thompson C B, et al. Macropinocytosis of protein is an amino acid supply route in Ras-transformed cells. Nature. 2013; 497(7451):633-7), briefly fibroblasts which were grown on cover slips, serum-starved for 18 hours. TMR-dextran (1 mg/ml; 70 kDa, FITC-labeled), which is an established marker of macropinocytosis, was added to serum-free medium for 30 min at 37° C. for macropinosomes labeling (Commisso C, Davidson S M, Soydaner-Azeloglu R G, Parker S J, Kamphorst J J, Hackett S, Grabocka E, Nofal M, Drebin J A, Thompson C B, et al. Macropinocytosis of protein is an amino acid supply route in Ras-transformed cells. Nature. 2013; 497(7451):633-7). After removing the dextran containing medium, cells were rinsed five times in cold PBS and immediately fixed in 3.7% formaldehyde. Coverslips mounted in DAPI containing mounting medium (Vectashield) onto slides. Images were captured using laser confocal scanning microscopy (Leica) and analyzed via LASX software. To verify that macropinocytosis co localizes with lysosomes, LysoTracker Red, as the indicator of lysosome, was added in cells along with TMR dextran for 30 min. The fluorescence signals in the cells were imaged using FSX100 (Olympus, USA). To determine whether the internalized albumin is intracellularly degraded, we have utilized a self-quenching red fluorescent dye, where lysosomal proteases enable red fluorescence visualization. Cells were incubated in RPMI media containing DQ-BSA (10 μg/ml) for 30 min and washed twice with PBS and monitored by confocal microscopy.

3D organotypic co-culture. A modified version of the 3D organotypic co-culture system was performed in a collagen matrix similar to that previously reported (Stark H J, Baur M, Breitkreutz D, Mirancea N, and Fusenig N E. Organotypic keratinocyte cocultures in defined medium with regular epidermal morphogenesis and differentiation. J Invest Dermatol. 1999; 112(5):681-91). Matrix were prepared by mixing five volumes of rat tail collagen with two volumes of matrigel in one volume of 10×DMEM medium (GE Healthcare Life Sciences), and one volume of FBS (Atlanta Biologicals). CWR22Rv1 and primary mouse prostatic fibroblasts (WT or RAS^{V12}) were combined in a 1:3 ratio in the matrix. Nylon squares were coated with collagen and placed on metal grids in a 6-well plate. Matrix plugs containing cells, formed in 96 well plates (150 μl), were transferred onto the nylon squares and media was added to the level of the nylon mesh. Following treatments, the cells were dissociated from the matrix with collagenase and dispase for FACS analysis.

Seahorse XF-24 metabolic flux analysis. Oxygen consumption rate (OCR, pmol min-1) was measured with the Searhorse Bioscience XF24 extracellular flux analyzer. Cells were incubated in a CO₂-free incubator for 1 h at 37° C. to allow for temperature and pH equilibration prior to loading into the XF24 apparatus. Initial assays were performed to optimize cell number, FCCP concentration and oligomycin concentration (data not shown). We used L-Glutamic acid γ-(p-nitroanilide) hydrochloride (GPNA, 10 μM), a specific glutamine exporter inhibitor.

Tissue recombination mouse models. Cell recombinants were prepared by mixing 2×10⁵ epithelial (CWR22Rv1) cells with 6×10⁵ CAFs or stromal cells expressing KRAS^{V12} per site in collagen as described before (Banerj ee J, Mishra R, Li X, Jackson R S, 2nd, Sharma A, and Bhowmick N A. A reciprocal role of prostate cancer on stromal DNA damage. Oncogene. 2014; 33(41):4924-31). The mice were either left intact or castrated 1 week after grafting. The intact mice were randomly divided into two groups, where one received 3 mg/kg 5-(N-Ethyl-N-isopropyl) amiloride (EIPA; Sigma-Aldrich) intraperitoneal injection or vehicle control every other day for one week. Castrated mice were treated with enzalutamide (5 mg/kg) either alone or in combination with L-glutamic acid Υ-(p-nitroanilide) hydrochloride (GPNA; 10 mg/kg; Sigma-Aldrich) for three weeks. The tumor volumes were measured using digital calipers and calculated using the following formula: length (mm)×width (mm)×width (mm)×0.52. When the mice were sacrificed and tumors were excised and photographed. Formalin-fixed, paraffin-embedded (FFPE) xenograft tumor tissue was sectioned and stained with hematoxylin and eosin (H&E) and staining for chromogranin A, TUNEL, phosphorylated-hi stone H3 and phosphorylated-mTOR.

Spectral magnetic resonance for glutamine/glutamate. Chemical exchange saturation transfer effect of amine protons was used to image glutamine and glutamate, using a 9.4T micro-MRI in orthotopic prostate xenografts in mice, similar to that previously described (Cai K, Haris M, Singh A, Kogan F, Greenberg J H, Hariharan H, Detre J A, and Reddy R. Magnetic resonance imaging of glutamate. Nat Med. 2012; 18(2):302-6). The mice were imaged before and one hour after intraperitoneal administration of the macropinocyosis inhibitor 5-(N-ethyl-N-isopropyl)amiloride (EIPA).

Statistics. Reduced Representation Bisulfite Sequencing BaseClear (Netherlands) performed quality control, sequence processing and mapping of sequence reads as per their ‘EpiQuest Genome-wide Basic’ service package. Sequence depth was determined per CpG dinucleotide as the number of reads where methylation status could be determined and CpGs with a depth less than 10 reads in either condition was excluded. Methylation status of the remaining CpGs was calculated as the percentage of methyl-cytosine in total cytosine. Heatmap was made using metaboanlyst (www.metaboanalyst.ca) software using top 25 genes analyzed by ANOVA.

Statistical significance of differences between control and experimental groups was assessed by unpaired Student's t test; for multiple comparison, one-way ANOVA was followed by Sidak multiple comparison tests. The repeated-measures two-way ANOVA with post hoc analyses was also used where appropriate. P values of less than 0.05 were considered statistically significant. Data presented in Fig. legends is represented as mean±SEM unless otherwise stated; statistical tests utilized are reported in the Fig. legends, along with the associated P values.

Study approvals. All mouse studies were approved and performed by approved Cedars-Sinai Animal Care and Use Committee protocol. All PCa patients treated with ADT in this study, detailed in Table 1, were consented for blood analysis under Institutional Review Board of Cedars-Sinai Medical Center.

Lentiviral Infections in mouse stromal cells. Lentiviral transductions of the empty, GFP expressing vector and Ras^{V12} gene in wild type mouse prostatic fibroblasts in the presence of 5.0 μg/mL polybrene (Sigma-Aldrich). After stable clones were selected under hygromycin (50 μg/mL) selection, Ras expression was verified by western blotting.

RNA expression level. All real-time PCR assays were carried out using three technical replicates and three independent cDNA syntheses on a 7500 Real-Time PCR System (Applied Biosystems). Data were normalized using an internal control gene, either GAPDH or β-actin. Relative quantification of expression levels (RQ) was calculated using the ^{2-ΔΔCt} method. Primers used for qPCR are listed in Table 4.

Western blots. Western blots performed with 4-12% SDS-polyacrylamide gels. Following electrophoresis gels were transferred to PVDF membrane (BioRad) in transfer buffer (25 mM Tris; 200 mM glycine; 20% methanol v/v). Membranes were blocked with PBS (phospho-buffered saline) with 0.1% Tween20 (Sigma) and 5% non-fat dry milk or Bovine serum albumin (PBST-milk) for at least 45 min. Detection was performed using alkaline phosphatase-conjugated secondary antibodies (Sigma-Aldrich). Antibody information are listed in Table 5. Experiments were repeated in at least three independent experiments, and one of the representative blots was shown.

Active Ras pull-down assay. Ras activity was detected using a Ras Activation Assay Kit (Thermo Fisher Scientific) following the manufacturer's instructions using 500 μg protein for each condition. Ras activity was reflected by the amount of Ras-GTP pulled down by Raf1 RBD (Ras-binding domain) relative to total Ras expression by western blotting.

ATP assay. CWR22Rv1 cells (1×10⁵) were plated into a 6-cm culture dish and allowed to adhere. After 24 hr, the medium was replaced with conditioned media obtained from primary mouse wild type or Ras^{V12} prostatic fibroblasts as well as primary human NAF or CAF. Controls included vehicle (DMSO) and oligomycin (2 μM). After 72 hr incubation with the conditioned media, the cells were lysed for measurement of intracellular ATP levels using an ATP determination kit (Thermo Fisher) according to the manufacturer's instructions. Luminescence was measured with Monolight 3010 luminometer (Pharmingen, San Diego, Calif.). All values were normalized to total protein, and the cellular ATP level is expressed as nanomole per microgram of protein.

Metabolome analysis. CWR22Rv1 cells were grown in NAF or CAF conditioned media for 72 hr. Cell pellets were flash frozen in liquid nitrogen for untargeted metabolic profiling of known and unknown metabolites using liquid chromatrography-mass spectrometry by Metabalon Inc. as previously described (Evans A M, DeHaven C D, Barrett T, Mitchell M, and Milgram E. Integrated, nontargeted ultrahigh performance liquid chromatography/electrospray ionization tandem mass spectrometry platform for the identification and relative quantification of the small-molecule complement of biological systems. Anal Chem. 2009; 81(16): 6656-67.).

Measurements of glutamine. Glutamine was measured in blood plasma and conditioned media by using glutamine detection kit (Abcam) following manufacturer protocol. Deproteinization step was performed in plasma samples prior to glutamine detection. The absorbance was measured at 450 nm using a Bio-Rad Benchmark plus microplate spectrophotometer (Bio-Rad Laboratories).

TCGA analysis. Gene expression data were downloaded from the TCGA GDAC Firehose portal [Broad Institute TCGA Genome Data Analysis Center (2016), Firehose stddata_2016_01_28 run. Broad Institute of MIT and Harvard. doi:10.7908/C11G0KM9]. For analyzing PHB expression, the normalized RSEM counts were used after log 2 transformation. RASAL3 genomic sequencing status was retrieved from the UCSC Cancer Genomics Browser (genome-cancer.ucsc.edu/).

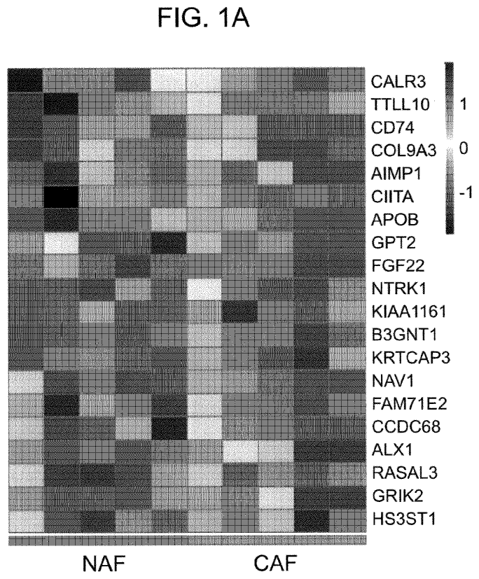

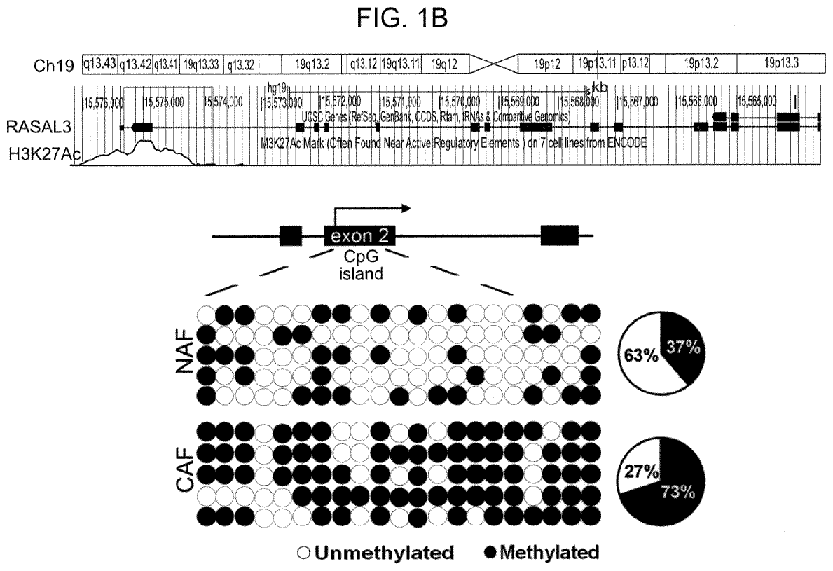

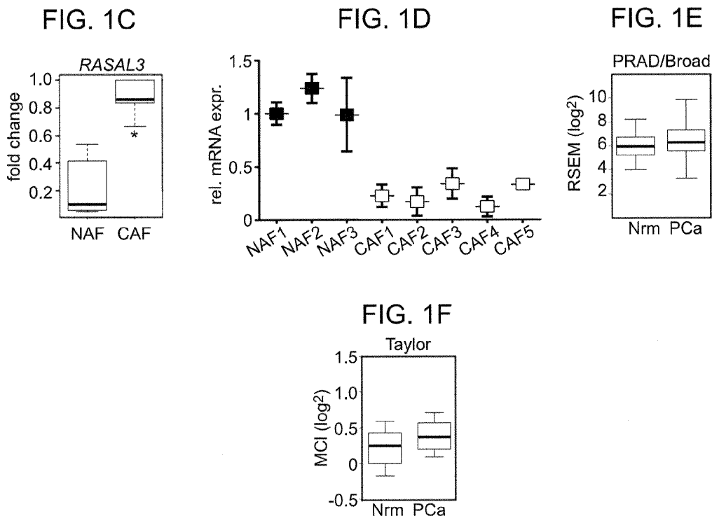

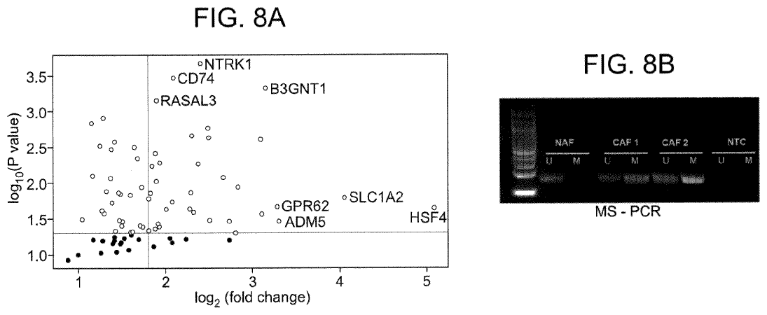

DNA methylome analysis revealed epigenetic silencing of RASAL3 in prostatic CAF. In order to identify differences in the methylation status of prostate fibroblasts from human benign and PCa tissues, we performed whole-genome methylation analysis by Reduced Representation Bisulfite Sequencing (RRBS). Sequencing data from 10 primary prostatic NAF and CAF samples in a pairwise fashion were analyzed to determine differentially methylated CpG sites. The RRBS analysis led to a list of more than 2000 genes that displayed differential promoter methylation in the NAF and CAF populations (Table 7). Among the top 20 hypermethylated promoters in CAF, four genes appeared to top the list and were tested: CD74, RASAL3, B3GNT1, and NTRK1 (see, e.g., FIG. 1A and FIG. 8A). Out of the four genes, CD74, NTRK1 (Neurotrophic Receptor Tyrosine Kinase 1), and B3GNT1 (Beta-1,4-Glucuronyltransferase 1) part of glycosaminoglycan metabolism pathway, have well studied roles in promoting tumor progression (Afratis N, Gialeli C, Nikitovic D, Tsegenidis T, Karousou E, Theocharis A D, Pavao M S, Tzanakakis G N, and Karamanos N K. Glycosaminoglycans: key players in cancer cell biology and treatment. FEBS J. 2012; 279(7):1177-97; Schroder B. The multifaceted roles of the invariant chain CD74—More than just a chaperone. Biochim Biophys Acta. 2016; 1863 (6 Pt A):1269-81; Vaishnavi A, Capelletti M, Le A T, Kako S, Butaney M, Ercan D, Mahale S, Davies K D, Aisner D L, Pilling A B, et al. Oncogenic and drug-sensitive NTRK1 rearrangements in lung cancer. Nat Med. 2013; 19(11):1469-72). However, RASAL3 (RAS protein activator like 3) is a tumor suppressor, as a RAS signaling antagonist, located at the 19p13.12 locus demonstrated exon 2 hypermethylation in CAF (p<0.002; see, e.g., FIG. 1B and FIG. 1C). We selected RASAL3 as a candidate gene for further study, as promoter hypermethylation and silencing of a tumor suppressor in CAF may have a tumorigenic effect.

To confirm the methylation status of the RASAL3 promoter, methylation-specific PCR and bisulfite genomic sequencing were performed in NAF and CAF primary lines. Greater exon 2 methylation was found in the CAF compared to the NAF, as determined by methylation-specific PCR (see, e.g., FIG. 8B). Bisulfite sequencing of exon 2 demonstrated 73.3% CpG dinucleotide methylation of RASAL3 in CAF, compared to 37.1% CpG methylation in NAF, confirming the methylome analysis (see, e.g., FIG. 1C). Accordingly, RASAL3 mRNA expression was found to be significantly downregulated in CAF from four patients compared to NAF from three patients, as determined by rtPCR (see, e.g., FIG. 1D). However, Oncomine analysis demonstrated no significant difference in RASAL3 expression in normal and PCa tumor tissues from either PRAD/Broad and Taylor data sets (see, e.g., FIG. 1E and FIG. 1F). Thus, RASAL3 is prominently epigenetically silenced in human CAF compared to NAF, but is not differentially expressed in the epithelial compartment, enriched in the two data sets.

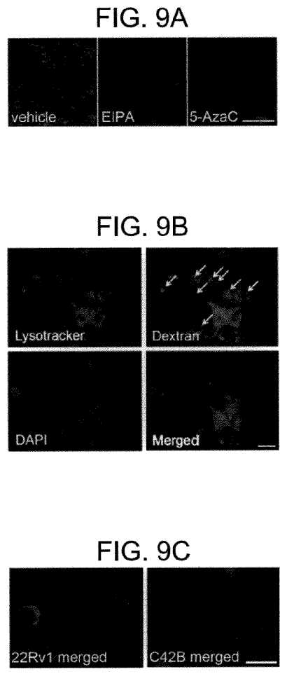

Ras signaling activation in prostatic fibroblast leads to macropinocytosis. Since the RasGAPs are negative regulators of Ras and their loss contributes to Ras pathway activation, we tested Ras activity in CAF and NAF. We found that RASAL3 protein expression was reduced in a primary CAF population, accompanied by a substantial increase in Ras-GTP (active Ras), phosphorylated-c-Raf, phosphorylated-ERK, compared to that in NAF (FIG. 2A). Total Ras and cRaf protein expression remained unchanged in NAF and CAF. We reasoned that the role of epigenetically-induced Ras activation in CAF could trigger an endocytic process of macropinocytosis similar to that reported in pancreatic cancer epithelia (Commisso et al., 2013). The uptake of fluorescent dextran beads (TMR-70 kDa dextran) in NAF and CAF populations were visualized. Interestingly, dextran labeling experiments illustrated a large number of macropinosomes in CAF, with no detectible uptake of fluorescence in the NAF (FIG. 2B). TMR-dextran uptake by CAF was inhibited by the macropinocytosis inhibitor, 5-(N-Ethyl-N-isopropyl) amiloride (EIPA), as well as the DNA demethylase, 5-aza-2′-deoxycytidine, in support of epigenetic regulation of macropinocytosis in these fibroblasts (FIG. 9A). We further found that macropinosomes in CAF trafficked to lysosomes while undergoing a process of maturation characterized by vesicular size reduction concomitant with membrane fusion visualized by co-localization of TMR-dextran and lysotracker (FIG. 9B). To identify albumin proteolysis in CAF, we used self-quenching BODIPY dye conjugated to BSA (DQ-BSA) that emits green fluorescence following proteolytic digestion. The co-localization of TMR-dextran and DQ-BSA indicated degraded BSA was compartmentalized in macropinocytic vesicles within CAF (FIG. 2C). The epigenetic silencing of RASAL3 in CAF correlated with Ras-mediated macropinocytosis and subsequent degradation of BSA.

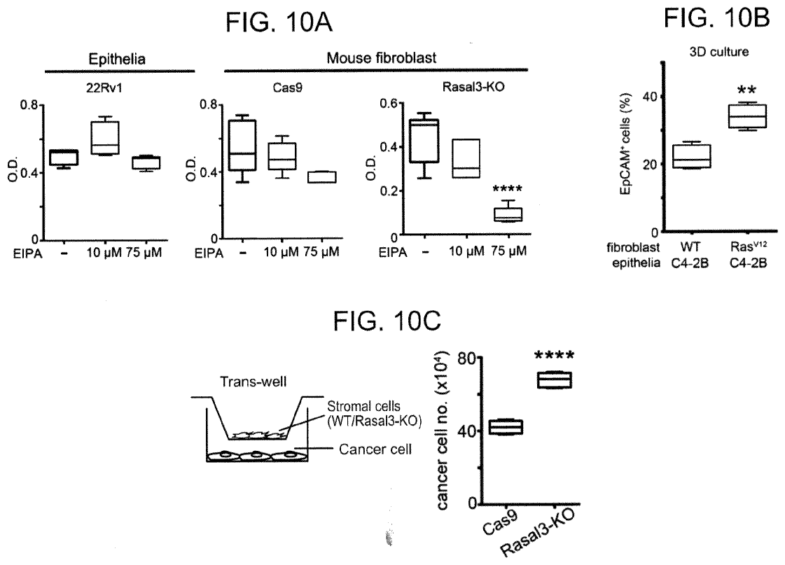

To directly determine if Ras signaling confers a tumor supportive role associated with CAF, we used CRISPR/Cas9 to knock out Rasa13 in mouse prostatic fibroblasts. Compared to the expression of Cas9 fibroblasts (control), Ras activity and ERK phosphorylation was elevated in Rasa13-knockout (Rasa13-KO) fibroblasts (FIG. 2D). Rasa13-KO fibroblasts took up TMR dextran, whereas it was undetectable in the control fibroblasts (FIG. 2E). The exogenous expression of active Ras^{V12} (Gly¹²→Val¹²) in wild-type mouse prostatic fibroblasts replicated the downstream ERK activation and macropinocytosis observed in CAF and Rasa13-KO cells (FIG. 2 F,G). Thus, epigenetic RASAL3 silencing of prostatic fibroblasts promoted familiar signaling and macropinocytosis associated with Ras transformation (Commisso et al., 2013). Conversely, PCa epithelial cell lines, CWR22Rv1 and C4-2B, were not found to exhibit macropinocytosis, based on TMR-dextran uptake (FIG. 9C). To test the role of stromal Ras-mediated macropinocytosis on tumor development, orthotopic PCa xenograft models were used. Tissue recombinants of Ras^{V12}-expressing fibroblasts and CWR22Rv1 epithelia were grafted into the anterior prostates of SCID mice. Two weeks later, mice were treated with vehicle control or EIPA for 1 week to suppress macropinocytosis (FIG. 2H). We found that the mice treated with EIPA had dramatically reduced tumor size with respect to the control group. Treatment with EIPA had no significant effect on the viability of the CWR22Rv1 or wild type fibroblasts, but EIPA reduced the viability of the Rasa13-KO fibroblasts (P<0.001, FIG. 10A). Thus, glutamine uptake by the tumor was necessary for its expansion.

Uptake of fibroblast-derived glutamine by prostate cancer epithelia impacts its metabolism. To establish the role of Ras on the reactive properties of fibroblasts, we tested its role on the expression of a panel of CAF-defining genes. Ras^{V12}-fibroblasts had 2.5- to 140-fold elevated expression of a panel of CAF marker-genes, compared to wild type fibroblasts, inclusive of tenascin C, FAP, MMP1a, and MMP3 (two-way ANOVA P<0.0001, FIG. 3A). Because reactive fibroblasts are recognized to potentiate adjacent epithelial expansion, epithelial proliferation was tested in three-dimensional (3D) co-cultures with fibroblasts. PCa epithelia, CWR22Rv1 and C4-2B, were found to be significantly more proliferative when cultured with Ras^{V12}-fibroblasts, compared to wild type fibroblasts (FIG. 2B, FIG. 10B). To determine if a soluble factor was involved in the paracrine process, a transwell assay was performed with Cas9 or Rasa13-KO fibroblasts in the upper chamber and CWR22Rv1 in the lower well (FIG. 10C). We found a significant proliferative induction of the epithelia when associated with Rasa13-KO fibroblasts, compared to Cas9 control fibroblasts. To correlate the uptake of albumin in fibroblasts and elevated Ras activity with epithelial proliferation we tested whether the stromal media affected epithelial ATP generation. Conditioned media (CM) from both Ras^{vll}-fibroblast induced ATP production in CWR22Rv1 and C4-2B, over CM from the wild type counterpart (FIG. 3C, FIG. 11A). Similar ATP generation was observed in CWR22Rv1 in the context of CAF-CM, compared to NAF-CM. The ATP synthase inhibitor, oligomycin, served as the negative control for ATP detection. These results suggested that Ras-induced macropinocytosis in stromal fibroblasts confer a change in tumor-energy metabolism via a paracrine soluble factor.

As it was clear that epigenetically transformed stromal fibroblasts provided the energy needs of the epithelia, the nature of these metabolic changes was explored. Metabolome analysis of CWR22Rv1 demonstrated glutamine and glutamate to be significantly elevated when exposed to CAF-CM, compared to NAF-CM, (P<0.01, FIG. 3D). This was consistent with macropinocytosis of albumin, as previously reported in pancreatic cancer (Davidson et al., 2017). Consequently, succinate, fumarate and malate levels were significantly elevated in the CAF-CM treated group, compared to that induced by NAF-CM, consistent with glutamine metabolism. However, there was no significant difference in the levels of acetyl-CoA, α-ketoglutarate, and 2-hydroxyglutarate, but decreased levels of citrate in the epithelia treated with CM from CAF compared to NAF. The lack of a difference in α-ketoglutarate levels could be a result of increased flux to its metabolites (i.e. succinate, fumarate, malate). Further, there was an observed increase in levels of the nucleotide precursor aspartate (P<0.05) and oxidized-GSH (P<0.01), metabolites which are downstream of glutamate metabolism (FIG. 3E). CAF-CM, compared to NAF-CM did not significantly change reduced-GSH levels, associated with increased mitochondrial activity and reactive oxygen in cancer cells.

We found that like the CAF, Rasa13-KO and Ras^{V12}-mouse fibroblasts secreted significantly more glutamine into the media than their NAF or wild type counterparts (FIG. 3F, FIG. 11B). To support the role of stromal-derived glutamine on epithelial proliferation, we supplemented 0.4 mM glutamine to NAF-CM, to find it restored CWR22Rv1 proliferation to that similar to CAF-CM, based on direct measurement of glutamine generated by CAF (FIG. 3G). Developments in magnetic resonance imaging technology have enabled glutamine/glutamate imaging with improved spatial and temporal resolution. However, its application has been relegated to glioblastoma/medulloblastoma imaging, as they are commonly Ras-driven cancers with abundance of glutamine and glutamate in the tissue (Cai et al., 2012; Tardito et al., 2015; Wilson et al., 2014). We were able to identify the presence of glutamine/glutamate in a tissue recombination xenograft model of CWR22Rv1 and Ras^{V12}-mouse fibroblasts. To validate the specificity of the glutamine/glutamate signal, macropinocytosis was inhibited by administering EIPA to the mice and re-imaged (using simulated echo acquisition, FIG. 3H). Stromal fibroblastic Ras driven-macropinocytosis results in glutamine production and utilized by cancer epithelia to promote its expansion.

To corroborate the observed epithelial accumulation of glutamine and its downstream metabolites, we tested if CAF-CM regulated the expression of amino acid transporters and glutaminase expression in CWR22Rv1 and C4-2B cells. Specifically, the glutamine importer SLC1A5 mRNA expression was upregulated in CWR22Rv1 (P<0.0001) and C4-2B (P<0.001) by CAF-CM, compared to NAF-CM (FIG. 11C). Supplementing basal media with L-glutamine consistently increased the expression of glutamine importers SLC1A5 and SCL38A2, in both CWR22Rv1 and C4-2B cells in a time dependent manner (FIG. 11D-G). While, the SLC38A1 transporter did not seem to be regulated by exogenous glutamine in either epithelial line, CWR22Rv1 cells expressed SLC38A4 in a glutamine dependent manner. Once the glutamine enters the epithelia, it can undergo anaplerosis by which glutamine is converted to glutamate by glutaminase (i.e. GLS or GLS2) to replenish the TCA cycle. Glutamine uptake was able to promote both GLS and GLS2 mRNA expression within 6 hours of treatment in C4-2B cells (FIG. 11H). We found GLS mRNA expression level was also elevated in the CAF-CM treated group compared with NAF-CM treated group in both CWR22Rv1 (P<0.0001) and C4-2B (P<0.01) cells (FIG. 11I). GLS2 mRNA expression was however downregulated in CWR22Rv1 cell (P<0.001) and upregulated in C4-2B cells (P<0.0001) by CAF-CM treatment when compared to NAF-CM treatment (FIG. 11J). These data demonstrated that glutamine taken up by the epithelia are metabolized through a seemingly positive feedback mechanism to support the elevated energy needs of cancer epithelia (FIG. 11J).

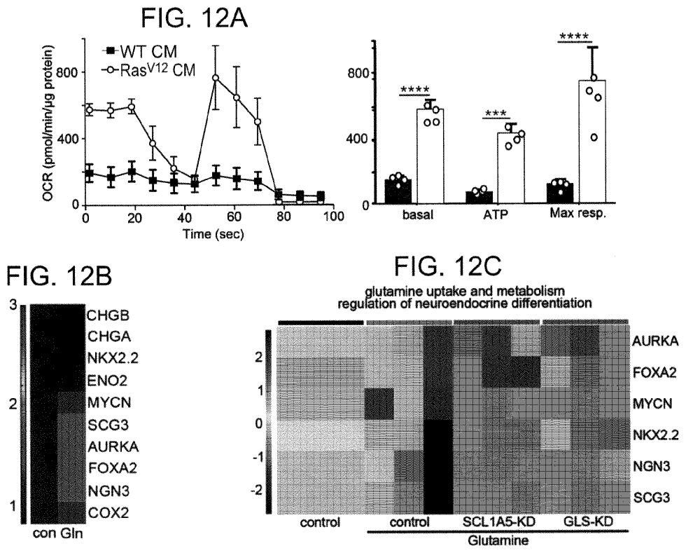

We found that glutamine uptake provided energy and mediated differentiation signaling for the prostatic epithelia. The prostatic CAF potentiated epithelial TCA flux resulting in a mitochondrial response in CWR22Rv1 cells, by the administration of exogenous glutamine (FIG. 4A). Similarly, Ras^{V12}-fibroblast-CM upregulated basal oxygen consumption, ATP generation, and maximal mitochondrial respiration in CWR22Rv1 and C4-2B, compared to that with wild type fibroblast-CM (FIG. 4B, FIG. 12A). Glutamine deprivation by treatment with an SLC1A5 inhibitor, L-γ-glutamyl-p-nitroanilide (GPNA) on CWR22Rv1 and C4-2B in the context of Ras^{V12} fibroblast-CM, restored basal respiration, ATP, and maximal respiration to that observed with wild type fibroblast-CM. To determine the impact of stromal-derived glutamine on epithelial proliferation, we blocked glutamine uptake and utilization with selective inhibitors in 3D co-cultures of primary human CAF and CWR22Rv1 cells. We found that blocking SLC1A5 (i.e., GPNA) and GLS (i.e., bis-2-[5-phenylacetamido-1,2,4-thiadiazol-2-yl] ethyl sulfide: BPTES) function significantly decreased epithelial proliferation, as determined by quantitating Ki-67 staining of EpCAM⁺ cells through FACS analysis (P<0.05 and P<0.001, respectively, FIG. 4C). Thus, the stromal epigenetic modification resulted in glutamine production for the maintenance of ATP levels and proliferation in cancer cells. Given that mTOR is a nutrient sensor for glutamine, we reasoned that the same pathway may affect mechanisms of prostate cancer differentiation (Qian et al., 2013; Wu and Huang, 2007). We found mTOR to be activated in CWR22Rv1 cells incubated with Ras^{V12}-fibroblast-CM (FIG. 4D; Statistical analysis shown in Table 3). Moreover, expression of Forkhead box M1 (FOXM1), a key signal downstream of mTOR and a critical transcription factor in multiple poorly differentiated cancer types (Andersson et al., 2016; Chan et al., 2017; Kalin et al., 2006) was upregulated in CWR22Rv1 cells incubated with Ras^{V12}-fibroblast-CM. FOXM1 was also found to interact with multiple neuroendocrine differentiation factors as shown by STRING analysis (FIG. 4E) (Szklarczyk et al., 2017). Remarkably, the incubation of CWR22Rv1 cells with Ras^{V12}-fibroblast-CM resulted in significant increase in neuroendocrine differentiation genes shown in a heatmap (F-test P<0.0001, FIG. 4F). Similarly, the addition of L-glutamine alone upregulated a panel of neuroendocrine gene panel in CWR22Rv1 epithelia (F-test P<0.0001), with the inhibition of mTOR by rapamycin restoring its expression to control levels (FIG. 4G). The sufficiency for glutamine alone to induce neuroendocrine differentiation was further confirmed in C4-2B cells (P<0.0001, FIG. 12B). Finally, we found that the knockdown of either SLC1A5 or GLS by siRNA was able to reverse the effect of glutamine-induced neuroendocrine differentiation of CWR22Rv1 (FIG. 12C). Taken together, these results demonstrated that stromal glutamine was sufficient and necessary for epithelial proliferation and differentiation to a more aggressive PCa phenotype mediated by stromal Ras activity.

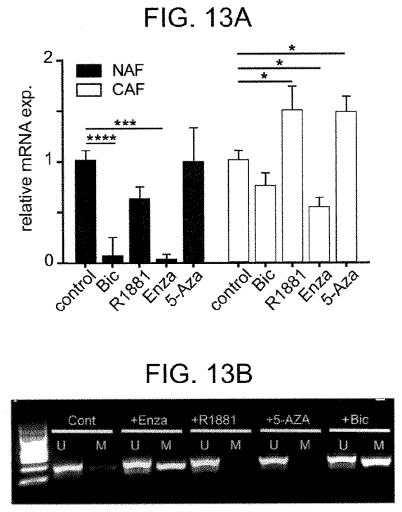

Epigenetic alteration of RASAL3 mRNA expression by androgen signaling affects glutamine driven tumor progression. Neuroendocrine PCa is rare, however neuroendocrine differentiation is more commonly observed as a potential adaptive response to newer, more potent ADT (Beltran et al., 2014). To explore the possible involvement of androgen signaling on prostatic stromal epigenetic alteration, we initially measured RASAL3 mRNA expression in the context of androgen and androgen receptor antagonists. As before, we found that NAF populations had greater RASAL3 mRNA expression compared to CAF (FIG. 5A, FIG. 13A). The treatment with the stable androgen analog, R1881, had no effect on RASAL3 mRNA expression level in NAF; however, it significantly elevated RASAL3 mRNA expression in CAF. Conversely, androgen receptor antagonists, bicalutamide and enzalutamide, downregulated RASAL3 mRNA expression in NAF, yet had little effect on the already low basal mRNA expression of RASAL3 in the CAF. As evidence of epigenetic alteration of CAF by androgen signaling, we found that the demethylating agent, 5-aza-2′-deoxycytidine, restored RASAL3 mRNA expression in CAF, similar to that found by R1881. The expression data was supported by methylation specific PCR demonstrating the hypermethylation of the RASAL3 promoter by bicalutamide and enzalutamide and hypomethylation by R1881, similar to 5-aza-2′-deoxycytidine treatment (FIG. 13B). To quantitate methylation status of the RASAL3 exon 2 region we performed bisulfite sequencing of CAF treated with R1881. Androgen signaling was found to prevent CpG methylation of RASAL3, by 2.6-fold over the vehicle treated CAF (FIG. 5B).

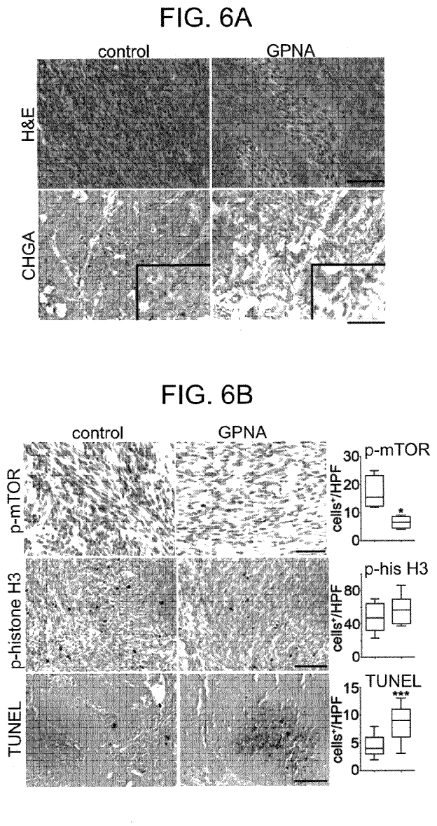

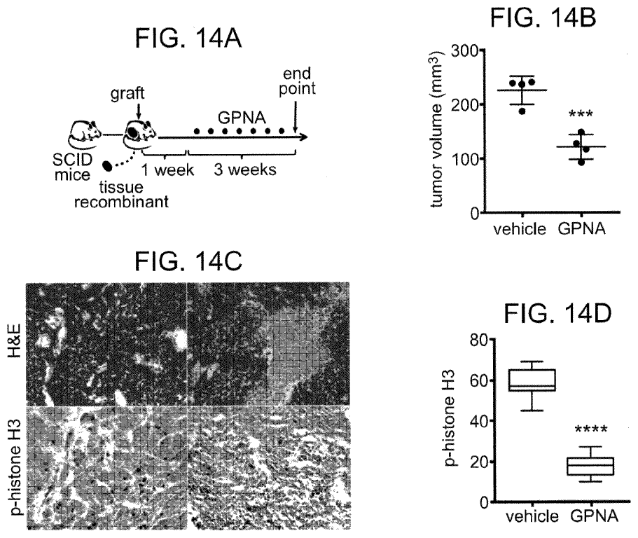

To determine the role of resulting glutamine on neuroendocrine differentiation, 3D co-cultures of CAF-CWR22Rv1 were treated with glutamine uptake or conversion blockers, GPNA or BPTES, in the context of enzalutamide. The administration of either BPTES or GPNA significantly reduced the mRNA expression of the panel of neuroendocrine biomarker genes, well below control levels (F test P<0.0001, FIG. 5C). As neuroendocrine differentiation can potentiate PCa castrate resistance, in the next set of mouse studies, we tested the role of glutamine uptake on tumor expansion. In tissue recombinant xenograft models, mice were either left intact or castrated, then treated with enzalutamide, followed by administration of vehicle or GPNA, as outlined in FIG. 5D and FIG. 14A. In intact mice, the administration of GPNA resulted in reduced tumor volume (P<0.001) and mitotic index, as determined by phosphorylated-histone H3 immunolocalization (P<0.0001, FIG. 14B-D). In the castrated, enzalutamide treated mice, we found that these tissue recombinant tumors expanded despite ADT, but the added treatment with GPNA significantly reduced tumor size (FIG. 5E, F; P<0.0001). Histological analysis revealed ADT resulted in solid sheets of cells, whereas addition of GPNA treatment was associated with karyolytic cells (FIG. 6A). In agreement with the results of the neuroendocrine gene panel, GPNA treatment reduced chromogranin A expression induced by castration and enzalutamide treatment. Accordingly, the localization of phosphorylated-mTOR (Ser-2481) was reduced (P<0.05) and cell death (TUNEL, P<0.001) was elevated by the combination treatment, compared to ADT alone (FIG. 6B). However, blocking both androgen signaling and glutamine uptake did not significantly change the mitotic index of the tumors, compared to androgen signaling blockage alone, as localized by phosphorylated histone H3 staining. These data argue that using ADT potentiates glutamine expression by prostatic fibroblasts via RASAL3 epigenetic silencing and limiting the uptake of glutamine by the epithelia can curb the ramifications of stromal Ras activity, including the support of the epithelial energy needs and neuroendocrine differentiation.

Plasma glutamine level may be prognostic for ADT sensitivity. Considering the striking finding that glutamine can induce ADT resistance, we tested if glutamine concentrations can be associated with ADT sensitivity in prostate cancer patients. We measured blood plasma glutamine concentrations in 28 PCa patients that received ADT (Table 1). Of note, the specific androgen signaling-axis interventions in the patient pool included, androgen receptor antagonists: bicalutamide, enzalutamide, and apalutamide, as well as androgen synthesis inhibitors: leuprolide, goserelin, triptorelin, and abiraterone. Of the samples analyzed, 18 patients were responsive and 10 were not responsive to ADT, based on serum prostate specific antigen (PSA) regulation (FIG. 7A). The waterfall plot illustrates patients with glutamine concentrations above 2 mM corresponded to disease progression on ADT and those with glutamine values at or below the threshold to be treatment-responsive (Fisher exact test odds ratio of 7.451, P=0.02). Compared to baseline glutamine concentrations of 0.4-0.7 mM in normal subjects, the PCa patients in this small cohort overall had markedly elevated glutamine concentrations (average=1.8 mM), in agreement with previously reported elevations in glutamate levels in high grade PCa subjects (Koochekpour et al., 2012). These results support the concept that reduced ADT-sensitivity is associated with elevated circulating glutamine concentrations.

ADT can induce epigenetic alteration of the stromal fibroblasts to promote PCa progression to a more aggressive differentiation state. Based on our earlier report that prostatic CAF have elevated DNA hypermethylation due to epithelia-derived paracrine signals (Banerjee et al., 2014), we performed a whole genome methylome analysis to reveal the silencing of a tumor suppressor, RASAL3. The outcomes of our interrogation of stromal-epithelial interactions are summarized in FIG. 7B, where the hypermethylation of the RASAL3 promoter in CAF induced RAS-dependent macropinocytosis for the lysosomal catabolism of albumin and generation of glutamine. Metabolomic profiling uncovered the role of stromal glutamine as an underlying metabolic controller facilitating cell survival and proliferation. Glutamine transporter-facilitated uptake and subsequent metabolism of stromal glutamine into epithelia for utilization by the TCA cycle and mTOR activation contribute to the energy needs of the cancer and its differentiation, respectively. This is the first demonstration of epigenetic Ras regulation in CAF consequently contributing to cancer progression. Targeting Ras signaling via RasGAPs, has been a focus of many laboratories. We demonstrated that human CAFs have low expression of RASAL3, due to exon 2 hypermethylation, resulting in Ras signaling activation. We further found that elevated Ras activity in prostatic mouse wild type fibroblasts was sufficient to promote differentiation state analogous to the tumor promoting CAF (Hayward et al., 2001; Olumi et al., 1999). A key finding of the present work adds a dimension to the previously recognized epigenetic regulation by androgen signaling. Further, the elevated glutamine levels detected in the blood of patients on androgen deprivation therapy, suggest that nearly any metastatic site would be exposed to this amino acid in support of elevated energy needs as well as neuroendocrine differentiation. Adjuvant androgen deprivation therapy is commonly administered to patients with recurrent prostate cancer. As most PCa recurrence is local, the orthotopic grafts in our studies provide a relevant basis for blocking glutamine uptake and/or metabolism. As far as distant metastasis, one could extrapolate that local tumor expansion can affect distant metastatic progression via glutamine.

To provide aspects of the present disclosure, embodiments may employ any number of programmable processing devices that execute software or stored instructions. Physical processors and/or machines employed by embodiments of the present disclosure for any processing or evaluation may include one or more networked (Internet, cloud, WAN, LAN, satellite, wired or wireless (RF, cellular, WiFi, Bluetooth, etc.)) or non-networked general purpose computer systems, microprocessors, filed programmable gate arrays (FPGAs), digital signal processors (DSPs), micro-controllers, smart devices (e.g., smart phones), computer tablets, handheld computers, and the like, programmed according to the teachings of the exemplary embodiments. In addition, the devices and subsystems of the exemplary embodiments may be implemented by the preparation of application-specific integrated circuits (ASICs) or by interconnecting an appropriate network of conventional component circuits. Thus, the exemplary embodiments are not limited to any specific combination of hardware circuitry and/or software.

Stored on any one or on a combination of computer readable media, the exemplary embodiments of the present disclosure may include software for controlling the devices and subsystems of the exemplary embodiments, for driving the devices and subsystems of the exemplary embodiments, for enabling the devices and subsystems of the exemplary embodiments to interact with a human user, and the like. Such software can include, but is not limited to, device drivers, firmware, operating systems, development tools, applications software, database management software, and the like. Computer code devices of the exemplary embodiments can include any suitable interpretable or executable code mechanism, including but not limited to scripts, interpretable programs, dynamic link libraries (DLLs), Java classes and applets, complete executable programs, and the like. Moreover, processing capabilities may be distributed across multiple processors for better performance, reliability, cost, or other benefits.

Common forms of computer-readable media may include, for example, a floppy disk, a flexible disk, a hard disk, magnetic tape, any other suitable magnetic medium, a CD-ROM, CDRW, DVD, any other suitable optical medium, punch cards, paper tape, optical mark sheets, any other suitable physical medium with patterns of holes or other optically recognizable indicia, a RAM, a PROM, an EPROM, a FLASH-EPROM, any other suitable memory chip or cartridge, a carrier wave or any other suitable medium from which a computer can read. Such storage media can also be employed to store other types of data, e.g., data organized in a database, for access, processing, and communication by the processing devices.

The various methods and techniques described above provide a number of ways to carry out the application. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, may be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Various embodiments of this application are described herein, including the best mode known to the inventors for carrying out the application. Variations on those embodiments will become apparent to those of ordinary skill in the art upon reading the foregoing description. It is contemplated that skilled artisans can employ such variations as appropriate, and the application may be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that may be employed may be within the scope of the application. Thus, by way of example, but not of limitation, alternative configurations of the embodiments of the application may be utilized in accordance with the teachings herein. Accordingly, embodiments of the present application are not limited to that precisely as shown and described.

Various embodiments of the present disclosure are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

Many variations and alternative elements have been disclosed in embodiments of the present disclosure. Still further variations and alternate elements will be apparent to one of skill in the art. Among these variations, without limitation, is the selection of steps, pharmaceutical compositions, administration routes and devices, technologies for the inventive methods, and the diseases and other clinical conditions that may be diagnosed, prognosed or treated therewith. Various embodiments of the present disclosure can specifically include or exclude any of these variations or elements.

In some embodiments, the numbers expressing quantities of ingredients, properties such as concentration, reaction conditions, and so forth, used to describe and claim certain embodiments of the present disclosure are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements.

The foregoing description of various embodiments of the present disclosure known to the applicant at this time of filing the application has been presented and is intended for the purposes of illustration and description. The present description is not intended to be exhaustive nor limit the present disclosure to the precise form disclosed and many modifications and variations are possible in the light of the above teachings. The embodiments described serve to explain the principles of the present disclosure and its practical application and to enable others skilled in the art to utilize the present disclosure in various embodiments and with various modifications as are suited to the particular use contemplated. Therefore, it is intended that the present disclosure not be limited to the particular embodiments disclosed for carrying out the present disclosure.

While particular embodiments of the present disclosure have been shown and described, it will be obvious to those skilled in the art that, based upon the teachings herein, changes and modifications may be made without departing from this present disclosure and its broader aspects and, therefore, the appended claims are to encompass within their scope all such changes and modifications as are within the true spirit and scope of this present disclosure.

1. A method of treating, reducing the severity of, slowing the progression of, or inhibiting the progression of a cancer, cancer metastasis, tumor or tumor metastasis, or sensitizing a cancer, cancer metastasis, tumor or tumor metastasis to a cancer therapy, in a subject in need thereof, comprising:
administering a glutaminase inhibitor, a glutamine metabolism inhibitor, a micropinocytosis inhibitor or a combination thereof to the subject to treat, reduce the severity of, slow the progression of, or inhibit the progression of the cancer, cancer metastasis, tumor or tumor metastasis, or to sensitize the cancer, cancer metastasis, tumor or tumor metastasis to the cancer therapy.

2. The method of claim 1,
wherein the glutaminase inhibitor is selected from the group consisting of: bis-2(5-phenylacetamido-1,3,4-thiadiazol-2-yl)ethyl sulfide (BPTES), and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839), salts thereof, and combinations thereof,
wherein the glutamine metabolism inhibitor is L-gamma-glutamyl-p-nitroanilide (GPNA) or salts thereof.

3. (canceled)

4. The method of claim 1, wherein the method is treating, reducing the severity of, slowing the progression of, or inhibiting the progression of prostate cancer, and the method further comprises
administering an androgen deprivation therapy to the subject to treat, reduce the severity of, slow the progression of, or inhibit the progression of the prostate cancer.

5. The method of claim 4, wherein androgen deprivation therapy comprises surgical castration or an antiandrogen, or wherein the androgen deprivation therapy comprises an androgen receptor signaling inhibitor, androgen receptor inhibitor, or both.

6. The method of claim 5, wherein the antiandrogen is an androgen receptor (AR) antagonist, androgen synthesis inhibitor, antigonadotropin, or combinations thereof.

7. (canceled)

8. The method of claim 4, wherein the prostate cancer is androgen deprivation therapy resistant prostate cancer.

9. The method of claim 1, wherein the method is treating, reducing the severity of, slowing the progression of, or inhibiting the progression of cancer, and the method further comprises
administering a cancer therapy to the subject to treat, reduce the severity of, slow the progression of, or inhibit the progression of the cancer.

10. The method of claim 9, wherein the method further comprises administering a CD105 inhibitor to the subject.

11. The method of claim 10, wherein the method further comprises administering an immune checkpoint inhibitor to the subject.

12. The method of claim 11, wherein the immune checkpoint inhibitor is anti-PD1 inhibitor, anti-PDL1 inhibitor, or anti-CD47 inhibitor.

13. The method of claim 12,
wherein the anti-PD1 inhibitor is selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, PF-06801591, anti-PD1 antibody expressing pluripotent killer T lymphocytes (PIK-PD-1), autologous 4SCAR-IgT cells, and combinations thereof,
wherein the anti-PDL1 inhibitor is selected from the group consisting of BGB-A333, CK-301, FAZ053, KN035, MDX-1105, MSB2311, SHR-1316, atezolizumab, avelumab, durvalumab, BMS-936559, CK-301, M7824, and combinations thereof,
wherein the anti-CD47 inhibitor is selected from the group consisting of anti-CD47 monoclonal antibody AO-176, anti-CD47 monoclonal antibody CC-90002, anti-CD47 monoclonal antibody Hu5F9-G4, anti-CD47 monoclonal antibody IBI188, anti-CD47 monoclonal antibody SHR-1603, anti-CD47 monoclonal antibody SRF231, CD47 antagonist ALX-148, chimeric humanized anti-CD47 antibody, and combinations thereof.

14. (canceled)

15. (canceled)

16. The method of claim 1, wherein the method is treating, reducing the severity of, slowing the progression of, or inhibiting the progression of cancer, and the method further comprises administering a CD105 inhibitor to the subject.

17. The method of claim 16, wherein the method further comprises administering an immune checkpoint inhibitor to the subject.

18. The method of claim 17, wherein the immune checkpoint inhibitor is anti-PD1 inhibitor, anti-PDL1 inhibitor, or anti-CD47 inhibitor.

19. The method of claim 18,
wherein the anti-PD1 inhibitor is selected from the group consisting of pembrolizumab, nivolumab, pidilizumab, AMP-224, AMP-514, spartalizumab, cemiplimab, AK105, BCD-100, BI 754091, JS001, LZM009, MGA012, Sym021, TSR-042, MGD013, AK104, XmAb20717, tislelizumab, PF-06801591, anti-PD1 antibody expressing pluripotent killer T lymphocytes (PIK-PD-1), autologous anti-EGFRvIII 4SCAR-IgT cells, and combinations thereof,
wherein the anti-PDL1 inhibitor is selected from the group consisting of BGB-A333, CK-301, FAZ053, KN035, MDX-1105, MSB2311, SHR-1316, atezolizumab, avelumab, durvalumab, BMS-936559, CK-301, M7824, and combinations thereof,
wherein the anti-CD47 inhibitor is selected from the group consisting of anti-CD47 monoclonal antibody AO-176, anti-CD47 monoclonal antibody CC-90002, anti-CD47 monoclonal antibody Hu5F9-G4, anti-CD47 monoclonal antibody IBI188, anti-CD47 monoclonal antibody SHR-1603, anti-CD47 monoclonal antibody SRF231, CD47 antagonist ALX-148, chimeric humanized anti-CD47 antibody, and combinations thereof.

20. (canceled)

21. (canceled)

22. The method of claim 1,
wherein the method is sensitizing a cancer to a cancer therapy, and the cancer is prostate cancer, and the cancer therapy is androgen deprivation therapy and the method sensitizes the prostate cancer to the androgen deprivation therapy, or
wherein the method is sensitizing a cancer to a cancer therapy, and the cancer therapy is androgen deprivation therapy, radiation therapy, chemotherapy or combinations thereof.

23. (canceled)

24. The method of claim 1, wherein the method is treating, reducing the severity of, slowing the progression of, or inhibiting the progression of cancer metastasis, and the method further comprises
administering a CD105 inhibitor; and
administering a cancer therapy.

25. The method of claim 1, wherein the cancer is selected from the group consisting of prostate cancer, pancreatic cancer, breast cancer, lung cancer, colon cancer, liver cancer, and combinations thereof.

26. The method of claim 1, wherein the cancer is a Ras mutated cancer.

27. The method of claim 1, wherein the cancer is an adenocarcinoma or a neuroendocrine cancer.

28. A method of treating, reducing the severity of, slowing the progression of, or inhibiting the progression of a disease or condition, or reducing activation of stellate cells, in a subject in need thereof, comprising:
administering a glutaminase inhibitor, a glutamine metabolism inhibitor, a micropinocytosis inhibitor or a combination thereof to the subject to treat, reduce the severity of, slow the progression of, or inhibit the progression of the condition,
wherein the condition is selected from the group consisting of liver fibrosis, pancreatic fibrosis, cancer associated depression and combinations thereof.

29. The method of claim 28,
wherein the glutaminase inhibitor is selected from the group consisting of: bis-2(5-phenylacetamido-1,3,4-thiadiazol-2-yl)ethyl sulfide (BPTES), and 2-(pyridin-2-yl)-N-(5-(4-(6-(2-(3-(trifluoromethoxy)phenyl)acetamido)pyridazin-3-yl)butyl)-1,3,4-thiadiazol-2-yl)acetamide (CB-839), salts thereof, and combinations thereof,
wherein the glutamine metabolism inhibitor is L-gamma-glutamyl-p-nitroanilide (GPNA) or salts thereof.

30. (canceled)

31. The method of claim 28, wherein the method is for reducing activation of stellate cells and the stellate cells are selected from the group consisting of hepatic stellate cells, pancreatic stellate cells, and combinations thereof.

32. A method for determining whether the amount glutamine, a glutamine metabolite or both is elevated in a subject in need thereof, comprising:
detecting the glutamine, glutamine metabolite, or both;
quantifying the amount of glutamine, glutamine metabolite, or both;
comparing the amount of the glutamine to a reference amount of glutamine, or comparing the amount of the glutamine metabolite to a reference amount of glutamine metabolite, or both; and
determining whether the amount glutamine, or a glutamine metabolite or both is elevated compared to the reference amount of glutamine, the reference amount glutamine metabolite, or both, in the subject in need thereof.

33. (canceled)

34. (canceled)

35. The method of claim 32, wherein quantifying is performed using a technique selected from the group consisting of: spectral magnetic resonance imaging, sample assay, and combinations thereof.

36. The method of claim 32, wherein the glutamine metabolite is selected from the group consisting of: glutamate, glutathione, γ-aminobutyrate, α-ketoglutarate, succinate, fumarate, malate, acetyl-CoA, 2-hydroxyglutarate and combinations thereof.

37. The method of claim 32, wherein the subject has been treated with an androgen deprivation therapy.

38. (canceled)

39. A composition, comprising:
an androgen deprivation therapy agent, and
an inhibitor selected from the group consisting of a glutaminase inhibitor, a glutamine metabolism inhibitor, a macropinocytosis inhibitor, and combinations thereof.

40. (canceled)

41. (canceled)

42. (canceled)


SEQUENCE LISTING (64 sequences)

SEQ ID NO: 1 - DNA, 20 bp, Artificial Sequence
  synthetic construct
ctcgtggcacaggattacct

SEQ ID NO: 2 - DNA, 16 bp, Artificial Sequence
  synthetic construct
agacctcgcagctgtt

SEQ ID NO: 3 - DNA, 20 bp, Artificial Sequence
  synthetic construct
tgcccagcagttttgcccat

SEQ ID NO: 4 - DNA, 20 bp, Artificial Sequence
  synthetic construct
cgttgctctccaaacctcta

SEQ ID NO: 5 - DNA, 19 bp, Artificial Sequence
  synthetic construct
cggctgcgagagaaattgc

SEQ ID NO: 6 - DNA, 22 bp, Artificial Sequence
  synthetic construct
ccactttccgttcaaggtcaag

SEQ ID NO: 7 - DNA, 28 bp, Artificial Sequence
  synthetic construct
atctatgaccttagcaatggagaatttg

SEQ ID NO: 8 - DNA, 33 bp, Artificial Sequence
  synthetic construct
gttttgatagacatatgctaatttactcccaac

SEQ ID NO: 9 - DNA, 16 bp, Artificial Sequence
  synthetic construct
ggcaagacccttggag

SEQ ID NO: 10 - DNA, 20 bp, Artificial Sequence
  synthetic construct
ccttttccccaggaagctag

SEQ ID NO: 11 - DNA, 19 bp, Artificial Sequence
  synthetic construct
cagacataaactgcgcctt

SEQ ID NO: 12 - DNA, 23 bp, Artificial Sequence
  synthetic construct
gatacacctctccaccaatgacc

SEQ ID NO: 13 - DNA, 20 bp, Artificial Sequence
  synthetic construct
accatgggtacaggctgttg

SEQ ID NO: 14 - DNA, 20 bp, Artificial Sequence
  synthetic construct
ccttctgcactgaagttgcc

SEQ ID NO: 15 - DNA, 20 bp, Artificial Sequence
  synthetic construct
cacaacaatcctcgttggac

SEQ ID NO: 16 - DNA, 20 bp, Artificial Sequence
  synthetic construct
tggtgtcacatcactccaga

SEQ ID NO: 17 - DNA, 20 bp, Artificial Sequence
  synthetic construct
cagacttgtcccgtttccat

SEQ ID NO: 18 - DNA, 20 bp, Artificial Sequence
  synthetic construct
ggtgctgactgcatcaaaga

SEQ ID NO: 19 - DNA, 20 bp, Artificial Sequence
  synthetic construct
caaggaggtgctcgattcgt

SEQ ID NO: 20 - DNA, 21 bp, Artificial Sequence
  synthetic construct
accctggttccggtgatattc

SEQ ID NO: 21 - DNA, 18 bp, Artificial Sequence
  synthetic construct
ggtctctggataagatgg

SEQ ID NO: 22 - DNA, 24 bp, Artificial Sequence
  synthetic construct
cccgttgtcagaatctccttgagg

SEQ ID NO: 23 - DNA, 20 bp, Artificial Sequence
  synthetic construct
ggcagagagacgccacacag

SEQ ID NO: 24 - DNA, 21 bp, Artificial Sequence
  synthetic construct
agtggcctttagtgcagtggt

SEQ ID NO: 25 - DNA, 23 bp, Artificial Sequence
  synthetic construct
gtgttaatggctgtgaccctgac

SEQ ID NO: 26 - DNA, 20 bp, Artificial Sequence
  synthetic construct
gagactatgacgccaactga

SEQ ID NO: 27 - DNA, 21 bp, Artificial Sequence
  synthetic construct
ggccagagttccacaaatcta

SEQ ID NO: 28 - DNA, 23 bp, Artificial Sequence
  synthetic construct
ccctccctccttcttgaaataat

SEQ ID NO: 29 - DNA, 20 bp, Artificial Sequence
  synthetic construct
ctgtgggtgctaagggtaag

SEQ ID NO: 30 - DNA, 20 bp, Artificial Sequence
  synthetic construct
gggagaagcagaaggaacaa

SEQ ID NO: 31 - DNA, 19 bp, Artificial Sequence
  synthetic construct
aaaccgtcccagcgttaat

SEQ ID NO: 32 - DNA, 21 bp, Artificial Sequence
  synthetic construct
cggctgacaatatcgctactc

SEQ ID NO: 33 - DNA, 20 bp, Artificial Sequence
  synthetic construct
agtggaccacatcaactcca

SEQ ID NO: 34 - DNA, 21 bp, Artificial Sequence
  synthetic construct
tgtccagtttctcttgctcca

SEQ ID NO: 35 - DNA, 20 bp, Artificial Sequence
  synthetic construct
ttccatgccagtggataaca

SEQ ID NO: 36 - DNA, 20 bp, Artificial Sequence
  synthetic construct
gagaggacctcaatgatgca

SEQ ID NO: 37 - DNA, 19 bp, Artificial Sequence
  synthetic construct
ctccctgtgaacagcccta

SEQ ID NO: 38 - DNA, 20 bp, Artificial Sequence
  synthetic construct
tgtgtcggagatgacctcaa

SEQ ID NO: 39 - DNA, 22 bp, Artificial Sequence
  synthetic construct
gtgtgccttaacctccctattc

SEQ ID NO: 40 - DNA, 21 bp, Artificial Sequence
  synthetic construct
cgaaccttgcctccagattat

SEQ ID NO: 41 - DNA, 21 bp, Artificial Sequence
  synthetic construct
atcattcaccaggcaaattgc

SEQ ID NO: 42 - DNA, 21 bp, Artificial Sequence
  synthetic construct
ggcttcagcataaagcgtttg

SEQ ID NO: 43 - DNA, 20 bp, Artificial Sequence
  synthetic construct
agtacctccggagaggacac

SEQ ID NO: 44 - DNA, 17 bp, Artificial Sequence
  synthetic construct
ggaacgccgcttctcca

SEQ ID NO: 45 - DNA, 24 bp, Artificial Sequence
  synthetic construct
ccaggcagtaaggtagaaatagag

SEQ ID NO: 46 - DNA, 21 bp, Artificial Sequence
  synthetic construct
gcagggacctgaagttcatta

SEQ ID NO: 47 - DNA, 25 bp, Artificial Sequence
  synthetic construct
tcacccacactgtgcccatctacga

SEQ ID NO: 48 - DNA, 25 bp, Artificial Sequence
  synthetic construct
cagcggaaccgctcattgccaatgg

SEQ ID NO: 49 - DNA, 23 bp, Artificial Sequence
  synthetic construct
catgagaagtatgacaacagcct

SEQ ID NO: 50 - DNA, 22 bp, Artificial Sequence
  synthetic construct
agtccttccacgataccaaagt

SEQ ID NO: 51 - DNA, 21 bp, Artificial Sequence
  synthetic construct
cagcgacacccactccacctt

SEQ ID NO: 52 - DNA, 21 bp, Artificial Sequence
  synthetic construct
catgaggtccaccctgttgct

SEQ ID NO: 53 - DNA, 26 bp, Artificial Sequence
  synthetic construct
tgagggtagtatgtggatagatattg

SEQ ID NO: 54 - DNA, 25 bp, Artificial Sequence
  synthetic construct
actcctaataactcaaaaccctacc

SEQ ID NO: 55 - DNA, 22 bp, Artificial Sequence
  synthetic construct
gggtagggttttgagttaggag

SEQ ID NO: 56 - DNA, 25 bp, Artificial Sequence
  synthetic construct
ataaattcttatacctcccccaaaa

SEQ ID NO: 57 - DNA, 25 bp, Artificial Sequence
  synthetic construct
ttttgggggaggtataagaatttat

SEQ ID NO: 58 - DNA, 23 bp, Artificial Sequence
  synthetic construct
aaaacaaacaaaacccaaacatc

SEQ ID NO: 59 - DNA, 20 bp, Artificial Sequence
  synthetic construct
ctcgtggcacaggattacct

SEQ ID NO: 60 - DNA, 16 bp, Artificial Sequence
  synthetic construct
agacctcgcagctgtt

SEQ ID NO: 61 - DNA, 22 bp, Artificial Sequence
  synthetic construct
ttatcgttggtatatagggggc

SEQ ID NO: 62 - DNA, 24 bp, Artificial Sequence
  synthetic construct
aaaccttaaaaaatcgaaaacgac

SEQ ID NO: 63 - DNA, 24 bp, Artificial Sequence
  synthetic construct
tttattgttggtatatagggggtg

SEQ ID NO: 64 - DNA, 25 bp, Artificial Sequence
  synthetic construct
aaaaccttaaaaaatcaaaaacaac